(19) United States
(12) Patent Application Publication
(10) Pub. No.: US 2025/0320304 A1
(43) Pub. Date: Oct. 16, 2025

(54) COMPOSITIONS COMPRISING ENHANCED MULTISPECIFIC BINDING AGENTS FOR AN IMMUNE RESPONSE

(71) Applicant: Janssen Biotech, Inc., Horsham, PA (US)

(72) Inventors: Michael DIEM, Havertown, PA (US); Chichi HUANG, Malvern, PA (US); Jinquan LUO, Malvern, PA (US); Alexey TEPLYAKOV, Middletown, MD (US); Lauren BOUCHER, Waukegan, IL (US); Michael FELDKAMP, Blue Bell, PA (US); Anthony ARMSTRONG, Lawrence Township, NJ (US); Harsha Prithviraj GUNAWARDENA, Lafayette Hill, PA (US); Hirsh NANDA, Raritan, NJ (US); Michael Lawrence POLTASH, Raritan, NJ (US); Partha CHOWDHURY, Gaithersburg, MD (US); Andrew David MAHAN, Chalfont, PA (US); Elisabeth Geyer PRINSLOW, Harleysville, PA (US)

(21) Appl. No.: 18/711,312

(22) PCT Filed: Nov. 21, 2022
(86) PCT No.: PCT/IB2022/061241

§ 371 Date: May 17, 2024

Related U.S. Application Data
(60) Provisional application No. 63/281,954, filed on Nov. 22, 2021.
(60) Provisional application No. 63/322,158, filed on Mar. 21, 2022.
(60) Provisional application No. 63/393,750, filed on Jul. 29, 2022.

(51) Int. Cl. C07K 16/28

(52) U.S. Cl. CPC C07K 16/2878; C07K 16/2809; C07K 2317/24; C07K 2317/31; C07K 2317/55; C07K 2317/622; C07K 2317/732; C07K 2317/92

(57) ABSTRACT

Disclosed herein, in certain aspects, are materials and methods for molecules comprising improved single chain variable fragments.

### CROSS-REFERENCE TO RELATED APPLICATIONS

This application claims the benefit of U.S. Ser. No. 63/281,954 filed Nov. 22, 2021, U.S. Ser. No. 63/322,158 filed Mar. 21, 2022, and U.S. Ser. No. 63/393,750 filed Jul. 29, 2022, the contents of each of which are herein incorporated by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14620-710-228_SEQ_LISTING.xml”, was created on Nov. 18, 2022, and is 94,758 bytes in size.

### 1. FIELD

Disclosed herein, in various aspects, are materials and methods for making and using multispecific molecules comprising improved single chain variable fragments and equivalents thereof.

### 2. SUMMARY

In one aspect, the present disclosure provides materials and methods for molecules that are capable of binding to a target (e.g., binding molecules). In one aspect, the molecule comprises an antigen-binding fragment (Fab), a single chain variable fragment (scFv), and a fragment crystallizable region (Fc region), wherein the scFv comprises a heavy chain variable region (VH), a linker (L), and a light chain variable region (VL), wherein the scFv comprises:
a) a disulfide bond between a structurally conserved surface exposed VH position that is mutated to cysteine (Cys) and a L Cys;
b) a disulfide bond between a structurally conserved surface exposed VL position that is mutated to Cys and a L Cys; or
c) a first disulfide bond between a structurally conserved surface exposed VH Cys and a first L Cys and a second disulfide bond between a structurally conserved surface exposed VL Cys and a second L Cys. In some embodiments, the molecule has improved stability, expression yields, and/or quality as compared to a comparable molecule absent a disulfide bond or two disulfide bonds, e.g., absent the first disulfide bond and the second disulfide bond.

In some embodiments, a) the VH comprises a VH Cys at a structurally conserved surface exposed VH framework residue position and the L comprises a L Cys; b) the VL comprises a VL Cys at a structurally conserved surface exposed VL framework residue position and the L comprises a L Cys; or c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

In some embodiments, the distance between the VH Cys and the VL Cys is from about 5 Å to about 10 Å or from about 7 Å to about 9 Å.

In some embodiments, the VH Cys is at H3, H5, H40, H43, H46 or H105, wherein the residue numbering is according to Chothia.

In some embodiments, the VL Cys is at L3, L5, L39, L42, L43, L45, L100 or L102, wherein the residue numbering is according to Chothia.

In some embodiments, the VH Cys is at H105 and the VL Cys is at L42;
the VH Cys is at H43 and the VL Cys is at L100;
the VH Cys is at H3 and the VL Cys is at L3;
the VH Cys is at H3 and the VL Cys is at L5;
the VH Cys is at H3 and the VL Cys is at L39;
the VH Cys is at H3 and the VL Cys is at L42;
the VH Cys is at H3 and the VL Cys is at L45;
the VH Cys is at H3 and the VL Cys is at L100;
the VH Cys is at H3 and the VL Cys is at L102;
the VH Cys is at H5 and the VL Cys is at L3;
the VH Cys is at H5 and the VL Cys is at L5;
the VH Cys is at H5 and the VL Cys is at L39;
the VH Cys is at H5 and the VL Cys is at L42;
the VH Cys is at H5 and the VL Cys is at L45;
the VH Cys is at H5 and the VL Cys is at L100;
the VH Cys is at H5 and the VL Cys is at L102;
the VH Cys is at H40 and the VL Cys is at L3;
the VH Cys is at H40 and the VL Cys is at L5;
the VH Cys is at H40 and the VL Cys is at L39;
the VH Cys is at H40 and the VL Cys is at L42;
the VH Cys is at H40 and the VL Cys is at L45;
the VH Cys is at H40 and the VL Cys is at L100;
the VH Cys is at H40 and the VL Cys is at L102;
the VH Cys is at H43 and the VL Cys is at L3;
the VH Cys is at H43 and the VL Cys is at L5;
the VH Cys is at H43 and the VL Cys is at L39;
the VH Cys is at H43 and the VL Cys is at L42;
the VH Cys is at H43 and the VL Cys is at L45;
the VH Cys is at H43 and the VL Cys is at L102;
the VH Cys is at H46 and the VL Cys is at L3;
the VH Cys is at H46 and the VL Cys is at L5;
the VH Cys is at H46 and the VL Cys is at L39;
the VH Cys is at H46 and the VL Cys is at L42;
the VH Cys is at H46 and the VL Cys is at L45;
the VH Cys is at H46 and the VL Cys is at 100;
the VH Cys is at H46 and the VL Cys is at L102;
the VH Cys is at H105 and the VL Cys is at L3;
the VH Cys is at H105 and the VL Cys is at L5;
the VH Cys is at H105 and the VL Cys is at L39;
the VH Cys is at H105 and the VL Cys is at L45;
the VH Cys is at H105 and the VL Cys is at L100;
the VH Cys is at H105 and the VL Cys is at L102, or
the VH Cys is at H105 and the VL Cys is at L43,
wherein the residue numbering is according to Chothia.

In some embodiments, the L comprises a contiguous amino acid sequence derived from an immunoglobulin (Ig) hinge region. In some embodiments, the Ig hinge region is derived from a human Ig hinge region or a non-human Ig hinge region. In some embodiments, the Ig hinge region is derived from a human Ig hinge region.

In some embodiments, the human Ig hinge region is an IgG1, IgG2, IgG3, or IgG4 isotype.

In some embodiments, the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

In some embodiments, the L comprises an amino acid sequence C(X)_{y}C (SEQ ID NO: 24), wherein X is Gly, Ser or Pro, and y is an integer from 1 to 3.

In some embodiments, the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51), or CPSGC (SEQ ID NO: 52).

In some embodiments, the L comprises from about 14 to about 19 amino acids. In some embodiments, the L comprises about 14, about 15, about 16, about 17, about 18, or about 19 amino acids. In some embodiments, the L has a length of from about 14 to about 19 amino acids. In some embodiments, the L has a length of about 14, about 15, about 16, about 17, about 18, or about 19 amino acids.

In some embodiments, the L comprises the amino acid sequence (X)ₘC(X)_{y}C(X)ₙ (SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 27); wherein X is Gly or Pro, m is an integer from 6 to 9, y is an integer from 1 to 3 and n is an integer from 4 to 6.

In some embodiments, the L comprises the amino acid sequence of SEQ ID NO: 2, SEQ ID NO: 3, SEQ ID NO: 4, SEQ ID NO: 5, SEQ ID NO: 6, or SEQ ID NO: 7.

In some embodiments, the scFv is in the VL-L-VH orientation. In some embodiments, the scFv is in the VH-L-VL orientation.

In some embodiments, the VH comprises a Cys at H105; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H105; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H105; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation

In some embodiments, the VH comprises a Cys at H43; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the L comprises the amino acid sequence of SEQ ID NO: 3. In some embodiments, the L comprises the amino acid sequence of SEQ ID NO: 6. In some embodiments, the L comprises the amino acid sequence of SEQ ID NO: 7.

In some embodiments, the binding molecules comprises a heavy chain, a light chain, and a polypeptide, wherein the N-terminus of the heavy chain and the light chain form the Fab; wherein the polypeptide comprises the scFv at the N-terminus; and wherein the C-terminus of the polypeptide and the C-terminus of the heavy chain form the Fc region.

In some embodiments, the Fab binds to a tumor antigen and the scFv binds to a T cell antigen. In some embodiments, the tumor antigen is BCMA and the T cell antigen is CD3.

In some embodiments, the scFv comprises the amino acid sequence of SEQ ID NO: 126 or SEQ ID NO: 128.

In some embodiments, the Fab comprises (i) a VH comprising the amino acid sequence of SEQ ID NO: 132, and a VL comprising the amino acid sequence of SEQ ID NO: 129; or (ii) a VH comprising the amino acid sequence of SEQ ID NO: 137, and a VL comprising the amino acid sequence of SEQ ID NO: 135.

In some embodiments, the VH comprises a Cys at H105; the VL comprises a Cys at L43; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one aspect, the present disclosure provides a molecule comprising an antigen-binding fragment (Fab) that binds to a first antigen, and a single chain variable fragment (scFv) that binds to a second antigen, and a fragment crystallizable region (Fc region), wherein the scFv comprises a means for stabilizing the scFv.

In some embodiments, the scFv comprises a heavy chain variable region (VH), a linker (L), and a light chain variable region (VL), and wherein the means for stabilizing the scFv comprises: a) a disulfide bond between a structurally conserved surface exposed VH cysteine (Cys) and a L Cys; b) a disulfide bond between a structurally conserved surface exposed VL Cys and a L Cys; or c) a first disulfide bond between a structurally conserved surface exposed VH Cys and a first L Cys and a second disulfide bond between the structurally conserved surface exposed VL Cys and a second L Cys.

In some embodiments, a) the VH comprises a VH Cys at a structurally conserved surface exposed VH framework residue position and the L comprises a L Cys; b) the VL comprises a VL Cys at a structurally conserved surface exposed VL framework residue position and the L comprises a L Cys; or c) the VH comprises a VH Cys at a structurally conserved surface exposed VH framework residue position, the VL comprises a VL Cys at a structurally conserved surface exposed VL framework residue position, and the L comprises a first L Cys and a second L Cys, wherein the VH Cys and the first L Cys are capable of forming a disulfide bond, and the VL Cys and the second L Cys are capable of forming a disulfide bond.

In some embodiments, the distance between the VH Cys and the VL Cys is from about 5 Å to about 10 Å or from about 7 Å to about 9 Å.

In some embodiments, the VH Cys is at H3, H5, H40, H43, H46 or H105, and/or wherein the VL Cys is at L3, L5, L39, L42, L43, L45, L100 or L102, and wherein the residue numbering is according to Chothia.

In some embodiments, the VH Cys is at H105 and the VL Cys is at L42;
the VH Cys is at H43 and the VL Cys is at L100;
the VH Cys is at H3 and the VL Cys is at L3;
the VH Cys is at H3 and the VL Cys is at L5;
the VH Cys is at H3 and the VL Cys is at L39;
the VH Cys is at H3 and the VL Cys is at L42;
the VH Cys is at H3 and the VL Cys is at L45;
the VH Cys is at H3 and the VL Cys is at L100;
the VH Cys is at H3 and the VL Cys is at L102;
the VH Cys is at H5 and the VL Cys is at L3;
the VH Cys is at H5 and the VL Cys is at L5;
the VH Cys is at H5 and the VL Cys is at L39;
the VH Cys is at H5 and the VL Cys is at L42;
the VH Cys is at H5 and the VL Cys is at L45;
the VH Cys is at H5 and the VL Cys is at L100;
the VH Cys is at H5 and the VL Cys is at L102;
the VH Cys is at H40 and the VL Cys is at L3;
the VH Cys is at H40 and the VL Cys is at L5;
the VH Cys is at H40 and the VL Cys is at L39;
the VH Cys is at H40 and the VL Cys is at L42;
the VH Cys is at H40 and the VL Cys is at L45;
the VH Cys is at H40 and the VL Cys is at L100;
the VH Cys is at H40 and the VL Cys is at L102;
the VH Cys is at H43 and the VL Cys is at L3;
the VH Cys is at H43 and the VL Cys is at L5;
the VH Cys is at H43 and the VL Cys is at L39;
the VH Cys is at H43 and the VL Cys is at L42;
the VH Cys is at H43 and the VL Cys is at L45;
the VH Cys is at H43 and the VL Cys is at L102;
the VH Cys is at H46 and the VL Cys is at L3;
the VH Cys is at H46 and the VL Cys is at L5;
the VH Cys is at H46 and the VL Cys is at L39;
the VH Cys is at H46 and the VL Cys is at L42;
the VH Cys is at H46 and the VL Cys is at L45;
the VH Cys is at H46 and the VL Cys is at L100;
the VH Cys is at H46 and the VL Cys is at L102;
the VH Cys is at H105 and the VL Cys is at L3;
the VH Cys is at H105 and the VL Cys is at L5;
the VH Cys is at H105 and the VL Cys is at L39;
the VH Cys is at H105 and the VL Cys is at L45;
the VH Cys is at H105 and the VL Cys is at L100;
the VH Cys is at H105 and the VL Cys is at L102,
the VH Cys is at H105 and the VL Cys is at L43,
wherein the residue numbering is according to Chothia.

In some embodiments, the L comprises a contiguous amino acid sequence derived from an immunoglobulin (Ig) hinge region. In some embodiments, the Ig hinge region is derived from a human Ig hinge region or a non-human Ig hinge region. In some embodiments, the Ig hinge region is derived from a human Ig hinge region.

In some embodiments, the human Ig hinge region is an IgG1, IgG2, IgG3, or IgG4 isotype.

In some embodiments, the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

In some embodiments, the L comprises an amino acid sequence C(X)_{y}C (SEQ ID NO: 24), wherein X is Gly, Ser or Pro, and y is an integer from 1 to 3.

In some embodiments, the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

In some embodiments, the L comprises from about 14 to about 19 amino acids. In some embodiments, the L comprises about 14, about 15, about 16, about 17, about 18, or about 19 amino acids. In some embodiments, the L has a length of from about 14 to about 19 amino acids. In some embodiments, the L has a length of about 14, about 15, about 16, about 17, about 18, or about 19 amino acids.

In some embodiments, the L comprises the amino acid sequence (X)ₘC(X)_{y}C(X)ₙ (SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 27); wherein X is Gly or Pro, m is an integer from 6 to 9, y is an integer from 1 to 3 and n is an integer from 4 to 6.

In some embodiments, the L comprises the amino acid sequence of SEQ ID NO: 2, SEQ ID NO: 3, SEQ ID NO: 4, SEQ ID NO: 5, SEQ ID NO: 6, or SEQ ID NO: 7.

In some embodiments, the scFv is in the VL-L-VH orientation.

In some embodiments, the scFv is in the VH-L-VL orientation.

In some embodiments, the VH comprises a Cys at H105; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H105; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H105; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H5; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H3; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the VH comprises a Cys at H43; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H43; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H40; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the VH comprises a Cys at H46; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In some embodiments, the L comprises the amino acid sequence of SEQ ID NO: 3. In some embodiments, the L comprises the amino acid sequence of SEQ ID NO: 6. In some embodiments, the L comprises the amino acid sequence of SEQ ID NO: 7.

In some embodiments, the molecules comprises a heavy chain, a light chain, and a polypeptide, wherein the N-terminus of the heavy chain and the light chain form the Fab; wherein the polypeptide comprises the scFv at the N-terminus; and wherein the C-terminus of the polypeptide and the C-terminus of the heavy chain form the Fc region.

In some embodiments, the Fab binds to a tumor antigen and the scFv binds to a T cell antigen. In some embodiments, the tumor antigen is BCMA and the T cell antigen is CD3.

In some embodiments, the scFv comprises the amino acid sequence of SEQ ID NO: 126 or SEQ ID NO: 128.

In some embodiments, the Fab comprises (i) a VH comprising the amino acid sequence of SEQ ID NO: 132, and a VL comprising the amino acid sequence of SEQ ID NO: 129; or (ii) a VH comprising the amino acid sequence of SEQ ID NO: 137, and a VL comprising the amino acid sequence of SEQ ID NO: 135.

In some embodiments, the VH comprises a Cys at H105; the VL comprises a Cys at L43; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one aspect, the present disclosure provides polynucleotides encoding the molecules disclosed herein or fragments thereof, or polypeptides thereof.

In one aspect, the present disclosure provides vectors comprising the polynucleotides disclosed herein.

In one aspect, the present disclosure provides host cells comprising the vectors disclosed herein. In some embodiments, the host cell is a prokaryotic cell. In some embodiments, the host cell is an eukaryotic cell.

In one aspect, the present disclosure provides methods of producing the presently disclosed molecules. In some embodiments, the method comprises culturing the presently disclosed host cell in conditions so that the molecule is produced, and purifying the produced molecule. In some embodiments, the method comprises introducing the presently disclosed polynucleotide into a host cell; culturing the host cell in conditions so that the molecule is produced, and purifying the produced molecule.

In one aspect, the present disclosure provides compositions comprising the presently disclosed molecules. In some embodiments, the composition is a pharmaceutical composition further comprising a pharmaceutically acceptable excipient.

In one aspect, the present disclosure provides means for producing the presently disclosed molecules.

In one aspect, the present disclosure provides methods for directing or engaging a cell to a target cell. In some embodiments, the method comprises contacting the target cell with the presently disclosed molecule. In some embodiments, the Fab binds to a first antigen on the target cell and the scFv binds to a second antigen on a cell. In some embodiments, the cell is an immune cell. In some embodiments, the immune cell is a T cell. In some embodiments, the target cell is a tumor cell. In some embodiments, the method is for treating a disease or disorder in a subject. In some embodiments, the disease or disorder is a tumor. In some embodiments, the disease or disorder is cancer. In some embodiments, the subject is a human subject.

In one aspect, the present disclosure provides methods for eliminating or inhibiting a target cell. In some embodiments, the method comprises contacting the target cell with the presently disclosed molecule.

In one aspect, the present disclosure provides methods for treating a disease or disorder in a subject. In some embodiments, the method comprises administering to the subject the presently disclosed molecule.

In one aspect, the present disclosure provides the presently disclosed molecules for use as a medicament. In one aspect, the present disclosure provides molecules for use in treating a disease or disorder.

### 3. BRIEF DESCRIPTION OF THE DRAWINGS

FIG. 1 shows an exemplary design of the stabilized bispecific BCMA/CD3 antibody. The CD3 scFvs of the bispecific antibody are connected by a flexible linker and the linker is stabilized to the scFv (spFv) with disulfide bonds between the staple sequence in the linker and anchor points.

FIGS. 2A-2B show improved thermal stability of Cris7a/b domains by the stapling. FIG. 2A: Overlay of DSC thermograms for Cris7a scFv, Cris7a spFv, Cris7b scFv and Cris7b spFv. Parameters related to protein design and enthalpy features from analysis are listed in Table 12 and Table 14. FIG. 2B: SDS-PAGE of scFv and spFv proteins of Cris7a/Cris7b in LH orientation.

FIGS. 3A-3D show size exclusion chromatography of Cris7b comprising bispecific molecules.

FIGS. 4A-4D show size exclusion chromatography of CD3B219 comprising bispecific molecules.

FIG. 5 shows thermal stability of CD3/BCMA bispecific molecules.

FIG. 6 shows cytotoxicity of CD3/BCMA bispecific molecules. CD3/BCMA bispecific molecules killed BCMA⁺H929⁻GFP⁺ cells.

FIG. 7 shows activation of CD4⁺/CD25⁺ effector cells by CD3/BCMA bispecific molecules.

FIG. 8 shows activation of CD8⁺/CD25⁺ effector cells by CD3/BCMA bispecific molecules.

FIG. 9 shows yield of proteins by spFv bispecific molecules.

FIG. 10 shows aggregation resistance of spFv bispecific molecules.

FIG. 11 shows similar CD3 binding affinity of scFv bispecific molecules and spFv bispecific molecules.

FIG. 12 shows similar CD3-mediated killing properties of scFv bispecific molecules and spFv bispecific molecules.

FIGS. 13A-13E show stapling of scFvs. FIG. 13A: scFv stapling to improve low stability and minimize breathing mediated aggregation. FIG. 13B: Cartoon schematic of the “stapling” scheme, using HL configuration as an example. A similar scheme is valid for the LH construct. The dashed line indicated the flexible linker connecting the C-terminus of a leading variable region to the stapling “CPPC” motif, followed by a second dashed line connecting to the N-terminus of a trailing variable region. The segment labeled “CPPC” in the middle of the linker indicated one possible design of a “staple,” which occurs naturally in an IgG1 hinge. The anchor points (labeled “C(AP_{L})” and “C(AP_{H})”) that are mutated to Cys residues in VH and VL are shown in sticks. The short lines between the staple Cys residues to the anchor points indicate a stapling disulfide bond formation. FIG. 13C: Graphical illustration of anchor point selection geometry consideration (HL configuration) mapped onto Fv of a germline human antibody (PDB ID 5I19, GLk1). Cter: C terminus of a leading domain; Nter: N terminus of a trailing domain; APH: anchor position on a leading domain; APL: anchor position on a trailing domain; dAP: distance between the APH and the APH; d1-d4: various distances as defined in the figure. Similar illustrations can be drawn for the LH orientation (not shown). Anchor points for HL orientation are Chothia position 43 for VH (H43C) and position 100 for VL (L100C); for LH: Chothia position 42 in VL (L42C) and 105 in VH (H105C). FIG. 13D and FIG. 13E: Cβ(Cys1)-Cβ(Cys2) distance between the two Cys residues in the human IgG (PDB 5DK3) hinge CPPC (FIG. 13D), and mouse IgG2a (PDB 1IGT) hinge CPPC (FIG. 13E); These hinge Cβ(Cys1)-Cβ(Cys2) distances range from about 7 Å to about 9 Å.

FIGS. 14A-14G show structures and comparison of various scFv/spFv domains. FIG. 14A: GLk1 spFv LH. FIG. 14B: GLk1 spFv HL. FIG. 14C: GLk2 spFv HL. FIG. 14D: 2mFo-dFc electron density (contoured at 1.5σ) of the staple motif CPPC and SS to anchor points for Glk2 spFv HL. Circles indicate the stapling disulfide density. FIG. 14E: CAT2200b spFv HL. FIG. 14F: unbound CAT2200b spFv HLL as compared to CAT2200a scFv LH bound to IL-17. FIG. 14G: front and back views of unbound CAT2200b spFv HL as compared to CAT2200a spFv LH bound to IL-17.

FIG. 15 shows the staple and linker conformations in five spFv structures. The CPPC motif were re-labeled as Cys1, Pro1, Pro2, Cys2 for clarity. The structures are superimposed on the mainchain of the CPPC motif. The dashed lines indicate Cα-Cα and Cβ-Cβ distances between the Cys1 and Cys2 residues. The range of Cβ-Cβ distances observed in all copies of the linker staple Cys residues are indicated. N-termini are indicated with ‘Nter’, C-termini are indicated with ‘Cter’.

FIGS. 16A-16D show improved yields, product quality and expected disulfide formation in the stapled linker of spFv bispecific molecules. FIG. 16A: Schematic of BCMA (Fab)×CD3 (scFv/spFv) bispecific molecular architecture. HK in Fc regions indicate the knob-in-hole (K, knob; H, hole) mutations for Fc heterodimerization. RF (H435R and Y436F) mutations in the Fab-comprising chain for purification to prevent the binding of Protein A to the RF-comprising chain monomers or homodimers. FIG. 16B: SEC profiles of post-CH1 of scFv/spFv Cris7b-comprising molecules with BCMB749 indicate the presence of oligomer species (labeled 0) in the scFv proteins but absent in the spFv proteins (monomer, M). FIG. 16C: Schematic of the expected disulfides in the stapled bispecific molecules. All Cys residues are indicated by their sequential positions/numbers in their respective polypeptide chains. Expected disulfide bonds are indicated by lines connecting them. The dotted lines represent the additional disulfide bonds in the stapled region of the single chain Fv. Inter-chain disulfide bonds are shown in solid double lines. FIG. 16D: Total ion current (TIC) of the LysC-ProAlanase non-reduced digested bispecific molecules. The chromatographic peaks labelled are the fully cleaved disulfide peptides. Other peaks in the TIC represent non-specifically digested proteins of the expected disulfide peptides. Disulfide bonds with asterisks are representative species with XIC/MS1/MS2 data given in FIG. 25 and FIG. 26.

FIGS. 17A-17D show stability and retained binding affinity to CD3 of Cris7b-comprising spFv bispecific molecules. FIG. 17A: NanoDSF traces of Cris7b-comprising scFv/spFv bispecific molecules with BCMB749 showed ˜10 C transition to higher Tm with incorporation of stapling mutations. FIGS. 17B and 17C: Cris7b spFv bispecific molecules were resistant to heat induced aggregation. SEC traces (FIG. 17B) and quantification of aggregate levels (FIG. 17C) showed that Cris7b-comprising spFv bispecific molecules had a dramatic reduction in heat induced aggregation over 6 week time frame at either 4° C. or 40° C. FIG. 17D: BLI binding traces showed comparable binding features (e.g., association and dissociation) regarding their binding to recombinant CD3. Light gray: Cris7b spFv; Dark gray: Cris7b scFv Bird; Dashed lines: Cris7b G4S.

FIGS. 18A-18C show similar functions between spFv bispecific molecules and their non-stapled counterparts. FIG. 18A: spFv bispecific molecules exhibited potent killing activity of BCMA⁺ cancer cells. FIGS. 18B and 18C: scFv/spFv bispecific molecules activated CD4⁺/CD25⁺ (FIG. 18B) and CD8⁺/CD25⁺ (FIG. 18C) T cells with similar potency. The null control showed no killing or T cell activating activity.

FIGS. 19A-19B illustrate anchor points selection in VL and VH sequences. FIG. 19A: Proposed linkers between a VL (SEQ ID NO:144) and a VH (SEQ ID NO:144). The variable number of amino acid residues (aa) gives flexibility and allows proper linker-anchor disulfide formation but is not long enough to allow disulfide scrambling. FIG. 19B: The VL and VH sequences are numbered according to the Chothia numbering scheme (Chothia and Lesk 1987) and indicated the above sequences. The anchor points are highlighted in pairs with a number (1 or 2) underneath a chosen position. The two positions in VL and VH having the same highlight and number underneath represent the pair of positions used as anchor points for a specific spFv construct. Pairs 1 and 2 are for LH and HL constructs, respectively. Anchor points for HL orientation are Chothia position 43 for VH (H43C) and position 100 for VL (L100C); for LH: Chothia position 42 in VL (L42C) and 105 in VH (H105C). Glk1 VL (SEQ ID NO: 56); Glk1 VH (SEQ ID NO: 60); Glk2 VL (SEQ ID NO: 145); Glk2 VH (SEQ ID NO: 146); CAT2200 VL (SEQ ID NO: 147); CAT2200a VH (SEQ ID NO: 148).

FIGS. 20A-20E show disulfide bond geometry (FIG. 20A) and Cα-Cα and Cβ-Cβ distance distributions between anchor points for stapling (FIG. 20B and FIG. 20C) and between positions of direct interchain disulfide bonds (FIG. 20D and FIG. 20E). DS1: disulfide bond between Chothia positions L43 and H105; DS2: L100 and H44). (FIG. 20A) Cartoon to illustrate the location of Cα-Cα and Cβ-Cβ distances in a formed disulfide bond. Relative distances between Cα and Cβ residues strongly impacted the efficiency of disulfide bond formation. In evaluating the distance distributions in FIGS. 20B-20E, the two Cys residues at the anchor positions are unlikely to form disulfide bonds directly as the distances, particularly Cβ-Cβ are much longer than typical for positions that form SS bonds. For inter-VL/VH disulfide bonds (DS1 and DS2, FIG. 20D and FIG. 20E), most of the VL/VH pairs have Cβ-Cβ distances that are much wider than typical SS bond geometry, a likely structural reason that direct SS bonds did not often form or improve stability. All antibody Fab and scFv crystal structures in PDB (rcsb.org) of human, including humanized, and murine origin with a resolution of 2.5 Å and higher were included in the distance calculations. All VL/VH pairs in these structures were included. Multiple copies in the asymmetric units were treated as independent. For positions with Gly in the structure, the Gly residue was mutated to Ala without energy minimization to provide the coordinates of an estimated Cβ position. A total of 2501 Fv structures were included in the distance calculations. All calculations were carried out in MOE (CCG, Montreal) using a custom script from CCG tech support, whose assistance is acknowledged here. (FIG. 20A and FIG. 20B), Cα-Cα and Cβ-Cβ distances, respectively, for the two anchor positions selected for LH and HL stapling. (FIG. 20C and FIG. 20D) Cα-Cα and Cβ-Cβ distances, respectively, for the DS1 and DS2 in known antibody structures. The Cα and Cβ distances for protein disulfide bonds are from Dani et al. (2003) Protein Eng. 16(3):187-193.

FIGS. 21A-21C show stapling anchor to terminus geometry. FIG. 21A: Schematic illustration of the distances. Nter: N terminus; Cter: C terminus; d: distance between and VL and VH anchor points; d1-d4: d1: leading segment from domain 1; distance from C-terminus of domain 1 (VH, in cartoon) to Cys anchor residue of domain 1 (VH, in cartoon). d2: distance from C-terminus of domain 1 (VH, in cartoon) to Cys anchor residue of domain 2 (VL, in cartoon), d3: corresponding trailing segment for domain 2; distance from N-terminus of domain 2 (VL, cartoon) to Cys anchor residue of domain 2 (VL, cartoon), d4: distance from N-terminus of domain 2 (VL, cartoon) to Cys anchor residue of domain 1 (VH, cartoon). FIG. 21B and FIG. 21C: Distance distributions for the same set of Fv fragments as in FIG. S2 for the LH and HL configurations. Methods were the same as provided for FIG. 20.

FIG. 22 shows H bonding between E1 of a trailing VL domain and backbone of the trailing linker segment of Glk2 spFv HL structure.

FIG. 23 shows humanization and sequence alignment of BCMB749. Each sequence alignment comprises the parental (top), selected human acceptor germline sequence (middle) and the CDR-grafted with back mutations italicized (bottom). CDRs are underlined. Bold: CDR support positions in the framework regions. Boxed: VL/VH interface residues. BCMB749_VL (SEQ ID NO: 129); BCMB749_VH (SEQ ID NO: 132); huKV1-12*01 (SEQ ID NO: 149); huHV1-3*01 (SEQ ID NO: 150); BCMB749h_VL (SEQ ID NO:135); BCMB749h_VH (SEQ ID NO:137).

FIG. 24 shows analytical SEC traces comparing product quality of small-scale produced CD3-comprising bispecific samples with either scFv (left) or spFv (right) arms after purification. Upper plots display bispecific molecules that comprise a Cris7b variant; lower plots display bispecific molecules that comprise an alternative anti-CD3 binding variant. Plots on the left comprise a murine anti-BCMA Fab arm; plots on the right comprise a humanized anti-BCMA Fab arm.

FIGS. 25A-25C show mass spectrometry mapping of disulfides in Byos. FIG. 25A: Calculated and observed mass results for all disulfide bonded di-peptide species after LysC and ProAlanase digestions. FIG. 25B: Fc disulfide 262-322. FIG. 25C: spFv disulfide 119-237. FIG. 25B and FIG. 25C: (Upper left panel) MS1 of the expected mass is within 2 ppm of the calculated disulfide species; (Upper right panel) Extracted ion chromatogram (XIC), depicting the retention time of the expected disulfide. The signal of the recovered peptides was in the mid-range. (Bottom panel) MS/MS coverage for both peptides of the disulfide.

FIGS. 26A-26C show no impact on antibody binding by stapling. FIG. 26A: ELISA titration against recombinant CD3 showed comparable binding to an antigen, independent of the presence of scFv or spFv arm, which indicates that stapling mutations do not impede antigen binding. FIG. 26B: ELISA titration against recombinant BCMA showed comparable binding to an antigen, independent of the presence of scFv or spFv arm, which supports that stapling mutations do not impact binding of partner arm. FIG. 26C: BLI binding traces for a pair of bispecific molecules (Cris7b scFv bird linker×BCMA, left; Cris7b spFv×BCMA, right) showed comparable binding to BCMA independent of the presence of scFv or spFv arm.

FIG. 27 shows a comparison of biophysical properties of scFv/spFv bi- and tri-specifics. BsAb: bispecific antibody; TsAb: trispecific antibody, where 1 and 2 indicate target 1 and 2. sc/sp indicate format (scFv/spFv) for the single chain moiety. All affinity values by SPR. *cell binding EC50. Values for the scFv/spFv moieties only are given.

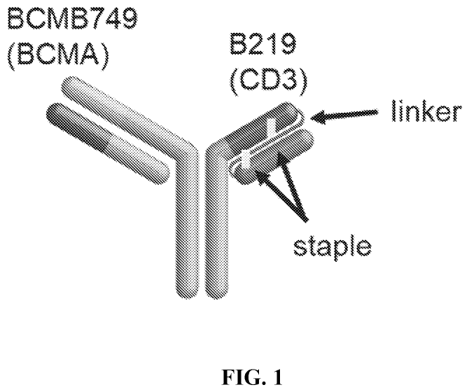

### 4. DETAILED DESCRIPTION

The disclosed methods and molecules may be understood more readily by reference to the following detailed description taken in connection with the accompanying Figures, which form a part of this disclosure. It is to be understood that the disclosed methods and molecules are not limited to the specific methods and molecules described and/or shown herein, and that the terminology used herein is for the purpose of describing particular embodiments by way of example only and is not intended to be limiting.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antibody-dependent cellular cytotoxicity,” “antibody-dependent cell-mediated cytotoxicity” or “ADCC” refers to the mechanism of inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells.

“Antibody-dependent cellular phagocytosis” or “ADCP” refers to the elimination of antibody-coated target cells by internalization by phagocytic cells, such as macrophages or dendritic cells.

The term “antigen” refers to any molecule (e.g., protein, peptide, polysaccharide, glycoprotein, glycolipid, nucleic acid, portions thereof, or combinations thereof) that is capable of mediating an immune response. Non-limiting exemplary immune responses include antibody production and activation of immune cells, such as T cells, B cells, or NK cells.

The term “antigen binding fragment” or “antigen binding domain” refers to a portion of a protein that binds an antigen. Antigen binding fragments may be synthetic, enzymatically obtainable or genetically engineered polypeptides and include portions of an immunoglobulin that bind an antigen, such as a heavy chain variable region (VH), a light chain variable region (VL), a Fab, a Fab′, F(ab′)₂, a Fd, and Fv fragments, domain antibodies (dAb) consisting of a VH domain or a VL domain, camelized VH domains, VHH domains, minimal recognition units consisting of amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific molecules comprising the antigen binding fragments. Antigen binding fragments (such as the VH and the VL) may be linked together via a linker to form various types of single antibody designs in which the VH/VL domains may pair intramolecularly, or intermolecularly in those cases when the VH and the VL domains are expressed by separate single chains, to form a monovalent antigen binding domain, such as a single chain variable fragment (scFv) or a diabody. Antigen binding fragments may also be conjugated to other antibodies, proteins, antigen binding fragments or alternative scaffolds, which may be monospecific or multispecific, to engineer bispecific and multispecific molecules.

The term “antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL are composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “bispecific” refers to a molecule (such as an antibody) that specifically binds two distinct antigens or two distinct epitopes within the same antigen. A bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The term “BCMA” refers to B cell maturation antigen. BCMA is also known as CD269, and TNFRSF17 (UniProt Q02223), and is a member of the tumor necrosis receptor superfamily that is expressed in differentiated plasma cells. In some embodiments, the BCMA is human BCMA. An exemplary human BCMA nucleotide sequence is provided by GenBank Accession Number BC058291. There are four major haplotypes of the BCMA gene in the human genome (Kawasaki et al., Genes Immun. 2:276-9, 2001). In accordance with the present disclosure, the term “BCMA” encompasses all four haplotypes. In some embodiments, the extracellular domain of human BCMA consists of amino acids 1 to 54 of the amino acid sequence having a Uniprot Ref. No. Q02223-1. The term “antibody against BCMA, anti-BCMA antibody” as used herein relates to an antibody specifically binding to BCMA. In some embodiments, the anti-BCMA antibody binds to human BCMA. In some embodiments, the anti-BCMA antibody binds to a portion of human BCMA. In some embodiments, the anti-BCMA antibody binds to the extracellular domain of human BCMA.

The term “chimeric antigen receptor” or “CAR” refers to engineered T cell receptors, which graft a ligand or antigen specificity onto immune cells, e.g., T cells (including, but not limited to, naïve T cells, central memory T cells, effector memory T cells, or combinations thereof). CARs are also known as artificial T-cell receptors, chimeric T-cell receptors or chimeric immunoreceptors. In some embodiments, a CAR comprises an extracellular domain capable of binding to an antigen, a transmembrane domain, and at least one intracellular domain. In some embodiments, the intracellular domain comprises a polypeptide known to function as a domain that transmits a signal to cause activation or inhibition of a biological process in a cell. In some embodiments, the transmembrane domain comprises a peptide or polypeptide that is known to span the cell membrane and can function to link the extracellular domain and the intracellular domain. In some embodiments, the CAR further comprises a hinge domain, which serves as a linker between the extracellular domain and the transmembrane domain.

“CD3” refers to an antigen that is expressed on T cells as part of the multimeric T cell receptor (TCR) complex. CD3 consists of a homodimer or heterodimer formed from the association of two or four receptor chains: CD3 epsilon, CD3 delta, CD3 zeta and CD3 gamma. In some embodiments, CD3 antibodies provided herein bind to a CD3-epsilon polypeptide, which, together with CD3-gamma, CD3-delta and CD3-zeta, and the T cell receptor alpha/beta and gamma/delta heterodimers, forms the T cell receptor-CD3 complex. This complex plays an important role in coupling antigen recognition to several intracellular signal-transduction pathways. The CD3 complex mediates signal transduction, resulting in T cell activation and proliferation. CD3 is required for an immune response. The term “CD3” includes any CD3 variant, isoform, and species homolog, which is naturally expressed by cells (including T cells) or can be expressed on cells transfected with genes or cDNA encoding proteins of interest. In certain embodiments, the CD3 is a human CD3.

The term “complement-dependent cytotoxicity” or “CDC”, refers to the mechanism of inducing cell death in which the Fc effector domain of a target-bound protein binds and activates complement component C1q, which in turn activates the complement cascade leading to target cell death. Activation of complement may also result in deposition of complement components on the target cell surface that facilitate CDC by binding complement receptors (e.g., CR3) on leukocytes.

The term “complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al., (1970) J Exp Med 123: 211-250); Kabat et al., Sequences of Proteins of Immunological Interest, 5th Ed. Public Health Service, National Institutes of Health, Bethesda, Md., 1991), Chothia (Chothia et al., (1987) J Mol Biol 196:901-17), IMGT (Lefranc et al., (2003) Dev Comp Immnol 27: 55-77) and AbM (Martin and Thornton (1996) J Bmol Biol 263: 800-815). The correspondence between the various delineations and variable region numbering is described (see e.g., Lefranc et al., (2003) Dev Comp Immnol 27: 55-77; Honegger and Pluckthun, J Mol Biol (2001) 309:657-670; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

The term “decrease,” “lower” or “reduce,” refers generally to the ability of a test molecule to mediate a reduced response (i.e., downstream effect) when compared to the response mediated by a control or a vehicle. Non-limiting exemplary responses include binding of a protein to its antigen or receptor, enhanced binding to FcγR, or enhanced Fc effector functions, such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more.

The term “enhance,” “promote” or “increase,” refers generally to the ability of a test molecule to mediate a greater response (i.e., downstream effect) when compared to the response mediated by a control or a vehicle. Non-limiting exemplary responses include binding of a protein to its antigen or receptor, enhanced binding to FcγR, or enhanced Fc effector functions, such as enhanced ADCC, CDC and/or ADCP. Enhance may be a statistically significant difference in the measured response between the test molecule and the control (or the vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more.

The term “expression vector” refers to a vector that can be utilized in a biological system or in a reconstituted biological system to direct the translation of a polypeptide encoded by a polynucleotide sequence present in the expression vector.

The term “heterologous” refers to a polypeptide or a polynucleotide that comprises two or more polypeptides or two or more polynucleotides, which are not found in the same relationship to each other in nature.

The term “heterologous polynucleotide” refers to a polynucleotide that comprises two or more polynucleotides, which are not found in the same relationship to each other in nature.

The term “heterologous polypeptide” refers to a polypeptide that comprises two or more polypeptides, which are not found in the same relationship to each other in nature.

The term “human antibody” refers to an antibody that is optimized to have minimal immune response when administered to a human subject. Variable regions of human antibody are derived from human immunoglobulin sequences. If a human antibody comprises a constant region or a portion thereof, the constant region is also derived from human immunoglobulin sequences. A human antibody comprises heavy and light chain variable regions that are “derived from” sequences of human origin, if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulins or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-396, and in Int. Patent Publ. No. WO2009/085462. Antibodies in which at least one CDR is derived from a non-human species are not included in the definition of “human antibody”.

The term “humanized antibody” refers to an antibody in which at least one CDR is derived from non-human species and at least one framework is derived from human immunoglobulin sequences. A humanized antibody may include substitutions in the frameworks so that the frameworks may not be exact copies of expressed human immunoglobulin or human immunoglobulin germline gene sequences.

The term “isolated” refers to a homogenous population of molecules (such as scFv or spFv of the present disclosure or heterologous proteins comprising the scFv or spFv of the present disclosure), which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to about 80%, 81%, 82%, 83%, 84%, 85%, 86%, 87%, 88%, 89%, 90%, 91%, 92%, 93%, 94%, 95%, 96%, 97%, 98%, 99%, or 100% purity.

The term “modulate” refers to either enhanced or decreased ability of a test molecule to mediate an enhanced or reduced response (i.e., downstream effect) when compared to the response mediated by a control or a vehicle.

The term “monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

The term “multispecific” refers to a molecule that binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

The term “polynucleotide” refers to a molecule comprising a chain of nucleotides covalently linked by a sugar-phosphate backbone or other equivalent covalent chemistry. cDNA is a typical example of a polynucleotide.

As used herein, the term “protein” or “polypeptide” refers to a molecule that comprises one or more polypeptides each comprised of at least two amino acid residues linked by a peptide bond. A protein may be a monomer, or a protein complex of two or more subunits, the subunits being identical or distinct. Small polypeptides of less than 50 amino acids may be referred to as “peptides”. A protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation.

The term “recombinant” refers to polynucleotides, polypeptides, vectors, viruses and other macromolecules that are prepared, expressed, created or isolated by recombinant means.

The term “single chain variable fragment”, “single chain Fv” or “scFv” refers to a single chain protein comprising a VH, a VL and a linker between the VH and the VL. The scFv may have the VL and VH in either orientation, e.g., with respect to the N- to C-terminal order of the VH and the VL. The scFv may thus be in the orientation VL-linker-VH or VH-linker-VL. scFv may be engineered to comprise disulfide bonds between the VH, the VL and the linker.

The term “specifically binds,” “specific binding,” “specifically binding” or “binds” refers to a protein (such as a scFv) binding to an antigen or an epitope within the antigen with a greater binding affinity than for other antigens. Typically, the protein (such as the scFv) binds to the antigen or the epitope within the antigen with an equilibrium dissociation constant (K_{D}) of about 1×10⁻⁶ M or less, about 1×10⁻⁷ M or less, about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein).

The term “staple” refers to a scFv linker that comprises one or two Cys residues that are capable of forming a disulfide bond with the anchor point Cys.

The term “stapled single chain Fv” or “spFv” refers to a scFv that comprises one or more disulfide bonds between the VH and the linker or between the VL and the linker. In some embodiments, the spFv comprises one disulfide bond between the VH and the linker, one disulfide bond between the VL and the linker, or two disulfide bonds with one between the VH and one between the linker and the VL and the linker. In some embodiments, scFv molecules that comprise disulfide bonds between the VH and the VL are excluded from the term “spFv”.

The term “subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein. In some embodiments, the subject is a human subject.

The term “therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual.

The term “treat,” “treating” or “treatment” of a disease or disorder refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

The term “trispecific” refers to a molecule (such as an antibody) that specifically binds three distinct antigens or three distinct epitopes within the same antigen. The trispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between three or more distinct antigens.

The term “variant,” “mutant” or “altered” refers to a polypeptide or a polynucleotide that differs from a reference polypeptide or a reference polynucleotide by one or more modifications, for example one or more substitutions, insertions, and/or deletions.

The numbering of amino acid residues of an antibody constant region throughout the present disclosure is according to the EU index as described in Kabat et al., Sequences of Proteins of Immunological Interest, 5th Ed. Public Health Service, National Institutes of Health, Bethesda, MD. (1991), unless otherwise explicitly stated.

Mutations in the Ig constant regions are referred to as follows: L351Y_F405A_Y407V refers to L351Y, F405A and Y407V mutations in one immunoglobulin constant region. L351Y_F405A_Y407V/T394W refers to L351Y, F405A and Y407V mutations in a first Ig constant region and T394W mutation in a second Ig constant region present in the molecule.

The numbering of the variable regions is according to Chothia unless otherwise explicitly stated.

The term “VH Cysteine” or “VH Cys” refers to a Cys residue that resides in a VH framework.

The term “VL Cysteine” or “VL Cys” refers to a Cys residue that resides in a VL framework.

The term “stabilized” refers to a scFv retaining comparable binding to an antigen when compared to a non-heated scFv sample, which is referred to as thermostable.

The term “improved stability” refers to a spFv of the present disclosure having an elevated melting point (Tm) when compared to a parent scFv that is devoid of disulfide bonds and Cys residues introduced into the spFv. The elevated Tm may be an elevation of about 2° C. or more, such as about 3° C., 4° C., 5° C., 6° C., 7° C., 8° C., 9° C., 10° C., 11° C., 12° C., 13° C., 14° C. or 15° C.

The term “anchor point” refers to a scFv VH or a VL framework Cys residue that can be mutagenized to Cys without an adverse effect to the overall scFv structure and is capable of forming a disulfide bond with a Cys residing in a scFv linker.

The term “surface exposed” refers to an amino acid residue that is at least partially exposed to a surface of a protein and accessible to solvent, such as accessible to deuteriation. Algorithms are well-known in the art for predicting surface accessibility of residues based on a primary sequence or a protein. Alternatively, surface exposed residues may be identified from a crystal structure of a protein.

The term “LTBR” refers to a polypeptide that is a cell surface receptor for lymphotoxin involved in apoptosis and cytokine release, which is a member of the tumor necrosis factor receptor superfamily. LTBR can also be referred to as “tumor necrosis factor receptor superfamily member 3 (TNFRSF3).” LTBR is expressed on the surface of many cell types, including cells of epithelial and myeloid lineages. LTBR can bind the lymphotoxin membrane form (a complex of lymphotoxin-alpha and lymphotoxin-beta). Activation of LTBR can trigger apoptosis via TRAF3 and TRAF5 and can lead to release of interleukin 8. In some embodiments, the LTBR is a human LTBR. An exemplary human LTBR comprises the amino acid sequence with a UniProt number P36941.

### 4.1. Compositions of Matter

Antigen binding single chain variable fragments (scFv) are molecules that can be utilized as therapeutics, imaging agents, diagnostic agents, or as portions of heterologous molecules such as multispecific molecules, and the like in view of the art and the extensive teachings in the present specification. Challenges of scFvs include their low stability and tendencies to aggregate (see e.g., Worn and Pluckthun (2001) J Mol Biol 305: 989-1010; Rothlisberger et al., (2005) J Mol Biol 347: 773-789; Gross et al., (1989) Transplant Proc 21(1 Pt 1): 127-130, Porter et al., (2011) J Cancer 2: 331-332; Porter et al., (2011) N Engl J Med 365: 725-733).

Against the background, the inventors recognized the need for improved materials and methods for scFv designs that may be optionally incorporated into various molecules, including, but not limited to multispecific molecules and heterologous molecules. The present disclosure provides stabilized scFv molecules, herein referred to as spFv (stapled Fv), heterologous and multispecific molecules comprising the spFv, polynucleotides encoding them, vectors, host cells and methods of making and using them. The present disclosure is based, at least in part, on the identification of suitable residue positions in the VH and/or the VL (herein referred to as VH anchor point or VL anchor point) and in the flexible linker (herein referred to as staple) which may be engineered to cysteine residues resulting in formation of disulfide bonds between the linker and the variable domains in the scFv. The “stapling” strategy described herein is widely applicable to various molecules, including, but not limited to, all VH/VL domains and pre-existing scFv molecules providing, inter alia, structural identity to scFv with improved stability. The spFv described herein may be conjugated into any heterologous protein, bispecific or multispecific format, including chimeric antigen receptors (CAR), T cell redirection molecules, bispecific and multispecific molecules and may be used as therapeutic, diagnostic and detection molecules.

4.1.1. spFvs of the Present Disclosure

The present disclosure provides various spFvs. In one aspect, the present disclose provides an isolated single chain variable fragment (scFv) comprising a heavy chain variable region (VH), a linker (L), and a light chain variable region (VL), wherein the scFv comprises: a) a disulfide bond between a structurally conserved surface exposed VH cysteine (Cys) and a L Cys; b) a disulfide bond between a structurally conserved surface exposed VL Cys and a L Cys; or c) a first disulfide bond between a structurally conserved surface exposed VH Cys and a first L Cys and a second disulfide bond between a structurally conserved surface exposed VL Cys and a second L Cys.

The present disclosure also provides an isolated scFv comprising a VH, a L, and a VL, wherein a) the VH comprises a VH Cys at a structurally conserved surface exposed VH framework residue position and the L comprises a L Cys; b) the VL comprises a VL Cys at a structurally conserved surface exposed VL framework residue position and the L comprises a L Cys; or c) the VH comprises a VH Cys at a structurally conserved surface exposed VH framework residue position, the VL comprises a VL Cys at a structurally conserved surface exposed VL framework residue position, and the L comprises a first L Cys and a second L Cys, wherein the VH Cys and the first L Cys are capable of forming a disulfide bond, and the VL Cys and the second L Cys are capable of forming a disulfide bond. In some embodiments, the disulfide bond is formed during expression of the scFv.

In some embodiments, a spFv consists of one disulfide bond, which is formed between a L Cys and a VH Cys or between a L Cys and a VL Cys. In some embodiments, such spFvs are referred to as “half-anchored spFvs”. The anchor positions are the same in a spFv comprising one or two disulfide bonds. The linker Cys position may vary in the half-anchored spFvs as long as it satisfies distance and geometry requirements for disulfide bond formation with the anchor point. In some embodiments, the half-anchored spFv restrains VL/VH relative movement similar to a VL/VH pair stabilized with two disulfide bonds. Thus, a half-anchored spFv is also stabilized.

The VH and VL in the spFvs may be anchored in any orientation. For example, in some embodiments, the N-terminus of the VH is anchored to the C-terminus of the VL. In some embodiments, the C-terminus of the VH is anchored to the N-terminus of the VL Anchor positions also depend on VL and VH orientations and not all VL and VH anchor points can be paired.

In some embodiments, the presently disclosed spFvs have increased stability as compared to the parent scFvs devoid of the disulfide bond(s). Stability includes thermal stability and mechanical stability. Thermostability may be evaluated using differential thermal calorimetry (DSC), in which DSC scans are performed using heated protein samples (such as samples heated to 100° C.) followed by analyses of the resulting thermal melting profiles using 2-state or non-2-state transitions. For non-2-sate transitions, two transitions (Tm1 and Tm2) are recorded which correspond to the melting Tm of the VL and the VH domains, respectively. In some embodiments, the spFvs have increased thermal stability as compared to the parent scFv devoid of the disulfide bond(s). In some embodiments, the Tm of the spFv is about 10° C. higher than that of the parent scFv devoid of the disulfide bond(s) regardless of the Tm of the parent scFv.

In some embodiments, the presently disclosed spFvs have significantly improved yields and quality of the bispecific monomer as compared to the parent scFv devoid of the disulfide bond(s). In some embodiments, the presently disclosed spFvs have reduced aggregation upon heat stress at high concentrations as compared to the parent scFv devoid of the disulfide bond(s). In some embodiments, the presently disclosed spFv molecule is a multispecific molecule. In some embodiments, the spFv molecule is a bispecific molecule. In other embodiments, the spFv molecule is a trispecific molecule. In certain embodiments, the spFv molecule has improved developability as compared to the parent scFv devoid of the disulfide bond(s). In some embodiments, stapling can increase the success of scFv conversion, thus allowing more scFv molecules to be available as molecular building blocks for therapeutic constructs.

In some embodiments, the distance between the VH Cys and the VL Cys is from about 5 Å to about 10 Å. In some embodiments, the distance between the VH Cys and the VL Cys is from about 7 Å to about 9 Å. In some embodiments, the distance between the VH Cys and the VL Cys is about 7 Å. In some embodiments, the distance between the VH Cys and the VL Cys is about 8 Å. In some embodiments, the distance between the VH Cys and the VL Cys is about 9 Å.

In some embodiments, the VH Cys is at H3, H5, H40, H43, H46 or H105, wherein the residue numbering is according to Chothia.

In some embodiments, the VH Cys is at H3.

In some embodiments, the VH Cys is at H5.

In some embodiments, the VH Cys is at H40.

In some embodiments, the VH Cys is at H43.

In some embodiments, the VH Cys is at H46.

In some embodiments, the VH Cys is at H105

In some embodiments, the VL Cys is at L3, L5, L39, L42, L43, L45, L100 or L102, wherein the residue numbering is according to Chothia.

In some embodiments, the VL Cys is at L3.

In some embodiments, the VL Cys is at L5.

In some embodiments, the VL Cys is at L39.

In some embodiments, the VL Cys is at L42.

In some embodiments, the VL Cys is at L43.

In some embodiments, the VL Cys is at L45.

In some embodiments, the VL Cys is at L100.

In some embodiments, the VL Cys is at L102.

In some embodiments, the VH Cys is at H105 and the VL Cys is at L42.

In some embodiments, the VH Cys is at H105 and the VL Cys is at L43.

In some embodiments, the VH Cys is at H43 and the VL Cys is at L100.

In some embodiments, the VH Cys is at H3 and the VL Cys is at L3.

In some embodiments, the VH Cys is at H3 and the VL Cys is at L5.

In some embodiments, the VH Cys is at H3 and the VL Cys is at L39.

In some embodiments, the VH Cys is at H3 and the VL Cys is at L42.

In some embodiments, the VH Cys is at H3 and the VL Cys is at L45.

In some embodiments, the VH Cys is at H3 and the VL Cys is at L100.

In some embodiments, the VH Cys is at H3 and the VL Cys is at L102.

In some embodiments, the VH Cys is at H5 and the VL Cys is at L3.

In some embodiments, the VH Cys is at H5 and the VL Cys is at L5.

In some embodiments, the VH Cys is at H5 and the VL Cys is at L39.

In some embodiments, the VH Cys is at H5 and the VL Cys is at L42.

In some embodiments, the VH Cys is at H5 and the VL Cys is at L45.

In some embodiments, the VH Cys is at H5 and the VL Cys is at L100.

In some embodiments, the VH Cys is at H5 and the VL Cys is at L102.

In some embodiments, the VH Cys is at H40 and the VL Cys is at L3.

In some embodiments, the VH Cys is at H40 and the VL Cys is at L5.

In some embodiments, the VH Cys is at H40 and the VL Cys is at L39.

In some embodiments, the VH Cys is at H40 and the VL Cys is at L42.

In some embodiments, the VH Cys is at H40 and the VL Cys is at L45.

In some embodiments, the VH Cys is at H40 and the VL Cys is at L100.

In some embodiments, the VH Cys is at H40 and the VL Cys is at L102.

In some embodiments, the VH Cys is at H43 and the VL Cys is at L3.

In some embodiments, the VH Cys is at H43 and the VL Cys is at L5.

In some embodiments, the VH Cys is at H43 and the VL Cys is at L39.

In some embodiments, the VH Cys is at H43 and the VL Cys is at L42.

In some embodiments, the VH Cys is at H43 and the VL Cys is at L45.

In some embodiments, the VH Cys is at H43 and the VL Cys is at L102.

In some embodiments, the VH Cys is at H46 and the VL Cys is at L3.

In some embodiments, the VH Cys is at H46 and the VL Cys is at L5.

In some embodiments, the VH Cys is at H46 and the VL Cys is at L39.

In some embodiments, the VH Cys is at H46 and the VL Cys is at L42.

In some embodiments, the VH Cys is at H46 and the VL Cys is at L45.

In some embodiments, the VH Cys is at H46 and the VL Cys is at L100.

In some embodiments, the VH Cys is at H46 and the VL Cys is at L102.

In some embodiments, the VH Cys is at H105 and the VL Cys is at L3.

In some embodiments, the VH Cys is at H105 and the VL Cys is at L5.

In some embodiments, the VH Cys is at H105 and the VL Cys is at L39.

In some embodiments, the VH Cys is at H105 and the VL Cys is at L45.

In some embodiments, the VH Cys is at H105 and the VL Cys is at L100.

In some embodiments, the VH Cys is at H105 and the VL Cys is at L102.

The residue numbering of the VH and the VL regions is according to Chothia. Chothia numbering is well known. Other numbering systems, such as Kabat or IMGT numbering, or sequential numbering may also be used to number the VH and the VL residue positions. Table 1 shows the correspondence between Chothia, Kabat and sequential numbering for an exemplary VH, Glk1 VH (SEQ ID NO: 60). Table 2 shows the correspondence between Chothia, Kabat and sequential numbering for an exemplary VL, GLk1 VL (SEQ ID NO: 56).

TABLE 1
Correspondence between Chothia, Kabat and sequential
numbering for an exemplary VH, GLk1 VH

| | | | Amino |
|---|---|---|---|
| | | | acid |
| | | | residue |
| Chothia | Kabat | Sequential | at |
| numbering | numbering | numbering | position |
| H1 | H1 | 1 | E |
| H2 | H2 | 2 | V |
| H3 | H3 | 3 | Q |
| H4 | H4 | 4 | L |
| H5 | H5 | 5 | L |
| H6 | H6 | 6 | E |
| H7 | H7 | 7 | S |
| H8 | H8 | 8 | G |
| H9 | H9 | 9 | G |
| H10 | H10 | 10 | G |
| H11 | H11 | 11 | L |
| H12 | H12 | 12 | V |
| H13 | H13 | 13 | Q |
| H14 | H14 | 14 | P |
| H15 | H15 | 15 | G |
| H16 | H16 | 16 | G |
| H17 | H17 | 17 | S |
| H18 | H18 | 18 | L |
| H19 | H19 | 19 | R |
| H20 | H20 | 20 | L |
| H21 | H21 | 21 | S |
| H22 | H22 | 22 | C |
| H23 | H23 | 23 | A |
| H24 | H24 | 24 | A |
| H25 | H25 | 25 | S |
| H26 | H26 | 26 | G |
| H27 | H27 | 27 | F |
| H28 | H28 | 28 | T |
| H29 | H29 | 29 | F |
| H30 | H30 | 30 | S |
| H31 | H31 | 31 | S |
| H32 | H32 | 32 | Y |
| H33 | H33 | 33 | A |
| H34 | H34 | 34 | M |
| H35 | H35 | 35 | S |
| H36 | H36 | 36 | W |
| H37 | H37 | 37 | V |
| H38 | H38 | 38 | R |
| H39 | H39 | 39 | Q |
| H40 | H40 | 40 | A |
| H41 | H41 | 41 | P |
| H42 | H42 | 42 | G |
| H43 | H43 | 43 | K |
| H44 | H44 | 44 | G |
| H45 | H45 | 45 | L |
| H46 | H46 | 46 | E |
| H47 | H47 | 47 | W |
| H48 | H48 | 48 | V |
| H49 | H49 | 49 | S |
| H50 | H50 | 50 | A |
| H51 | H51 | 51 | I |
| H52 | H52 | 52 | S |
| H52A | H52A | 53 | G |
| H53 | H53 | 54 | S |
| H54 | H54 | 55 | G |
| H55 | H55 | 56 | G |
| H56 | H56 | 57 | S |
| H57 | H57 | 58 | T |
| H58 | H58 | 59 | Y |
| H59 | H59 | 60 | Y |
| H60 | H60 | 61 | A |
| H61 | H61 | 62 | D |
| H62 | H62 | 63 | S |
| H63 | H63 | 64 | V |
| H64 | H64 | 65 | K |
| H65 | H65 | 66 | G |
| H66 | H66 | 67 | R |
| H67 | H67 | 68 | F |
| H68 | H68 | 69 | T |
| H69 | H69 | 70 | I |
| H70 | H70 | 71 | S |
| H71 | H71 | 72 | R |
| H72 | H72 | 73 | D |
| H73 | H73 | 74 | N |
| H74 | H74 | 75 | S |
| H75 | H75 | 76 | K |
| H76 | H76 | 77 | N |
| H77 | H77 | 78 | T |
| H78 | H78 | 79 | L |
| H79 | H79 | 80 | Y |
| H80 | H80 | 81 | L |
| H81 | H81 | 82 | Q |
| H82 | H82 | 83 | M |
| H82A | H82A | 84 | N |
| H82B | H82B | 85 | S |
| H82C | H82C | 86 | L |
| H83 | H83 | 87 | R |
| H84 | H84 | 88 | A |
| H85 | H85 | 89 | E |
| H86 | H86 | 90 | D |
| H87 | H87 | 91 | T |
| H88 | H88 | 92 | A |
| H89 | H89 | 93 | V |
| H90 | H90 | 94 | Y |
| H91 | H91 | 95 | Y |
| H92 | H92 | 96 | C |
| H93 | H93 | 97 | A |
| H94 | H94 | 98 | K |
| H95 | H95 | 99 | Y |
| H96 | H96 | 100 | D |
| H97 | H97 | 101 | G |
| H98 | H98 | 102 | I |
| H99 | H99 | 103 | Y |
| H100 | H100 | 104 | G |
| H100A | H100A | 105 | E |
| H100B | H100B | 106 | L |
| H101 | H101 | 107 | D |
| H102 | H102 | 108 | F |
| H103 | H103 | 109 | W |
| H104 | H104 | 110 | G |
| H105 | H105 | 111 | Q |
| H106 | H106 | 112 | G |
| H107 | H107 | 113 | T |
| H108 | H108 | 114 | L |
| H109 | H109 | 115 | V |
| H110 | H110 | 116 | T |
| H111 | H111 | 117 | V |
| H112 | H112 | 118 | S |
| H113 | H113 | 119 | S |

TABLE 2
Correspondence between Chothia, Kabat and sequential
numbering for an exemplary VL, GLk1 VL

| | | | Amino |
|---|---|---|---|
| | | | acid |
| Chothia | Kabat | Sequential | residue at |
| numbering | numbering | numbering | position |
| L1 | L1 | 1 | D |
| L2 | L2 | 2 | I |
| L3 | L3 | 3 | Q |
| L4 | L4 | 4 | M |
| L5 | L5 | 5 | T |
| L6 | L6 | 6 | Q |
| L7 | L7 | 7 | S |
| L8 | L8 | 8 | P |
| L9 | L9 | 9 | S |
| L10 | LIC | 10 | S |
| L11 | L11 | 11 | L |
| L12 | L12 | 12 | S |
| L13 | L13 | 13 | A |
| L14 | L14 | 14 | S |
| L15 | L15 | 15 | V |
| L16 | L16 | 16 | G |
| L17 | L17 | 17 | D |
| L18 | L18 | 18 | R |
| L19 | L19 | 19 | V |
| L20 | L20 | 20 | T |
| L21 | L21 | 21 | I |
| L22 | L22 | 22 | T |
| L23 | L23 | 23 | C |
| L24 | L24 | 24 | R |
| L25 | L25 | 25 | A |
| L26 | L26 | 26 | S |
| L27 | L27 | 27 | Q |
| L28 | L28 | 28 | S |
| L29 | L29 | 29 | I |
| L30 | L30 | 30 | S |
| L31 | L31 | 31 | S |
| L32 | L32 | 32 | Y |
| L33 | L33 | 33 | L |
| L34 | L34 | 34 | N |
| L35 | L35 | 35 | W |
| L36 | L36 | 36 | Y |
| L37 | L37 | 37 | Q |
| L38 | L38 | 38 | Q |
| L39 | L39 | 39 | K |
| L40 | L40 | 40 | P |
| L41 | L41 | 41 | G |
| L42 | L42 | 42 | K |
| L43 | L43 | 43 | A |
| L44 | L44 | 44 | P |
| L45 | L45 | 45 | K |
| L46 | L46 | 46 | L |
| L47 | L47 | 47 | L |
| L48 | L48 | 48 | I |
| L49 | L49 | 49 | Y |
| L50 | L50 | 50 | A |
| L51 | L51 | 51 | A |
| L52 | L52 | 52 | S |
| L53 | L53 | 53 | S |
| L54 | L54 | 54 | L |
| L55 | L55 | 55 | Q |
| L56 | L56 | 56 | S |
| L57 | L57 | 57 | G |
| L58 | L58 | 58 | V |
| L59 | L59 | 59 | P |
| L60 | L60 | 60 | S |
| L61 | L61 | 61 | R |
| L62 | L62 | 62 | F |
| L63 | L63 | 63 | S |
| L64 | L64 | 64 | G |
| L65 | L65 | 65 | S |
| L66 | L66 | 66 | G |
| L67 | L67 | 67 | S |
| L68 | L68 | 68 | G |
| L69 | L69 | 69 | T |
| L70 | L70 | 70 | D |
| L71 | L71 | 71 | F |
| L72 | L72 | 72 | T |
| L73 | L73 | 73 | L |
| L74 | L74 | 74 | T |
| L75 | L75 | 75 | I |
| L76 | L76 | 76 | S |
| L77 | L77 | 77 | S |
| L78 | L78 | 78 | L |
| L79 | L79 | 79 | Q |
| L80 | L80 | 80 | P |
| L81 | L81 | 81 | E |
| L82 | L82 | 82 | D |
| L83 | L83 | 83 | F |
| L84 | L84 | 84 | A |
| L85 | L85 | 85 | T |
| L86 | L86 | 86 | Y |
| L87 | L87 | 87 | Y |
| L88 | L88 | 88 | C |
| L89 | L89 | 89 | Q |
| L90 | L90 | 90 | Q |
| L91 | L91 | 91 | S |
| L92 | L92 | 92 | Y |
| L93 | L93 | 93 | S |
| L94 | L94 | 94 | T |
| L95 | L95 | 95 | P |
| L96 | L96 | 96 | L |
| L97 | L97 | 97 | T |
| L98 | L98 | 98 | F |
| L99 | L99 | 99 | G |
| L100 | L100 | 100 | Q |
| L101 | L101 | 101 | G |
| L102 | L102 | 102 | T |
| L103 | L103 | 103 | K |
| L104 | L104 | 104 | V |
| L105 | L105 | 105 | E |
| L106 | L106 | 106 | I |
| L107 | L107 | 107 | K |
| L108 | L108 | 108 | R |

In some embodiments, the L comprises a contiguous amino acid sequence derived from an immunoglobulin (Ig) hinge region. In some embodiments, the Ig hinge region is derived from a human or a non-human Ig hinge region. Exemplary non-human Ig hinge regions are those from mouse, rat, dog, chicken and non-human primates, such as monkeys. In some embodiments, the Ig hinge region is derived from a human Ig hinge region. In some embodiments, the human Ig hinge region is an IgG1, IgG2, IgG3, IgG4, IgM, IgA or IgE isotype.

In some embodiments, the Ig hinge region includes residue 216 and terminates at residue 230 of a human IgG, wherein the residue numbering is according to the EU Index. In some instances, a lower hinge region from about residue 231 to about residue 237 may also be included in the IgG hinge region. In some embodiments, the IgG1 hinge region comprises the amino acid sequence of SEQ ID NO: 63, which is provided below. In some embodiments, the IgG1 hinge region comprises the amino acid sequence of SEQ ID NO: 64, which is provided below. The hinge regions of other Ig isotypes are well known and their amino acid sequences may be obtained for example at ImMunoGeneTics web site. In some embodiments, the Ig hinge region is an IgG2 hinge region. In some embodiments, the IgG2 hinge comprises the amino acid sequence of SEQ ID NO: 65, which is provided below.

(SEQ ID NO: 63)
EPKSCDKTHTCPPCP
(SEQ ID NO: 64)
EPKSCDKTHTCPPCPAPELLGG
(SEQ ID NO: 65)
ERKCCVECPPCP

In some embodiments, the L comprises a contiguous amino acid sequence, which is derived from an Ig hinge region. Thus, in some embodiments, the L comprises at least a portion of an Ig hinge region or at least a portion of an engineered Ig hinge region. An engineered Ig hinge region comprises one or more mutations as compared to a wild-type Ig hinge region. Non-limiting examples of mutations that may be introduced include substitutions of Cys residues (e.g., to reduce the number of Cys in the L to one or two), substitution of Pro residues, or any conservative modifications (such as conservative substitutions).

“Conservative modifications” refer to amino acid modifications that do not significantly affect or alter the binding characteristics of the antibody comprising the amino acid modifications. Conservative modifications include amino acid substitutions, additions, and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to may be made by known methods for example by PCR mutagenesis (U.S. Pat. No. 4,683,195). The resulting variant hinges may be incorporated into the spFv constructs of the disclosure and tested for their characteristics such as stability and binding to an antigen using known assays and assays described herein.

In some embodiments, the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3. Pro may be included into the L to provide rigidity. Gly may be included into the L to allow maximum flexibility. Any other amino acid may also be used in the L except for Cys and Met.

In some embodiments, the L comprises the amino acid sequence C(X)_{y}C (SEQ ID NO: 24), wherein X is Gly, Ser or Pro, and y is an integer from 1 to 3.

In some embodiments, the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

In some embodiments, the L comprises the amino acid sequence CPC.

In some embodiments, the L comprises the amino acid sequence CGC.

In some embodiments, the L comprises the amino acid sequence CSC.

In some embodiments, the L comprises the amino acid sequence CPPC (SEQ ID NO: 1).

In some embodiments, the L comprises the amino acid sequence CGPC (SEQ ID NO: 28).

In some embodiments, the L comprises the amino acid sequence CPGC (SEQ ID NO: 29).

In some embodiments, the L comprises the amino acid sequence CGGC (SEQ ID NO: 30).

In some embodiments, the L comprises the amino acid sequence CSPG (SEQ ID NO: 31).

In some embodiments, the L comprises the amino acid sequence CPSC (SEQ ID NO: 32).

In some embodiments, the L comprises the amino acid sequence CSSC (SEQ ID NO: 33).

In some embodiments, the L comprises the amino acid sequence CGSC (SEQ ID NO: 34).

In some embodiments, the L comprises the amino acid sequence CSGC (SEQ ID NO: 35).

In some embodiments, the L comprises the amino acid sequence CPPPC (SEQ ID NO: 36).

In some embodiments, the L comprises the amino acid sequence CGPPC (SEQ ID NO: 37).

In some embodiments, the L comprises the amino acid sequence CPGPC (SEQ ID NO: 38).

In some embodiments, the L comprises the amino acid sequence CPPGC (SEQ ID NO: 39).

In some embodiments, the L comprises the amino acid sequence CGGPC (SEQ ID NO: 40).

In some embodiments, the L comprises the amino acid sequence CPGGC (SEQ ID NO: 41).

In some embodiments, the L comprises the amino acid sequence CGGGC (SEQ ID NO: 42).

In some embodiments, the L comprises the amino acid sequence CSPPC (SEQ ID NO: 43).

In some embodiments, the L comprises the amino acid sequence CPSPC (SEQ ID NO: 44).

In some embodiments, the L comprises the amino acid sequence CPPSC (SEQ ID NO: 45).

In some embodiments, the L comprises the amino acid sequence CSSPC (SEQ ID NO: 46).

In some embodiments, the L comprises the amino acid sequence CPSSC (SEQ ID NO: 47).

In some embodiments, the L comprises the amino acid sequence CSSSC (SEQ ID NO: 48).

In some embodiments, the L comprises the amino acid sequence CGSPC (SEQ ID NO: 49).

In some embodiments, the L comprises the amino acid sequence CPGSC (SEQ ID NO: 50).

In some embodiments, the L comprises the amino acid sequence CSGPC (SEQ ID NO: 51).

In some embodiments, the L comprises the amino acid sequence CPSGC (SEQ ID NO: 52).

In some embodiments, the L comprises from about 15 to about 20 amino acids. In some embodiments, the L has a length of from about 15 to about 20 amino acids.

In some embodiments, the L comprises from about 14 to about 19 amino acids. In some embodiments, the L has a length of from about 14 to about 19 amino acids. In some embodiments, the L comprises about 14 amino acids. In some embodiments, the L has a length of about 14 amino acids. In some embodiments, the L comprises about 15 amino acids. In some embodiments, the L has a length of about 15 amino acids. In some embodiments, the L comprises about 16 amino acids. In some embodiments, the L has a length of about 16 amino acids. In some embodiments, the L comprises about 17 amino acids. In some embodiments, the L has a length of about 17 amino acids. In some embodiments, the L comprises about 18 amino acids. In some embodiments, the L has a length of about 18 amino acids. In some embodiments, the L comprises about 19 amino acids. In some embodiments, the L has a length of about 19 amino acids. In some embodiments, the L comprises about 20 amino acids. In some embodiments, the L has a length of about 20 amino acids.

In some embodiments, the L comprises the amino acid sequence (X)ₘC(X)_{y}C(X)ₙ (SEQ ID NO: 25), wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3, and n is an integer from 4 to 6.

In some embodiments, the L comprises the amino acid sequence (X)ₘC(X)_{y}C(X)ₙ (SEQ ID NO: 26), wherein X is Gly, Ser, Pro, Ala, Arg, Asn, Asp, Glu, Gln, His, Ile, Leu, Lys, Thr or Tyr, m is an integer from 6 to 9, y is an integer from 1 to 3, and n is an integer from 4 to 6.

In some embodiments, the L comprises the amino acid sequence (X)ₘC(X)_{y}C(X)ₙ (SEQ ID NO: 27); wherein X is Gly or Pro, m is an integer from 6 to 9, y is an integer from 1 to 3, and n is an integer from 4 to 6.

In some embodiments, the L comprises the amino acid sequence of SEQ ID NO: 2, SEQ ID NO: 3, SEQ ID NO: 4, SEQ ID NO: 5, SEQ ID NO: 6, or SEQ ID NO: 7.

In some embodiments, the L comprises the amino acid sequence of SEQ ID NO: 2. In some embodiments, the L comprises the amino acid sequence of SEQ ID NO: 3. In some embodiments, the L comprises the amino acid sequence of SEQ ID NO: 4. In some embodiments, the L comprises the amino acid sequence of SEQ ID NO: 5. In some embodiments, the L comprises the amino acid sequence of SEQ ID NO: 6. In some embodiments, the L comprises the amino acid sequence of SEQ ID NO: 7.

In some embodiments, the L has a length of from about 5 to about 10 amino acids. In some embodiments, the L comprises about 5 amino acids. In some embodiments, the L consists of about 5 amino acids. In some embodiments, the L comprises 7 amino acids. In some embodiments, the L consists of 7 amino acids. In some embodiments, the L comprises 8 amino acids. In some embodiments, the L consists of 8 amino acids. In some embodiments, the L comprises 9 amino acids. In some embodiments, the L consists of 9 amino acids. In some embodiments, the L comprises about 10 amino acids. In some embodiments, the L consists of about 10 amino acids.

In some embodiments, the L further comprises a trailing segment. In some embodiments, the trailing segment has a length of 4 amino acids. In some embodiments, the trailing segment has a length of 5 amino acids.

In some embodiments, the L comprises a 9+4+5 configuration.

In some embodiments, the spFv is in the VL-L-VH orientation. In some embodiments, the spFv is in the VH-L-VL orientation.

The present disclosure provides a scFv comprising a VH, a L and a VL, wherein the VH comprises a Cys at H105; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H105; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H105; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H5; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H5; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H5; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H3; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H3; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H3; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

The present disclosure provides a scFv comprising a VH, a L and a VL, wherein the VH comprises a Cys at H43; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H43; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H43; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure also provides a scFv comprising a VH, a L and a VL, wherein the VH comprises a Cys at H43; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure also provides a scFv comprising a VH, a L and a VL, wherein the VH comprises a Cys at H40; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure also provides a scFv comprising a VH, a L and a VL, wherein the VH comprises a Cys at H40; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure also provides a scFv comprising a VH, a L and a VL, wherein the VH comprises a Cys at H40; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H40; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H46; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H46; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H46; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H46; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

The present disclosure provides a scFv comprising a VH, a L and a VL, wherein the VH comprises a Cys at H105; the VL comprises Cys at L43; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the L comprises the amino acid sequence of SEQ ID NO: 2. In some embodiments, the L comprises the amino acid sequence of SEQ ID NO: 3. In some embodiments, the L comprises the amino acid sequence of SEQ ID NO: 4. In some embodiments, the L comprises the amino acid sequence of SEQ ID NO: 5. In some embodiments, the L comprises the amino acid sequence of SEQ ID NO: 6. In some embodiments, the L comprises the amino acid sequence of SEQ ID NO: 7.

4.1.2. Molecules Comprising the spFvs of the Present Disclosure

The present disclosure provides molecules comprising the presently disclosed spFvs (e.g., those disclosed in Section 4.1.1). In some embodiments, the molecules are multispecific molecules. In some embodiments, the molecules are heterologous molecules.

Similar to a non-stabilized scFv devoid of disulfide bond(s), the spFv of the present disclosure may be conjugated to a second molecule. Non-limiting examples of second molecules include half-life extending moieties, imaging agents, therapeutic agents, antibodies comprising various antibody formats and fragments thereof, antigen binding domains, Fc regions, and immunoglobulin heavy/light chains or fragments thereof.

In some embodiments, the molecule comprises a single chain variable fragment (scFv) comprising a heavy chain variable region (VH), a linker (L), and a light chain variable region (VL), wherein the scFv comprises a disulfide bond between a structurally conserved surface exposed VH cysteine (Cys) and a L Cys; a disulfide bond between a structurally conserved surface exposed VL Cys and a L Cys; or a first disulfide bond between a structurally conserved surface exposed VH Cys and a first L Cys and a second disulfide bond between a structurally conserved surface exposed VL Cys and a second L Cys.

In some embodiments, the molecule comprises a scFv comprising a VH, a L and a VL, wherein the VH comprises a VH Cys at a structurally conserved surface exposed VH framework residue position and the L comprises a L Cys; the VL comprises a VL Cys at a structurally conserved surface exposed VL framework residue position and the L comprises a L Cys; or the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

In some embodiments, the distance between the VH Cys and the VL Cys is from about 5 Å to about 10 Å. In some embodiments, the distance between the VH Cys and the VL Cys is from about 7 Å to about 9 Å.

In some embodiments, the VH Cys is at H3, H5, H40, H43, H46 or H105, wherein the residue numbering is according to Chothia.

In some embodiments, the VL Cys is at L3, L5, L39, L42, L43, L45, L100 or L102, wherein the residue numbering is according to Chothia.

In some embodiments, the VH Cys is at H105 and the VL Cys is at L42.

In some embodiments, the VH Cys is at H105 and the VL Cys is at L43.

In some embodiments, the VH Cys is at H43 and the VL Cys is at L100.

In some embodiments, the VH Cys is at H3 and the VL Cys is at L3.

In some embodiments, the VH Cys is at H3 and the VL Cys is at L5.

In some embodiments, the VH Cys is at H3 and the VL Cys is at L39.

In some embodiments, the VH Cys is at H3 and the VL Cys is at L42.

In some embodiments, the VH Cys is at H3 and the VL Cys is at L45.

In some embodiments, the VH Cys is at H3 and the VL Cys is at L100.

In some embodiments, the VH Cys is at H3 and the VL Cys is at L102.

In some embodiments, the VH Cys is at H5 and the VL Cys is at L3.

In some embodiments, the VH Cys is at H5 and the VL Cys is at L5.

In some embodiments, the VH Cys is at H5 and the VL Cys is at L39.

In some embodiments, the VH Cys is at H5 and the VL Cys is at L42.

In some embodiments, the VH Cys is at H5 and the VL Cys is at L45.

In some embodiments, the VH Cys is at H5 and the VL Cys is at L100.

In some embodiments, the VH Cys is at H5 and the VL Cys is at L102.

In some embodiments, the VH Cys is at H40 and the VL Cys is at L3.

In some embodiments, the VH Cys is at H40 and the VL Cys is at L5.

In some embodiments, the VH Cys is at H40 and the VL Cys is at L39.

In some embodiments, the VH Cys is at H40 and the VL Cys is at L42.

In some embodiments, the VH Cys is at H40 and the VL Cys is at L45.

In some embodiments, the VH Cys is at H40 and the VL Cys is at L100.

In some embodiments, the VH Cys is at H40 and the VL Cys is at L102.

In some embodiments, the VH Cys is at H43 and the VL Cys is at L3.

In some embodiments, the VH Cys is at H43 and the VL Cys is at L5.

In some embodiments, the VH Cys is at H43 and the VL Cys is at L39.

In some embodiments, the VH Cys is at H43 and the VL Cys is at L42.

In some embodiments, the VH Cys is at H43 and the VL Cys is at L45.

In some embodiments, the VH Cys is at H43 and the VL Cys is at L100.

In some embodiments, the VH Cys is at H43 and the VL Cys is at L102.

In some embodiments, the VH Cys is at H46 and the VL Cys is at L3.

In some embodiments, the VH Cys is at H46 and the VL Cys is at L5.

In some embodiments, the VH Cys is at H46 and the VL Cys is at L39.

In some embodiments, the VH Cys is at H46 and the VL Cys is at L42.

In some embodiments, the VH Cys is at H46 and the VL Cys is at L45.

In some embodiments, the VH Cys is at H46 and the VL Cys is at L100.

In some embodiments, the VH Cys is at H46 and the VL Cys is at L102.

In some embodiments, the VH Cys is at H105 and the VL Cys is at L3.

In some embodiments, the VH Cys is at H105 and the VL Cys is at L5.

In some embodiments, the VH Cys is at H105 and the VL Cys is at L39.

In some embodiments, the VH Cys is at H105 and the VL Cys is at L42.

In some embodiments, the VH Cys is at H105 and the VL Cys is at L45.

In some embodiments, the VH Cys is at H105 and the VL Cys is at L100.

In some embodiments, the VH Cys is at H105 and the VL Cys is at L102.

The residue numbering of the VH and the VL regions is according to Chothia.

In some embodiments, the L comprises a contiguous amino acid sequence derived from an immunoglobulin (Ig) hinge region. In some embodiments, the Ig hinge region is derived from a human Ig hinge region or a non-human Ig hinge region. Non-limiting examples of non-human Ig hinge regions include those from mouse, rat, dog, chicken and non-human primates, such as monkeys. In some embodiments, the Ig hinge region is derived from a human Ig hinge region. In some embodiments, the human Ig hinge region is an IgG1, IgG2, IgG3, IgG4, IgM, IgA or IgE isotype.

In some embodiments, the L comprises an amino acid sequence C(X)_{y}C (SEQ ID NO: 23), wherein X is Gly, Ser, Pro, Ala, Arg, Asn, Asp, Glu, Gln, His, Ile, Leu, Lys, Phe, Thr, Trp or Tyr, and y is an integer from 1 to 3. Pro may be included into the linker to provide rigidity. Gly may be included into the linker to allow maximum flexibility. Any other amino acid may also be used in the L except for Cys and Met.

In some embodiments, the L comprises the amino acid sequence C(X)_{y}C (SEQ ID NO: 24), wherein X is Gly, Ser or Pro, and y is an integer from 1 to 3.

In some embodiments, the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

In some embodiments, the L comprises from about 15 to about 20 amino acids. In some embodiments, the L has a length of from about 15 to about 20 amino acids. In some embodiments, the L comprises from about 14 to about 19 amino acids. In some embodiments, the L has a length of from about 14 to about 19 amino acids. In some embodiments, the L comprises about 14 amino acids. In some embodiments, the L has a length of about 14 amino acids. In some embodiments, the L comprises about 15 amino acids. In some embodiments, the L has a length of about 15 amino acids. In some embodiments, the L comprises about 16 amino acids. In some embodiments, the L has a length of about 16 amino acids. In some embodiments, the L comprises about 17 amino acids. In some embodiments, the L has a length of about 17 amino acids. In some embodiments, the L comprises about 18 amino acids. In some embodiments, the L has a length of about 18 amino acids. In some embodiments, the L comprises about 19 amino acids. In some embodiments, the L has a length of about 19 amino acids. In some embodiments, the L comprises about 20 amino acids. In some embodiments, the L has a length of about 20 amino acids.

In some embodiments, the L comprises the amino acid sequence (X)ₘC(X)_{y}C(X)ₙ (SEQ ID NO: 25); wherein X is Gly, Ser, Pro, Ala, Arg, Asn, Asp, Glu, Gln, His, Ile, leu, Lys, Phe Thr, Trp or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

In some embodiments, the L comprises the amino acid sequence (X)ₘC(X)_{y}C(X)ₙ (SEQ ID NO: 27); wherein X is Gly or Pro, m is an integer from 6 to 9, y is an integer from 1 to 3 and n is an integer from 4 to 6.

In some embodiments, the L comprises the amino acid sequence of SEQ ID NO: 2, SEQ ID NO: 3, SEQ ID NO: 4, SEQ ID NO: 5, SEQ ID NO: 6, or SEQ ID NO: 7.

In some embodiments, the spFv is in the VL-L-VH orientation. In some embodiments, the spFv is in the VH-L-VL orientation.

In one embodiment, provided is a molecule comprising a scFv comprising a VH, a L and a VL, wherein the VH comprises a Cys at H105; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H105; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H105; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H5; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H5; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H5; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H3; the VL comprises a Cys at L42; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H3; the VL comprises a Cys at L45; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H3; the VL comprises a Cys at L39; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In one embodiment, provided is a molecule comprising a scFv comprising a VH, a L and a VL, wherein the VH comprises a Cys at H43; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H43; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H43; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H43; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H40; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H40; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H40; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H40; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H46; the VL comprises a Cys at L100; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H46; the VL comprises a Cys at L102; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H46; the VL comprises a Cys at L5; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H46; the VL comprises a Cys at L3; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VH-L-VL orientation.

In one embodiment, provided is a molecule comprising a scFv comprising a VH, a L and a VL, wherein the VH comprises a Cys at H105; the VL comprises a Cys at L43; the L comprises the amino acid sequence of SEQ ID NO: 2, SEQ ID NO: 3, SEQ ID NO: 4, SEQ ID NO: 5, SEQ ID NO: 6, or SEQ ID NO: 7; and the scFv is in the VL-L-VH orientation.

In some embodiments, the L comprises the amino acid sequence of SEQ ID NO: 2. In some embodiments, the L comprises the amino acid sequence of SEQ ID NO: 3. In some embodiments, the L comprises the amino acid sequence of SEQ ID NO: 4. In some embodiments, the L comprises the amino acid sequence of SEQ ID NO: 5. In some embodiments, the L comprises the amino acid sequence of SEQ ID NO: 6. In some embodiments, the L comprises the amino acid sequence of SEQ ID NO: 7.

In some embodiments, the scFv of the present disclosure is conjugated to a second protein, a polynucleotide, a therapeutic agent, a cytotoxic agent, or a detectable label.

In some embodiments, the second protein is a half-life extending moiety.

In some embodiments, the second protein is an antibody or a fragment thereof.

In some embodiments, the second protein is an antigen binding fragment.

In some embodiments, the second protein is a therapeutic molecule.

4.1.2.1. Molecules Comprising the spFvs of the Present Disclosure and Half-Life Extending Moieties

The present disclosure provides, in some embodiments, molecules comprising the presently disclosed spFvs (e.g., those disclosed in Section 4.1.1) and half-life extending moieties. In some embodiments, the presently disclosed spFv is conjugated to a half-life extending moiety.

Non-limiting examples of half-life extending moieties include an immunoglobulin (Ig), a fragment of an Ig, an Ig constant region, a fragment of an Ig constant region, a Fc region, transferrin, albumin, albumin variants, an albumin binding domain, or polyethylene glycols (PEGs). Amino acid sequences of human Igs are well known. Human Igs include IgG1, IgG2, IgG3, IgG4, IgM, IgA, and IgE.

In some embodiments, the spFv of the present disclosure is conjugated to an Ig or a fragment thereof. In some embodiments, the spFv of the present disclosure is conjugated to a Fc region. In some embodiments, the spFv of the present disclosure is conjugated to transferrin. In some embodiments, the spFv of the present disclosure is conjugated to albumin. In some embodiments, the spFv of the present disclosure is conjugated to an albumin binding protein. In some embodiments, the spFv of the present disclosure is conjugated to a polyethylene glycol (PEG). Non-limiting examples of PEGs include PEG5000 and PEG20,000. In some embodiments, the spFv of the present disclosure is conjugated to a fatty acid or a fatty acid ester, e.g., for desired properties. Non-limiting examples of fatty acids and fatty acid esters include laurate, myristate, stearate, arachidate, behenate, oleate, arachidonate, octanedioic acid, tetradecanedioic acid, octadecanedioic acid, docosanedioic acid, polylysine, octane, carbohydrates (dextran, cellulose, oligo- or polysaccharides), and the like.

The half-life extending moiety may be a direct fusion with the spFv of the present disclosure and may be generated by standard cloning and expression techniques. Alternatively, well-known chemical coupling methods may be used to attach the moieties to recombinantly produced spFvs of the present disclosure.

4.1.2.2. Molecules Comprising the spFvs of the Present Disclosure and Therapeutic Agents, Cytotoxic Agents or Detectable Labels

The present disclosure provides molecules comprising the presently disclosed spFvs (e.g., those disclosed in Section 4.1.1), which are conjugated to a therapeutic agent, a cytotoxic agent, or a detectable label.

Such molecules may be used to direct therapeutics, mediate killing, visualize, identify, and/or purify cells that express the antigen to which the spFv binds to, in vitro or in vivo.

Detectable label includes compositions that, when conjugated to the presently disclosed spFv, renders the latter detectable, via, for example, spectroscopic, photochemical, biochemical, immunochemical, or chemical means.

Non-limiting examples of detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Non-limiting examples of radioactive isotopes include γ-emitting, Auger-emitting, β-emitting, an alpha-emitting, and positron-emitting radioactive isotope. Non-limiting examples of radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ^{94m}Tc, ^{99m}Tc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

In some embodiments, the metal atoms are metals with an atomic number greater than 20, including, but not limited to, calcium, scandium, titanium, vanadium, chromium, manganese, iron, cobalt, nickel, copper, zinc, gallium, germanium, arsenic, selenium, bromine, krypton, rubidium, strontium, yttrium, zirconium, niobium, molybdenum, technetium, ruthenium, rhodium, palladium, silver, cadmium, indium, tin, antimony, tellurium, iodine, xenon, cesium, barium, lanthanum, hafnium, tantalum, tungsten, rhenium, osmium, iridium, platinum, gold, mercury, thallium, lead, bismuth, francium, radium, actinium, cerium, praseodymium, neodymium, promethium, samarium, europium, gadolinium, terbium, dysprosium, holmium, erbium, thulium, ytterbium, lutetium, thorium, protactinium, uranium, neptunium, plutonium, americium, curium, berkelium, californium, einsteinium, fermium, mendelevium, nobelium, and lawrencium atoms.

In some embodiments, the metal atoms are alkaline earth metals with an atomic number greater than twenty.

In some embodiments, the metal atoms are lanthanides. In some embodiments, the metal atoms are actinides. In some embodiments, the metal atoms are transition metals. In some embodiments, the metal atoms are poor metals. In some embodiments, the metal atoms are gold atoms, bismuth atoms, tantalum atoms, and gadolinium atoms.

In some embodiments, the metal atoms are metals with an atomic number of 53 (i.e., iodine) to 83 (i.e., bismuth).

In some embodiments, the metal atoms are atoms suitable for magnetic resonance imaging.

In some embodiments, the metal atoms are metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, including, but not limited to, iron oxide, manganese oxide, or gadolinium oxide.

Suitable dyes include any commercially available dyes, including, but not limited to 5(6)-carboxyfluorescein, IRDye 680RD maleimide, IRDye 800CW, ruthenium polypyridyl dyes, and the like.

Suitable fluorophores include, but are not limited to, fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The molecule comprising the presently disclosed scFv conjugated to a detectable label may be used as an imaging agent.

In some embodiments, the detectable label is also a cytotoxic agent. In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radio-conjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including, but not limited to, tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division and have anticancer and antifungal activity. The dolastatin or auristatin drug moiety may be attached to the presently disclosed spFv through the N-terminus or the C-terminus of the peptidic drug moiety (see e.g., WO02/088172), or via any cysteine engineered into a protein.

Conjugation to a detectable label may be done using known methods.

In some embodiments, the detectable label is complexed with a chelating agent.

In some embodiments, the detectable label is conjugated to the presently disclosed spFv via a linker.

The detectable label or the cytotoxic agent may be linked directly, or indirectly, to the spFv of the present disclosure using known methods. Suitable linkers are known in the art and include, but are not limited to, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

4.1.2.3. Molecules Comprising the spFvs of the Present Disclosure and Immunoglobulin (Ig) Constant Regions or Fragments Thereof

The presently disclosed spFv may be conjugated to an Ig constant region or a fragment thereof. The present disclosure provides molecules comprising the presently disclosed spFv (e.g., one disclosed in Section 4.1.1) and an Ig constant region or a fragment thereof. In some embodiments, the Ig constant region or fragment thereof can impart antibody-like properties, including Fc effector functions C1q binding, complement dependent cytotoxicity (CDC), Fc receptor binding, antibody-dependent cell-mediated cytotoxicity (ADCC), phagocytosis or down regulation of cell surface receptors (e.g., B cell receptor; BCR). The Ig constant region or fragment thereof can also function as a half-life extending moiety as described herein. The presently disclosed spFv may also be engineered into full length antibodies using standard methods. The full length antibodies comprising the spFv may be further engineered as described herein.

An immunoglobulin heavy chain constant region is comprised of subdomains CH1, hinge, CH2 and CH3. The CH1 domain spans residues 118-215, the CH2 domain residues 231-340 and the CH3 domain residues 341-447 on the heavy chain, wherein the residue numbering is according to the EU Index. In some instances, residue 341 is referred to as a CH2 domain residue. In some embodiments, a hinge includes residue 216 and terminates at 230 of a human IgG1. In some embodiments, a hinge includes a lower hinge region from about residue 231 to about residue 237 as described herein. An Ig Fc region comprises at least the CH2 and the CH3 domains of the Ig constant region, and therefore comprises at least a region from about 231 to 447 of an Ig heavy chain constant region.

In some embodiments, the Ig constant region is a heavy chain constant region.

In some embodiments, the Ig constant region is a light chain constant region.

In some embodiments, the fragment of the Ig constant region comprises a Fc region. In some embodiments, the fragment of the Ig constant region comprises a CH2 domain. In some embodiments, the fragment of the Ig constant region comprises a CH3 domain. In some embodiments, the fragment of the Ig constant region comprises a CH2 domain and a CH3 domain. In some embodiments, the fragment of the Ig constant region comprises at least portion of a hinge, a CH2 domain and a CH3 domain. A portion of the hinge refers to one or more amino acid residues of an Ig hinge. In some embodiments, the fragment of the Ig constant region comprises a hinge, a CH2 domain and a CH3 domain.

In some embodiments, the spFv is conjugated to the N-terminus of the Ig constant region or fragment thereof. In some embodiments, the spFv is conjugated to the C-terminus of the Ig constant region or fragment thereof.

The molecule comprising the presently disclosed spFv and the Ig constant region or fragment thereof may be assessed for their functionality using several known assays. Binding to a target antigen may be assessed using methods described herein. Altered properties imparted by the Ig constant domain or fragment thereof (e.g., a Fc region) may be assayed in Fc receptor binding assays using soluble forms of the receptors, such as FcγRI, FcγRII, FcγRIII or FcRn, or using cell-based assays measuring for example ADCC, CDC or ADCP.

ADCC may be assessed using an in vitro assay using cells that express the antigen to which the spFv of the present disclosure binds to as target cells and NK cells as effector cells. Cytolysis may be detected by the release of a label (e.g., radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis is measured by measuring released BATDA into the supernatant. Data are normalized to maximal cytotoxicity with 0.67% Triton X-100 (Sigma Aldrich) and minimal control determined by spontaneous release of BATDA from target cells in the absence of any antibody.

ADCP may be evaluated by using monocyte-derived macrophages as effector cells and any cells that express the antigen to which the presently disclosed spFv binds to as target cells which are engineered to express GFP or other labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

CDC of cells may be measured for example by plating Daudi cells at 1×10⁵ cells/well (50 μL/well) in RPMI-B (RPMI supplemented with 1% BSA), adding 50 μL of a test protein to the wells at a final concentration of between 0 and 100 μg/mL, incubating the reaction for 15 min at room temperature, adding 11 μL of pooled human serum to the wells, and incubating the reaction for 45 min at 37° C. Percentage (%) lysed cells may be detected as % propidium iodide stained cells in FACS assay using standard methods.

In some embodiments, the molecule comprises an antigen-binding fragment (Fab), a single chain variable fragment (scFv), and a fragment crystallizable region (Fc region), wherein the scFv comprises a heavy chain variable region (VH), a linker (L), and a light chain variable region (VL), wherein the scFv comprises: a disulfide bond between a structurally conserved surface exposed VH cysteine (Cys) and a L Cys; a disulfide bond between a structurally conserved surface exposed VL Cys and a L Cys; or a first disulfide bond between a structurally conserved surface exposed VH Cys and a first L Cys and a second disulfide bond between a structurally conserved surface exposed VL Cys and a second L Cys, wherein the molecule has improved stability, expression yields, and/or quality as compared to a molecule absent a disulfide bond, e.g., absent the first disulfide bond and the second disulfide bond.

In some embodiments, a) the VH comprises a VH Cys at a structurally conserved surface exposed VH framework residue position and the L comprises a L Cys; b) the VL comprises a VL Cys at a structurally conserved surface exposed VL framework residue position and the L comprises a L Cys; or c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

In some embodiments, the distance between the VH Cys and the VL Cys is from about 5 Å to about 10 Å. In some embodiments, the distance between the VH Cys and the VL Cys is from about 7 Å to about 9 Å.

In some embodiments, the VH Cys is at H3, H5, H40, H43, H46 or H105, wherein the residue numbering is according to Chothia. In some embodiments, the VL Cys is at L3, L5, L39, L42, L43, L45, L100 or L102, wherein the residue numbering is according to Chothia.

In some embodiments, the VH Cys is at H105 and the VL Cys is at L42;
the VH Cys is at H43 and the VL Cys is at L100;
the VH Cys is at H3 and the VL Cys is at L3;
the VH Cys is at H3 and the VL Cys is at L5;
the VH Cys is at H3 and the VL Cys is at L39;
the VH Cys is at H3 and the VL Cys is at L42;
the VH Cys is at H3 and the VL Cys is at L45;
the VH Cys is at H3 and the VL Cys is at L100;
the VH Cys is at H3 and the VL Cys is at L102;
the VH Cys is at H5 and the VL Cys is at L3;
the VH Cys is at H5 and the VL Cys is at L5;
the VH Cys is at H5 and the VL Cys is at L39;
the VH Cys is at H5 and the VL Cys is at L42;
the VH Cys is at H5 and the VL Cys is at L45;
the VH Cys is at H5 and the VL Cys is at L100;
the VH Cys is at H5 and the VL Cys is at L102;
the VH Cys is at H40 and the VL Cys is at L3;
the VH Cys is at H40 and the VL Cys is at L5;
the VH Cys is at H40 and the VL Cys is at L39;
the VH Cys is at H40 and the VL Cys is at L42;
the VH Cys is at H40 and the VL Cys is at L45;
the VH Cys is at H40 and the VL Cys is at L100;
the VH Cys is at H40 and the VL Cys is at L102;
the VH Cys is at H43 and the VL Cys is at L3;
the VH Cys is at H43 and the VL Cys is at L5;
the VH Cys is at H43 and the VL Cys is at L39;
the VH Cys is at H43 and the VL Cys is at L42;
the VH Cys is at H43 and the VL Cys is at L45;
the VH Cys is at H43 and the VL Cys is at L102;
the VH Cys is at H46 and the VL Cys is at L3;
the VH Cys is at H46 and the VL Cys is at L5;
the VH Cys is at H46 and the VL Cys is at L39;
the VH Cys is at H46 and the VL Cys is at L42;
the VH Cys is at H46 and the VL Cys is at L45;
the VH Cys is at H46 and the VL Cys is at 100;
the VH Cys is at H46 and the VL Cys is at L102;
the VH Cys is at H105 and the VL Cys is at L3;
the VH Cys is at H105 and the VL Cys is at L5;
the VH Cys is at H105 and the VL Cys is at L39;
the VH Cys is at H105 and the VL Cys is at L45;
the VH Cys is at H105 and the VL Cys is at L100;
the VH Cys is at H105 and the VL Cys is at L102, or
the VH Cys is at H105 and the VL Cys is at L43, wherein the residue numbering is according to Chothia.

In some embodiments, the molecule comprises a scFv (or spFv) that binds CD3 and a Fab that binds BCMA. In some embodiments, the scFv comprises the amino acid sequence of SEQ ID NO: 125. In some embodiments, the scFv comprises the amino acid sequence of SEQ ID NO. 126. In some embodiments, the scFv comprises the amino acid sequence of SEQ ID NO: 127. In some embodiments, the scFv comprises the amino acid sequence of SEQ ID NO: 128. In some embodiments, the scFv comprises a VH, a L and a VL. The L links the VH and the VL. In some embodiments, the L comprises the amino acid sequence of SEQ ID NO: 2, SEQ ID NO: 3, SEQ ID NO: 4, SEQ ID NO: 5, SEQ ID NO: 6, or SEQ ID NO: 7. In some embodiments, the L comprises the amino acid sequence of SEQ ID NO: 2. In some embodiments, the linker comprises SEQ ID NO: 3. In some embodiments, the L comprises the amino acid sequence of SEQ ID NO: 4. In some embodiments, the L comprises the amino acid sequence of SEQ ID NO: 5. In some embodiments, the L comprises the amino acid sequence of SEQ ID NO: 6. In some embodiments, the L comprises the amino acid sequence of SEQ ID NO: 7. In some embodiments, the VH comprises a Cys at H105. some embodiments, the VL comprises a Cys at L43. In some embodiments, the VH comprises a Cys at H105, and the VL comprises a Cys at L43. In some embodiments, the scFv is in the VL-L-VH orientation.

In some embodiments, the scFv (or spFv) that binds to CD3 is conjugated to an Ig constant region. In some embodiments, the Ig constant region comprises the amino acid sequence of SEQ ID NO: 133. In some embodiments, the Ig constant region comprises the amino acid sequence of SEQ ID NO: 139. In some embodiments, the molecule comprises a polypeptide comprising the amino acid sequence of SEQ ID NO: 140. In some embodiments, the molecule comprises a polypeptide comprising the amino acid sequence of SEQ ID NO: 141. In some embodiments, the molecule comprises a polypeptide comprising the amino acid sequence of SEQ ID NO: 142. In some embodiments, the molecule comprises a polypeptide comprising the amino acid sequence of SEQ ID NO: 143.

In some embodiments, the Fab that binds BCMA comprises a VH and a VL. In some embodiments, the VH of the Fab comprises the amino acid sequence of SEQ ID NO: 132. In some embodiments, the VH of the Fab comprises the amino acid sequence of SEQ ID NO: 137. In some embodiments, the VL of the Fab comprises the amino acid sequence of SEQ ID NO: 129. In some embodiments, the VL of the Fab comprises the amino acid sequence of SEQ ID NO: 135. In some embodiments, the BCMA VH/VL comprises SEQ ID NO: 132 and SEQ ID NO: 129. In some embodiments, the VH of the Fab comprises the amino acid sequence of SEQ ID NO: 132 and the VL of the Fab comprises the amino acid sequence of SEQ ID NO: 135. In other embodiments, the VH of the Fab comprises SEQ ID NO: 137 and SEQ ID NO: 129. In some embodiments, the BCMA VH/VL comprises the amino acid sequence of SEQ ID NO: 137 and the VL of the Fab comprises the amino acid sequence of SEQ ID NO: 135. In a further embodiment, the BCMA VH is conjugated to an Ig constant region. In some embodiments, the Ig constant region comprises the amino acid sequence of SEQ ID NO: 133. In some embodiments, the molecule comprises a polypeptide comprising the amino acid sequence of SEQ ID NO: 134. In some embodiments, the molecule comprises a polypeptide comprising the amino acid sequence of SEQ ID NO: 138.

In some embodiments, the molecule is a bispecific molecule. In some embodiments, the bispecific molecule comprises a first polypeptide comprising the amino acid sequence of SEQ ID NO: 131 or SEQ ID NO: 136, a second polypeptide comprising the amino acid sequence of SEQ ID NO: 134 or SEQ ID NO: 138, and a third polypeptide comprising the amino acid sequence of SEQ ID NO: 140, SEQ ID NO: 141, SEQ ID NO: 142, or SEQ ID NO: 143.

In some embodiments, the bispecific molecule comprises a first polypeptide comprising the amino acid sequence of SEQ ID NO: 131, a second polypeptide comprising the amino acid sequence of SEQ ID NO: 134, and a third polypeptide comprising the amino acid sequence of SEQ ID NO: 140. In some embodiments, the bispecific molecule comprises a first polypeptide comprising the amino acid sequence of SEQ ID NO: 131, a second polypeptide comprising the amino acid sequence of SEQ ID NO: 134, and a third polypeptide comprising the amino acid sequence of SEQ ID NO: 141. In some embodiments, the bispecific molecule comprises a first polypeptide comprising the amino acid sequence of SEQ ID NO: 131, a second polypeptide comprising the amino acid sequence of SEQ ID NO: 134, and a third polypeptide comprising the amino acid sequence of SEQ ID NO: 142. In some embodiments, the bispecific molecule comprises a first polypeptide comprising the amino acid sequence of SEQ ID NO: 131, a second polypeptide comprising the amino acid sequence of SEQ ID NO: 134, and a third polypeptide comprising the amino acid sequence of SEQ ID NO: 143.

In some embodiments, the bispecific molecule comprises a first polypeptide comprising the amino acid sequence of SEQ ID NO: 131, a second polypeptide comprising the amino acid sequence of SEQ ID NO: 138, and a third polypeptide comprising the amino acid sequence of SEQ ID NO: 140. In some embodiments, the bispecific molecule comprises a first polypeptide comprising the amino acid sequence of SEQ ID NO: 131, a second polypeptide comprising the amino acid sequence of SEQ ID NO: 138, and a third polypeptide comprising the amino acid sequence of SEQ ID NO: 141. In some embodiments, the bispecific molecule comprises a first polypeptide comprising the amino acid sequence of SEQ ID NO: 131, a second polypeptide comprising the amino acid sequence of SEQ ID NO: 138, and a third polypeptide comprising the amino acid sequence of SEQ ID NO: 142. In some embodiments, the bispecific molecule comprises a first polypeptide comprising the amino acid sequence of SEQ ID NO: 131, a second polypeptide comprising the amino acid sequence of SEQ ID NO: 138, and a third polypeptide comprising the amino acid sequence of SEQ ID NO: 143.

In some embodiments, the bispecific molecule comprises a first polypeptide comprising the amino acid sequence of SEQ ID NO: 136, a second polypeptide comprising the amino acid sequence of SEQ ID NO: 134, and a third polypeptide comprising the amino acid sequence of SEQ ID NO: 140. In some embodiments, the bispecific molecule comprises a first polypeptide comprising the amino acid sequence of SEQ ID NO: 136, a second polypeptide comprising the amino acid sequence of SEQ ID NO: 134, and a third polypeptide comprising the amino acid sequence of SEQ ID NO: 141.

In some embodiments, the bispecific molecule comprises a first polypeptide comprising the amino acid sequence of SEQ ID NO: 136, a second polypeptide comprising the amino acid sequence of SEQ ID NO: 134, and a third polypeptide comprising the amino acid sequence of SEQ ID NO: 142. In some embodiments, the bispecific molecule comprises a first polypeptide comprising the amino acid sequence of SEQ ID NO: 136, a second polypeptide comprising the amino acid sequence of SEQ ID NO: 134, and a third polypeptide comprising the amino acid sequence of SEQ ID NO: 143.

In some embodiments, the bispecific molecule comprises a first polypeptide comprising the amino acid sequence of SEQ ID NO: 136, a second polypeptide comprising the amino acid sequence of SEQ ID NO: 138, and a third polypeptide comprising the amino acid sequence of SEQ ID NO: 140. In some embodiments, the bispecific molecule comprises a first polypeptide comprising the amino acid sequence of SEQ ID NO: 136, a second polypeptide comprising the amino acid sequence of SEQ ID NO: 138, and a third polypeptide comprising the amino acid sequence of SEQ ID NO: 141.

In some embodiments, the bispecific molecule comprises a first polypeptide comprising the amino acid sequence of SEQ ID NO: 136, a second polypeptide comprising the amino acid sequence of SEQ ID NO: 138, and a third polypeptide comprising the amino acid sequence of SEQ ID NO: 142. In some embodiments, the bispecific molecule comprises a first polypeptide comprising the amino acid sequence of SEQ ID NO: 136, a second polypeptide comprising the amino acid sequence of SEQ ID NO: 138, and a third polypeptide comprising the amino acid sequence of SEQ ID NO: 143.

4.1.2.4. CARs Comprising the spFvs of the Present Disclosure

The present disclosure provides chimeric antigen receptors (CARs) comprising the presently disclosed spFvs (e.g., those disclosed in Section 4.1.1). The CAR comprising the spFv of the disclosure may be monospecific or multispecific, comprising, as its extracellular domain, one or more scFvs of the present disclosure.

Chimeric antigen receptors (CARs) are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on target cells, an immune cell that expresses the CAR can target and kill the target cell.

In some embodiments, a CAR comprises an extracellular domain that binds the antigen ad an optional linker, a transmembrane domain, and an intracellular domain comprising a signaling domain.

The extracellular domain of the CAR may comprise any polypeptide that binds a desired antigen. In some embodiments, the extracellular domain of the CAR comprises the scFv (or spFv) disclosed herein. CARs may also be engineered to bind two or more desired antigens that may be arranged in tandem and separated by linker sequences. For example, one or more scFvs (or spFvs) of the present disclosure, domain antibodies, llama VHH antibodies or other VH only antibody fragments may be organized in tandem via a linker to generate bispecific or multispecific CARs.

The transmembrane domain of CAR may be derived from the transmembrane domain of CD8, an alpha, beta or zeta chain of a T-cell receptor, CD28, CD3 epsilon, CD45, CD4, CD5, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the intracellular domain of the CAR further comprises a co-stimulatory domain. The co-stimulatory domain may be derived from the intracellular domains of one or more co-stimulatory molecules. Co-stimulatory molecules are well-known cell surface molecules other than antigen receptors or Fc receptors and provide a second signal required for efficient activation and function of T lymphocytes upon binding to an antigen. Non-limiting examples of co-stimulatory molecules include 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, and ZAP70.

The intracellular domain of CAR may be derived from the signaling domains of for example CD3ζ, CD3ε, CD22, CD79a, CD66d or CD39. An intracellular domain of a CAR refers to a part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

In some embodiments, a linker is positioned between the extracellular domain and the transmembrane domain. In some embodiments, the linker is a polypeptide of about 2 to 100 amino acids in length. The linker may include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. An exemplary cleavable linker includes 2A.

An exemplary CAR comprises an extracellular domain comprising the scFv (or spFv) of the present disclosure, a transmembrane domain comprising a transmembrane domain of CD8, and an intracellular domain comprising a signaling domain of CD3ζ. An exemplary CAR comprises an extracellular domain comprising the scFv (or spFv) of the present disclosure, a transmembrane domain comprising a transmembrane domain of CD8 or a transmembrane domain of CD28, and an intracellular domain comprising a signaling domain of CD3ζ and a co-stimulatory domain comprising an intracellular domain of CD28, an intracellular domain of 4-1BB, or an intracellular domain of OX40.

CARs are generated by standard molecular biology techniques.

In some embodiments, the molecule is monospecific.

In some embodiments, the molecule is multispecific.

In some embodiments, the molecule is bispecific.

In some embodiments, the molecule is trispecific.

In some embodiments, the molecule is tetraspecific.

In some embodiments, provided herein is a scFv (e.g., spFv) structure defined by the atomic coordinates provided in Table 18. In other embodiments, provided herein is a scFv (e.g., spFv) structure defined by one or more subsets of the atomic coordinates provided in Table 18. In some embodiments, provided herein is a scFv (e.g., spFv) structure defined by the atomic coordinates provided in Table 19. In other embodiments, provided herein is a scFv (e.g., spFv) structure defined by one or more subsets of the atomic coordinates provided in Table 19. In some embodiments, provided herein is a scFv (e.g., spFv) structure defined by the atomic coordinates provided in Table 20. In other embodiments, provided herein is a scFv (e.g., spFv) structure defined by one or more subsets of the atomic coordinates provided in Table 20. In some embodiments, provided herein is a scFv (e.g., spFv) structure defined by the atomic coordinates provided in Table 21. In other embodiments, provided herein is a scFv (e.g., spFv) structure defined by one or more subsets of the atomic coordinates provided in Table 21. In some embodiments, provided herein is a scFv (e.g., spFv) structure defined by the atomic coordinates provided in Table 22. In other embodiments, provided herein is a scFv (e.g., spFv) structure defined by one or more subsets of the atomic coordinates provided in Table 22. In some embodiments, provided herein is a scFv (e.g., spFv) structure defined by the atomic coordinates provided in Table 23. In other embodiments, provided herein is a scFv (e.g., spFv) structure defined by one or more subsets of the atomic coordinates provided in Table 23.

### 4.2. Generation of Molecules Comprising the spFv of the Present Disclosure

The presently disclosed spFv may be engineered into molecules of any known format using known recombinant technologies, expression and purification protocols.

The presently disclosed spFv may be engineered into full length multispecific antibodies having one or more mutations in the CH3 domain which promoter stability of the two half molecules. These multispecific antibodies may be generated in vitro using Fab arm exchange or by co-expression of the various chains. For in vitro Fab arm exchange, two monospecific bivalent antibodies are engineered to have the one or more substitutions in the CH3 domain, the antibodies are incubated together under reducing conditions sufficient to allow the cysteines in the hinge region to undergo disulfide bond isomerization; thereby generating the multispecific antibody by Fab arm exchange. The incubation conditions may optimally be restored to non-reducing. Non-limiting examples of reducing agents that may be used include 2-mercaptoethylamine (2-MEA), dithiothreitol (DTT), dithioerythritol (DTE), glutathione, tris(2-carboxyethyl)phosphine (TCEP), L-cysteine, and beta-mercaptoethanol. In some embodiments, a reducing agent is selected from the group consisting of 2-mercaptoethylamine, dithiothreitol, and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g., Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Non-limiting examples of CH3 region mutations forming a knob and a hole include T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization include, but are not limited to, L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W, as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used include, but are not limited to, R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013/157954, WO2013/096291 and US2018/0118849.

Duobody® mutations (Genmab) are disclosed for example in US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the presently disclosed spFv may be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv)₂-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

The scFv (or spFv) of the present disclosure may also be engineered into multispecific molecules comprising three antigen binding domains. In such designs, at least one antigen binding domain is in the form of a scFv (or spFv) of the present disclosure. Exemplary designs include (in which “1” indicates the first antigen binding domain, “2” indicates the second antigen binding domain, and “3” indicates the third antigen binding domain:
Design 1: Chain A) scFv1-CH2-CH3; Chain B) VL2-CL; Chain C) VH2-CH1-hinge-CH2-CH3
Design 2: Chain A) scFv1-hinge-CH2-CH3; Chain B) VL2-CL; Chain C) VH2-CH1-hinge-CH2-CH3
Design 3: Chain A) scFv1-CH1-hinge-CH2-CH3; Chain B) VL2-CL; Chain C) VH2-CH1-hinge-CH2-CH3
Design 4: Chain A) CH2-CH3-scFv1; Chain B) VL2-CL; Chain C) VH2-CH1-hinge-CH2-CH3

CH3 engineering may be incorporated to the Designs 1-4, including, but not limited to, mutations L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

### 4.3. Isotypes, Allotypes and Fc Engineering

The Ig constant region or fragment thereof, such as a Fc region present in the presently disclosed molecules may be of any allotype or isotype.

In some embodiments, the Ig constant region or fragment thereof is an IgG1 isotype. In some embodiments, the Ig constant region or fragment thereof is an IgG2 isotype. In some embodiments, the Ig constant region or fragment thereof is an IgG3 isotype. In some embodiments, the Ig constant region or fragment thereof is an IgG4 isotype.

The Ig constant region or fragment thereof may be of any allotype. In some embodiments, the allotype has no influence on properties of the Ig constant region, such as binding or Fc-mediated effector functions. Immunogenicity of therapeutic proteins comprising Ig constant regions or fragments thereof is associated with increased risk of infusion reactions and decreased duration of therapeutic response (Baert et al., (2003) N Engl J Med 348:602-608). The extent to which therapeutic proteins comprising Ig constant regions or fragments thereof induce an immune response in the host may be determined in part by the allotype of the Ig constant region (Stickler et al., (2011) Genes and Immunity 12:213-221). Ig constant region allotype is related to amino acid sequence variations at specific locations in the constant region sequences of the antibody. Table 3 shows select IgG1, IgG2 and IgG4 allotypes.

TABLE 3
Select IgG1, IgG2 and IgG4 allotypes

| | Amino acid residue at position of | | | | | | | |
|---|---|---|---|---|---|---|---|---|
| | diversity (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| G1m(17) | | | | | K | E | M | A |
| G1m(17, 1) | | | | | K | D | L | A |

C-terminal lysine (CTL) may be removed from the Ig constant region by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA-Fe³⁺ as described in U.S. Patent Publ. No. US2014/0273092. CTL content of proteins may be measured using known methods.

In some embodiments, the spFv conjugated to the Ig constant region has a C-terminal lysine content of from about 10% to about 90%. In some embodiments, the C-terminal lysine content is from about 20% to about 80%. In some embodiments, the C-terminal lysine content is from about 40% to about 70%. In some embodiments, the C-terminal lysine content is from about 50% to about 80%. In some embodiments, the C-terminal lysine content is from about 60% to about 80%. In some embodiments, the C-terminal lysine content is from about 50% to about 70%. In some embodiments, the C-terminal lysine content is from about 60% to about 70%. In some embodiments, the C-terminal lysine content is from about 55% to about 70%. In some embodiments, the C-terminal lysine content is about 60%.

Fc region mutations may be made to the presently disclosed molecules comprising the Ig constant region or fragment thereof to modulate their effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some embodiments, the presently disclosed molecule comprises at least one mutation in the Ig constant region or fragment thereof. In some embodiments, the at least one mutation is in the Fc region.

In some embodiments, the presently disclosed molecule comprises at least one, two, three, four, five, six, seven, eight, nine, ten, eleven, twelve, thirteen, fourteen or fifteen mutations in the Fc region.

In some embodiments, the presently disclosed molecule comprises at least one mutation in the Fc region that modulates binding of the molecule to FcRn.

Fc positions that may be mutated to modulate half-life (e.g., binding to FcRn) include, but are not limited to, positions 250, 252, 253, 254, 256, 257, 307, 376, 380, 428, 434 and 435. Non-limiting examples of mutations that may be made singularly or in combination include mutations T250Q, M252Y, I253A, S254T, T256E, P257I, T307A, D376V, E380A, M428L, H433K, N434S, N434A, N434H, N434F, H435A and H435R. Non-limiting examples of singular or combination mutations that may be made to increase the half-life include mutations M428L/N434S, M252Y/S254T/T256E, T250Q/M428L, N434A and T307A/E380A/N434A. Non-limiting examples of singular or combination mutations that may be made to reduce the half-life include mutations H435A, P257I/N434H, D376V/N434H, M252Y/S254T/T256E/H433K/N434F, T308P/N434A and H435R.

In some embodiments, the presently disclosed molecule comprises M252Y/S254T/T256E mutation in the Fc region.

In some embodiments, the presently disclosed molecule comprises at least one mutation in the Fc region that reduces binding of the molecule to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of the presently disclosed molecule to the activating FcγR and subsequently to reduce effector function include, but are not limited to, positions 214, 233, 234, 235, 236, 237, 238, 265, 267, 268, 270, 295, 297, 309, 327, 328, 329, 330, 331 and 365. Non-limiting examples of mutations that may be made singularly or in combination include mutations K214T, E233P, L234V, L234A, deletion of G236, V234A, F234A, L235A, G237A, P238A, P238S, D265A, D265S, S267E, H268A, H268Q, Q268A, N297A, A327Q, P329A, D270A, Q295A, V309L, A327S, L328F, A330S and P331S in IgG1, IgG2, IgG3 or IgG4. Non-limiting examples of combination mutations that result in reduced ADCC include mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

An exemplary mutation that results in reduced CDC is a K322A mutation. A S228P mutation may be made in IgG4 to enhance IgG4 stability.

In some embodiments, the presently disclosed molecule comprises at least one mutation in the Fc region, wherein the at least one mutation is selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S. In some embodiments, the at least one mutation comprises L234A, L235A, D265S. In some embodiments, the at least one mutation comprises L234A or L235A.

In some embodiments, the presently disclosed molecule comprises at least one mutation in the Fc region that enhances binding of the molecule to FcγR and/or enhances Fc effector functions such as C1q binding, complement dependent cytotoxicity (CDC), antibody-dependent cell-mediated cytotoxicity (ADCC) and/or phagocytosis (ADCP).

Fc positions that may be mutated to increase binding of the molecule to the activating FcγR and/or enhance Fc effector functions include, but are not limited to, positions 236, 239, 243, 256, 290, 292, 298, 300, 305, 312, 326, 330, 332, 333, 334, 345, 360, 339, 378, 396 or 430 (the residue numbering is according to the EU index). Non-limiting examples of mutations that may be made singularly or in combination include G236A, S239D, F243L, T256A, K290A, R292P, S298A, Y300L, V305L, K326A, A330K, 1332E, E333A, K334A, A339T and P396L. Non-limiting examples of combination mutations that result in increased ADCC or ADCP include a S239D/I332E, S298A/E333A/K334A, F243L/R292P/Y300L, F243L/R292P/Y300L/P396L, F243L/R292P/Y300L/V305I/P396L and G236A/S239D/I332E.

Fc positions that may be mutated to enhance CDC include, but are not limited to, positions 267, 268, 324, 326, 333, 345 and 430. Non-limiting examples of mutations that may be made singularly or in combination include S267E, F1268F, S324T, K326A, K326W, E333A, E345K, E345Q, E345R, E345Y, E430S, E430F and E430T. Non-limiting examples of combination mutations that result in increased CDC include K326A/E333A, K326W/E333A, H268F/S324T, S267E/H268F, S267E/S324T and S267E/H268F/S324T.

In some embodiments, the mutations are present in a wild-type IgG1, a wild-type IgG2, or a wild-type IgG4. In some embodiments, the wild-type IgG1 comprises the amino acid sequences of SEQ ID NO: 66, which is provided below. In some embodiments, the wild-type IgG2 comprises the amino acid sequences of SEQ ID NO: 67, which is provided below. In some embodiments, the wild-type IgG4 comprises the amino acid sequences of SEQ ID NO: 68, which is provided below.

(SEQ ID NO: 66)
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
(SEQ ID NO: 67)
ASTKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQFNSTFRVVSVLT
VVHQDWLNGKEYKCKVSNKGLPAPIEKTISKTKGQPREPQVYTLPPSREEMTKNQVSLTCLV
KGFYPSDISVEWESNGQPENNYKTTPPMLDSDGSFFLYSKLTVDKSRWQQGNVFSCSVMHEA
LHNHYTQKSLSLSPGK
(SEQ ID NO: 68)
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPSCPAPEFLGGPSVFLFP
PKPKDTLMISRTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSVMHE
ALHNHYTQKSLSLSLGK

Binding of the presently disclosed molecule to FcγR or FcRn may be assessed on cells engineered to express each receptor using flow cytometry.

### 4.4. Glycoengineering

The ability of the presently disclosed molecule comprising an Ig constant region or a fragment thereof to mediate ADCC can be enhanced by engineering the oligosaccharide component of the Ig constant region or fragment thereof. Human IgG1 or IgG3 are N-glycosylated at Asn297 with the majority of the glycans in the well-known biantennary G0, GOF, G1, G1F, G2 or G2F forms. Ig constant region containing proteins may be produced by non-engineered CHO cells typically have a glycan fucose content of about at least about 85%. The removal of the core fucose from the biantennary complex-type oligosaccharides attached to the Ig constant region or fragment thereof enhances ADCC of the molecule via improved FcγRIIIa binding without altering antigen binding or CDC activity. Such molecules can be achieved using different methods reported to lead to the successful expression of relatively high defucosylated immunoglobulins bearing the biantennary complex-type of Fc oligosaccharides such as control of culture osmolality (Konno et al., (2012) Cytotechnology 64:249-265), application of a variant CHO line Lec13 as the host cell line (Shields et al., (2002) J Biol Chem 277:26733-26740), application of a variant CHO line EB66 as the host cell line (Olivier et al., (2010) MAbs; 2: 405-415), application of a rat hybridoma cell line YB2/0 as the host cell line (Shinkawa et al., (2003) J Biol Chem 278:3466-3473), introduction of small interfering RNA specifically against the a 1,6-fucosyltrasferase (FUT8) gene (Mori et al., (2004) Biotechnol Bioeng 88:901-908), or coexpression of β-1,4-N-acetylglucosaminyltransferase III and Golgi α-mannosidase II or a potent alpha-mannosidase I inhibitor, kifunensine (Ferrara et al., (2006) J Biol Chem 281:5032-5036).

In some embodiments, the presently disclosed molecule comprising the Ig constant region or fragment thereof has a biantennary glycan structure with fucose content of about between about 1% to about 15%, for example, about 15%, 14%, 13%, 12%, 11% 10%, 9%, 8%, 7%, 6%, 5%, 4%, 3%, 2%, or 1%. In some embodiments, the presently disclosed molecule comprising the Ig constant region or fragment thereof has a glycan structure with fucose content of about 50%, 40%, 45%, 40%, 35%, 30%, 25%, or 20%.

“Fucose content” refers to the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g., complex, hybrid and oligo- and high-mannose structures) as described in Int Pat. Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS); 5) Separation of the mAb oligosaccharides from the mAb protein by specific enzymatic deglycosylation with PNGase F at Asn 297. The oligosaccharides thus released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” refers to the presently disclosed molecule comprising the Ig constant region or fragment thereof with fucose content of about between about 1% and about 15%.

“Normal fucose” or “normal fucose content” refers to the presently disclosed molecule comprising the Ig constant region or fragment thereof with fucose content of great than about 50%, e.g., greater than about 80% or greater than about 85%.

### 4.5. Anti-Idiotypic Antibodies

Anti-idiotypic antibodies are antibodies that specifically bind to the presently disclosed spFv. The present disclose also provides anti-idiotypic antibodies that specifically binds to the presently disclosed spFv.

In some embodiments, the anti-idiotypic antibody binds to the disulfide bond in the presently disclosed spFv. In some embodiments, the anti-idiotypic antibody binds to the antigen binding domain of the presently disclosed spFv.

### 4.6. Polynucleotides, Vectors, Host Cells

The present disclosure also provides polynucleotides encoding the presently disclosed spFv. Further provided are vectors comprising such polynucleotides.

In some embodiments, the vector is an expression vector. Expression vectors may be plasmid vectors, viral vectors, vectors for baculovirus expression, vectors for prokaryotic expression, vectors for eukaryotic expression, transposon based vectors or any other vector suitable for introduction of the presently disclosed polynucleotide into a given cell or organism. The polynucleotide encoding the presently disclosed spFv may be operably linked to control sequences in the expression vector that facilitate the expression of the spFv. Such regulatory elements may include, but are not limited to, a transcriptional promoter, sequences encoding suitable mRNA ribosomal binding sites, and sequences that control the termination of transcription and translation. Expression vectors may also include one or more nontranscribed elements such as an origin of replication, other 5′ or 3′ flanking nontranscribed sequences, 5′ or 3′ nontranslated sequences (such as necessary ribosome binding sites), splice donor and acceptor sites, or selection markers. The polynucleotide may be a cDNA. The promoter driving spFv expression may be strong, weak, tissue-specific, inducible or developmental-specific promoter. Non-limiting examples of promoters include hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include, but are not limited to, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. Inducible promoters such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes and ADAR1. Vectors of the disclosure may also contain one or more Internal Ribosome Entry Site(s) (IRES). Inclusion of an IRES sequence into fusion vectors may be beneficial for enhancing expression of some proteins. Vectors of the present disclosure may be circular or linear. They may be prepared to comprise a replication system functional in a prokaryotic or eukaryotic host cell. Replication systems can be derived, e.g., from ColE1, SV40, 2μ plasmid, λ, bovine papilloma virus, and the like. The expression vectors can be designed for either transient expression, for stable expression, or for both. The expression vectors can be made for constitutive expression or for inducible expression.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Exemplary plant expression vectors include pBI01, pBI01.2, pBI121, pBI101.3, and pBIN19 (Clontech). Exemplary animal expression vectors include pEUK-C1, pMAM, and pMAMneo (Clontech). The expression vector may be a viral vector, e.g., a retroviral vector, e.g., a gamma retroviral vector.

The present disclosure also provides host cells comprising the presently disclosed vectors. In some embodiments, the host cell is a prokaryotic cell. In some embodiments, the host cell is an eukaryotic cell.

“Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, MD), CHO-K1 (ATCC CRL-61) or DG44.

The present disclosure also provides methods of producing the presently disclosed spFv. In some embodiments, the method comprises culturing a presently disclosed host cell in conditions so that the spFv is produced, and recovering the spFv produced by the host cell. Methods of making scFvs and purifying them are known. Once synthesized (either chemically or recombinantly), the spFv may be purified according to standard procedures, including, but not limited to, ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). The scFv may be substantially pure, e.g., at least from about 80% to 85% pure, at least from about 85% to 90% pure, at least from about 90% to 95% pure, or at least from about 98% to 99%, or more, pure, e.g., free from contaminants such as cell debris, macromolecules, etc. other than the subject protein

The polynucleotides encoding the spFv of the disclosure may be incorporated into vectors using standard molecular biology methods. Host cell transformation, culture, antibody expression and purification are done using well known methods.

### 4.7. Pharmaceutical Compositions and Administration

The present disclosure also provides compositions comprising the spFvs or the molecules disclosed herein. In some embodiments, the composition is a pharmaceutical composition further comprising a pharmaceutically acceptable carrier.

“Carrier” refers to a diluent, adjuvant, excipient, or vehicle with which the spFv or molecule is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may comprise pharmaceutically acceptable auxiliary substances as required to approximate physiological conditions such as pH adjusting and buffering agents, stabilizing, thickening, lubricating and coloring agents, etc. The concentration of the spFv or molecule in the composition may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration of the spFv, molecule, or composition disclosed herein may be any suitable route, including, but not limited to, parenteral administration, e.g., intradermal, intramuscular, intraperitoneal, intravenous or subcutaneous, transmucosal (oral, intranasal, intravaginal, rectal) or other means appreciated by a skilled artisan.

### 4.8. Processes for Preparing the spFv of the Present Disclosure

The present disclosure further provides processes for preparing the presently disclosed spFvs (e.g., those disclosed in Section 4.1.1). In some embodiments, the process comprises: providing a heavy chain variable region (VH) and a light chain variable region (VL) that form an antigen binding site; providing a linker (L) that comprises or is engineered to comprise a first L Cys; engineering the VH to comprise a VH Cys at a structurally conserved surface exposed VH framework residue position; and forming a disulfide bond between the VH Cys and the first L Cys to prepare a stabilized scFv.

In some embodiments, the process comprises: providing a VH and a VL that form an antigen binding site; providing a L that comprises or is engineered to comprise a second L Cys; engineering the VL to comprise a VL Cys at a structurally conserved surface exposed VL framework residue position; and forming a disulfide bond between the VL Cys and the second L Cys to prepare a stabilized scFv.

In some embodiments, the process comprises: providing a heavy chain variable region (VH) and a light chain variable region (VL) that form an antigen binding site; providing a linker (L) that comprises or is engineered to comprise a first L Cys and a second L Cys; engineering the VH to comprise a VH Cys at a structurally conserved surface exposed VH framework residue position; engineering the VL to comprise a VL Cys at a structurally conserved surface exposed VL framework residue position; and forming a disulfide bond between the VH Cys and the first L Cys and a disulfide bond between the VL Cys and the second L Cys to prepare a stabilized scFv.

In some embodiments, the disulfide bond is formed during expression of the scFv.

Any known VH/VL pair of scFv that forms an antigen binding domain may be engineered into the stabilized scFvs. Alternatively, antigen binding VH/VL pairs of interest may be identified de novo using known methods and the resulting VH/VL pairs may be engineered into spFv format.

For example, the hybridoma method of Kohler and Milstein may be used to identify VH/VL pairs that bind an antigen of interest and the resulting VH/VL pairs may be engineered as spFvs. Alternatively, transgenic animals, such as mice, rat or chicken carrying human immunoglobulin (Ig) loci in their genome may be used to generate antigen binding fragments, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://_www_kymab_com), Trianni (http://_www_trianni_com) and Ablexis (http://_www_ablexis_com) may be engaged to provide human antibodies directed against a selected antigen using technologies as described above. Phage display may also be used to generate antigen binding fragments which can be engineered as spFvs.

In some embodiments, the spFv is humanized. In some embodiments, the spFv is human. In some embodiments, the spFv is non-human.

In some embodiments, the process comprises expressing a presently disclosed polynucleotide (e.g., one disclosed in Section 4.6) in a host cell to produce a stabilized scFv.

The following examples are provided to further describe some of the embodiments disclosed herein. The examples are intended to illustrate, not to limit, the disclosed embodiments.

### 4.9. Exemplary spFvs, Molecules, and Linkers

In some embodiments, the presently disclosed spFvs or molecules comprise one or more the amino acid sequences set forth in Table 4.

TABLE 4
Protein sequence of disclosed molecules.

| | | SEQ ID |
|---|---|---|
| Molecule name | Protein sequence | NO: |
| Cris7a VL-VH | EIVLTQSPSAMSASVGDRVTITCSASSSVSYMNWYQQKPGKVPKRLIYDSSK | 125 |
| scFv | LASGVPSRFSGSGSGTEYTLTISSLQPEDFATYYCQQWSRNPPTFGQGTMLE | |
| | IKGGGGSGGGGSGGGGSGGGGSEVQLVQSGAEVKKPGESLKISCKGSGYSFT | |
| | RSTMHWVRQMPGKGLEWMGYINPSSAYTNYNQKFKDQVTISADKSISTAYLQ | |
| | WSSLKASDTAMYYCASPQVHYDYNGFPYWGQGTMVTVSSGHHHHHH | |
| Cris7a VL-VH | EIVLTQSPSAMSASVGDRVTITCSASSSVSYMNWYQQKPGCVPKRLIYDSSK | 126 |
| spFv | LASGVPSRFSGSGSGTEYTLTISSLQPEDFATYYCQQWSRNPPTFGQGTMLE | |
| | IKGGSGGSGGCPPCGSGGEVQLVQSGAEVKKPGESLKISCKGSGYSFTRSTM | |
| | HWVRQMPGKGLEWMGYINPSSAYTNYNQKFKDQVTISADKSISTAYLQWSSL | |
| | KASDTAMYYCASPQVHYDYNGFPYWGCGTMVTVSSGHHHHHH | |
| Cris7b VL-VH | EIVLTQSPSAMSASVGDRVTITCSASSSVSYMNWYQQKPGKVPKRLIYDSSK | 127 |
| scFv | LASGVPSRFSGSGSGTEYTLTISSLQPEDFATYYCQQWSRNPPTFGQGTMLE | |
| | IKGGGGSGGGGSGGGGSGGGGSQVQLLQSAAEVKKPGESLKISCKGSGYTFT | |
| | RSTMHWVRQTPGKGLEWMGYINPSSAYTNYNQKFKDQVTISADKSISTAYLQ | |
| | WSSLKASDTAMYYCARPQVHYDYNGFPYWGQGTLVTVSSGHHHHHH | |
| Cris7b VL-VH | EIVLTQSPSAMSASVGDRVTITCSASSSVSYMNWYQQKPGCVPKRLIYDSSK | 128 |
| spFv | LASGVPSRFSGSGSGTEYTLTISSLQPEDFATYYCQQWSRNPPTFGQGTMLE | |
| | IKGGSGGSGGCPPCGSGGQVQLLQSAAEVKKPGESLKISCKGSGYTFTRSTM | |
| | HWVRQTPGKGLEWMGYINPSSAYTNYNQKFKDQVTISADKSISTAYLQWSSL | |
| | KASDTAMYYCARPQVHYDYNGFPYWGCGTLVTVSSGHHHHHH | |
| BCMB749_VL | DIVMTQSQKFMSTTVGDRVSITCKASQNVGTAVAWYQQKPGQSPKLLIYSAS | 129 |
| | NRYTGVPDRFTGTGSGTDFTLTIINVQSEDLADYFCQQYGSSPWTFGGGTKL | |
| | EIK | |
| Human_CL | RTVAAPSVFIFPPSDEQLKSGTASVVCLLNNFYPREAKVQWKVDNALQSGNS | 130 |
| | QESVTEQDSKDSTYSLSSTLTLSKADYEKHKVYACEVTHQGLSSPVTKSFNR | |
| | GEC | |
| BCMB749 LC | DIVMTQSQKFMSTTVGDRVSITCKASQNVGTAVAWYQQKPGQSPKLLIYSAS | 131 |
| | NRYTGVPDRFTGTGSGTDFTLTIINVQSEDLADYFCQQYGSSPWTFGGGTKL | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |
| BCMB749_VH | QVQLQQSGAELVKPGASVKLSCKASGYTFTNNVMHWVRQKPGQGLEWIGYIL | 132 |
| | PYNDGTKYNEKFKGKATLTSDKSSSTAYMELSSLTSEDSAVYYCARYDYDGY | |
| | FDYWGQGTTLTVSS | |
| Human_HC_ | ASTKGPSVFPLAPSSKSTSGGTAALGCLVKDYFPEPVTVSWNSGALTSGVHT | 133 |
| ConstantDomains 1 | FPAVLQSSGLYSLSSVVTVPSSSLGTQTYICNVNHKPSNTKVDKKVEPKSCD | |
| | KTHTCPPCPAPEAAGGPSVFLFPPKPKDTLMISRTPEVTCVVVSVSHEDPEV | |
| | KFNWYVDGVEVHNAKTKPREEQYNSTYRVVSVLTVLHQDWLNGKEYKCKVSN | |
| | KALPAPIEKTISKAKGQPREPQVYTLPPSREEMTKNQVSLSCAVKGFYPSDI | |
| | AVEWESNGQPENNYKTTPPVLDSDGSFFLVSKLTVDKSRWQQGNVFSCSVMH | |
| | EALHNRFTQKSLSLSPGK | |
| BCMB749 HC1 | QVQLQQSGAELVKPGASVKLSCKASGYTFTNNVMHWVRQKPGQGLEWIGYIL | 134 |
| | PYNDGTKYNEKFKGKATLTSDKSSSTAYMELSSLTSEDSAVYYCARYDYDGY | |
| | FDYWGQGTTLTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKTHTCPPCPAPEAAGGPSVFLFPPKPKDTLMISRTPEV | |
| | TCVVVSVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSREEMTKNQV | |
| | SLSCAVKGFYPSDIAVEWESNGQPENNYKTTPPVLDSDGSFFLVSKLTVDKS | |
| | RWQQGNVFSCSVMHEALHNRFTQKSLSLSPGK | |
| BCMB749h_VL | DIQMTQSPSSVSASVGDRVTITCKASQNVGTAVAWYQQKPGQSPKLLIYSAS | 135 |
| | NRYTGVPSRFSGSGSGTDFTLTISSLQPEDFATYFCQQYGSSPWTFGGGTKV | |
| | EIK | |
| BCMB749h LC | DIQMTQSPSSVSASVGDRVTITCKASQNVGTAVAWYQQKPGQSPKLLIYSAS | 136 |
| | NRYTGVPSRFSGSGSGTDFTLTISSLQPEDFATYFCQQYGSSPWTFGGGTKV | |
| | EIKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKS | |
| | FNRGEC | |
| BCMB749h VH | QVQLVQSGAEVKKPGASVKVSCKASGYTFTNNVMHWVRQAPGQGLEWMGYIL | 137 |
| | PYNDGTKYNQKFQGRVTLTSDKSASTAYMELSSLRSEDTAVYYCARYDYDGY | |
| | FDYWGQGTTVTVSS | |
| BCMB749h HC1 | QVQLVQSGAEVKKPGASVKVSCKASGYTFTNNVMHWVRQAPGQGLEWMGYIL | 138 |
| | PYNDGTKYNQKFQGRVTLTSDKSASTAYMELSSLRSEDTAVYYCARYDYDGY | |
| | FDYWGQGTTVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPS | |
| | NTKVDKKVEPKSCDKTHTCPPCPAPEAAGGPSVFLFPPKPKDTLMISRTPEV | |
| | TCVVVSVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSREEMTKNQV | |
| | SLSCAVKGFYPSDIAVEWESNGQPENNYKTTPPVLDSDGSFFLVSKLTVDKS | |
| | RWQQGNVFSCSVMHEALHNRFTQKSLSLSPGK | |
| Human_HC_ | EPKSCDKTHTCPPCPAPEAAGGPSVFLFPPKPKDTLMISRTPEVTCVVVSVS | 139 |
| ConstantDomains 2 | HEDPEVKFNWYVDGVEVHNAKTKPREEQYNSTYRVVSVLTVLHQDWLNGKEY | |
| | KCKVSNKALPAPIEKTISKAKGQPREPQVYTLPPSRDELTKNQVSLWCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| Cris7b VL-VH | EIVLTQSPSAMSASVGDRVTITCSASSSVSYMNWYQQKPGKVPKRLIYDSSK | 140 |
| scFv HC2 | LASGVPSRFSGSGSGTEYTLTISSLQPEDFATYYCQQWSRNPPTFGQGTMLE | |
| | IKGGSEGKSSGSGSESKSTGGSQVQLLQSAAEVKKPGESLKISCKGSGYTFT | |
| | RSTMHWVRQTPGKGLEWMGYINPSSAYTNYNQKFKDQVTISADKSISTAYLQ | |
| | WSSLKASDTAMYYCARPQVHYDYNGFPYWGCGTLVTVSSEPKSCDKTHTCPP | |
| | CPAPEAAGGPSVFLFPPKPKDTLMISRTPEVTCVVVSVSHEDPEVKFNWYVD | |
| | GVEVHNAKTKPREEQYNSTYRVVSVLTVLHQDWLNGKEYKCKVSNKALPAPI | |
| | EKTISKAKGQPREPQVYTLPPSRDELTKNQVSLWCLVKGFYPSDIAVEWESN | |
| | GQPENNYKTTPPVLDSDGSFFLYSKLTVDKSRWQQGNVFSCSVMHEALHNHY | |
| | TQKSLSLSPGK | |
| Cris7b VL-VH | EIVLTQSPSAMSASVGDRVTITCSASSSVSYMNWYQQKPGCVPKRLIYDSSK | 141 |
| spFv HC2 | LASGVPSRFSGSGSGTEYTLTISSLQPEDFATYYCQQWSRNPPTFGQGTMLE | |
| | IKGGGSGGSGGCPPCGGSGGQVQLLQSAAEVKKPGESLKISCKGSGYTFTRS | |
| | TMHWVRQTPGKGLEWMGYINPSSAYTNYNQKFKDQVTISADKSISTAYLQWS | |
| | SLKASDTAMYYCARPQVHYDYNGFPYWGCGTLVTVSSEPKSCDKTHTCPPCP | |
| | APEAAGGPSVFLFPPKPKDTLMISRTPEVTCVVVSVSHEDPEVKFNWYVDGV | |
| | EVHNAKTKPREEQYNSTYRVVSVLTVLHQDWLNGKEYKCKVSNKALPAPIEK | |
| | TISKAKGQPREPQVYTLPPSRDELTKNQVSLWCLVKGFYPSDIAVEWESNGQ | |
| | PENNYKTTPPVLDSDGSFFLYSKLTVDKSRWQQGNVFSCSVMHEALHNHYTQ | |
| | KSLSLSPGK | |
| CD3B219a99v | QTVVTQEPSLTVSPGGTVTLTCRSSTGAVTTSNYANWVQQKPGQAPRGLIGG | 142 |
| scFv HC2 | TNKRAPGTPARFSGSLLGGKAALTLSGVQPEDEAEYYCALWYSNLWVFGGGT | |
| | KLTVLGGSEGKSSGSGSESKSTGGSEVQLVESGGGLVQPGGSLRLSCAASGF | |
| | TFNTYAMNWVRQAPGKGLEWVARIRSKYNNYATYYAASVKGRFTISRDDSKN | |
| | SLYLQMNSLKTEDTAVYYCVRHGNFGNSYVSWFAYWGQGTLVTVSSEPKSCD | |
| | KTHTCPPCPAPEAAGGPSVFLFPPKPKDTLMISRTPEVTCVVVSVSHEDPEV | |
| | KFNWYVDGVEVHNAKTKPREEQYNSTYRVVSVLTVLHQDWLNGKEYKCKVSN | |
| | KALPAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFSCSVMH | |
| | EALHNHYTQKSLSLSPGK | |
| CD3B219a99v | QTVVTQEPSLTVSPGGTVTLTCRSSTGAVTTSNYANWVQQKPGCAPRGLIGG | 143 |
| spFv HC2 | TNKRAPGTPARFSGSLLGGKAALTLSGVQPEDEAEYYCALWYSNLWVFGGGT | |
| | KLTVLGGGSGGSGGCPPCGGSGGEVQLVESGGGLVQPGGSLRLSCAASGFTF | |
| | NTYAMNWVRQAPGKGLEWVARIRSKYNNYATYYAASVKGRFTISRDDSKNSL | |
| | YLQMNSLKTEDTAVYYCVRHGNFGNSYVSWFAYWGCGTLVTVSSEPKSCDKT | |
| | HTCPPCPAPEAAGGPSVFLFPPKPKDTLMISRTPEVTCVVVSVSHEDPEVKF | |
| | NWYVDGVEVHNAKTKPREEQYNSTYRVVSVLTVLHQDWLNGKEYKCKVSNKA | |
| | LPAPIEKTISKAKGQPREPQVYTLPPSRDELTKNQVSLWCLVKGFYPSDIAV | |
| | EWESNGQPENNYKTTPPVLDSDGSFFLYSKLTVDKSRWQQGNVFSCSVMHEA | |
| | LHNHYTQKSLSLSPGK | |

In some embodiments, the “Cris7a VL-VH scFv” comprises the amino acid sequence of SEQ ID NO: 125.

In some embodiments, the “Cris7a VL-VH spFv” comprises the amino acid sequence of SEQ ID NO: 126.

In some embodiments, the “Cris7b VL-VH scFv” the amino acid sequence of SEQ ID NO: 127. In some embodiments, the “Cris7b VL-VH spFv” comprises the amino acid sequence of SEQ ID NO: 128. In some embodiments, the “BCMB749_VL” comprises the amino acid sequence of SEQ ID NO: 129.

In some embodiments, the “Human CL” comprises the amino acid sequence of SEQ ID NO: 130.

In some embodiments, the “BCMB749 LC” comprises the amino acid sequence of SEQ ID NO: 131.

In some embodiments, the “BCMB749_VH” of comprises the amino acid sequence of SEQ ID NO: 132.

In some embodiments, the “Human_HC_ConstantDomains 1” comprises the amino acid sequence of SEQ ID NO: 133

In some embodiments, the “BCMB749 HC1” comprises the amino acid sequence of SEQ ID NO: 134.

In some embodiments, the “BCMB749h_VL” comprises the amino acid sequence of SEQ ID NO: 135.

In some embodiments, the “BCMB749h LC” comprises the amino acid sequence of SEQ ID NO: 136.

In some embodiments, the “BCMB749h_VH” comprises the amino acid sequence of SEQ ID NO: 137.

In some embodiments, the “BCMB749h HC1” comprises the amino acid sequence of SEQ ID NO: 138.

In some embodiments, the “Human_HC_ConstantDomains 2” comprises the amino acid sequence of SEQ ID NO: 139.

In some embodiments, the “Cris7b VL-VH scFv HC2” comprises the amino acid sequence of SEQ ID NO: 140.

In some embodiments, the “Cris7b VL-VH spFv HC2” comprises the amino acid sequence of SEQ ID NO: 141.

In some embodiments, the “CD3B219a99v scFv HC2” comprises the amino acid sequence of SEQ ID NO: 142.

In some embodiments, the “CD3B219a99v spFv HC2” comprises the amino acid sequence of SEQ ID NO: 143.

In further embodiments, the scFv linker of the disclosure comprises the amino acid sequences set forth in Table 5.

TABLE 5
Protein sequence of scFv and spFv linker.

| | Linker | | SEQ ID |
|---|---|---|---|
| Molecule name | type | Protein Sequence | NO: |
| GLk1 scFv VL-VH | 4× G4S | GGGGSGGGGSGGGGSGGGGS | 2 |
| GLk1 spFv VL-VH | 9 + 4 + 5 | GGGSGGSGGCPPCGGSGG | 3 |
| GLk1 scFv VH-VL | 4× G4S | GGGGSGGGGSGGGGSGGGGS | 2 |
| GLk1 spFv VH-VL | 9 + 4 + 5 | GGGSGGSGGCPPCGGSGG | 3 |
| GLk2 scFv VL-VH | 4× G4S | GGGGSGGGGSGGGGSGGGGS | 2 |
| GLk2 spFv VL-VH | 9 + 4 + 5 | GGGSGGSGGCPPCGGSGG | 3 |
| GLk2 scFv VH-VL | 4× G4S | GGGGSGGGGSGGGGSGGGGS | 2 |
| GLk2 spFv VH-VL | 6 + 4 + 6 | GGGSGGCPPCGGGSGG | 4 |
| CAT2200a scFv VL-VH | 4× G4S | GGGGSGGGGSGGGGSGGGGS | 2 |
| CAT2200a spFv VL-VH | 8 + 4 + 4 | GGSGGSGGCPPCGSGG | 5 |
| CAT2200b scFv VL-VH | 4× G4S | GGGGSGGGGSGGGGSGGGGS | 2 |
| CAT2200a spFv VL-VH | 9 + 4 + 4 | GGGSGGSGGCPPCGSGG | 6 |
| CAT2200a scFv VH-VL | 4× G4S | GGGGSGGGGSGGGGSGGGGS | 2 |
| CAT2200b spFv VH-VL | 9 + 4 + 4v2 | GGGSGGGSGCPPCGGGG | 7 |

In some embodiments, the “GLk1 scFv VL-VH” linker comprises the amino acid sequence of SEQ ID NO:2

In some embodiments, the “GLk1 spFv VL-VH” linker comprises the amino acid sequence of SEQ ID NO: 3.

In some embodiments, the “GLk1 scFv VH-VL” linker comprises the amino acid sequence of SEQ ID NO:2.

In some embodiments, the “GLk1 spFv VH-VL” linker comprises the amino acid sequence of SEQ ID NO:3.

In some embodiments, the “GLk2 scFv VL-VH” linker comprises the amino acid sequence of SEQ ID NO:2.

In some embodiments, the “GLk2 spFv VL-VH” linker comprises the amino acid sequence of SEQ ID NO:3.

In some embodiments, the “GLk2 scFv VH-VL” linker comprises the amino acid sequence of SEQ ID NO:2.

In some embodiments, the “GLk2 spFv VH-VL” linker comprises the amino acid sequence of SEQ ID NO:4.

In some embodiments, the “CAT2200a scFv VL-VH” linker of the disclosure comprises the amino acid sequence of SEQ ID NO:2.

In some embodiments, the “CAT2200a spFv VL-VH” linker of the disclosure comprises the amino acid sequence of SEQ ID NO:5.

In some embodiments, the “CAT2200b scFv VL-VH” linker of the disclosure comprises the amino acid sequence of SEQ ID NO:2.

In some embodiments, the “CAT2200a spFv VL-VH” linker of the disclosure comprises the amino acid sequence of SEQ ID NO:6.

In some embodiments, the “CAT2200a scFv VH-VL” linker of the disclosure comprises the amino acid sequence of SEQ ID NO:2.

In some embodiments, the “CAT2200b spFv VH-VL” linker of the disclosure comprises the amino acid sequence of SEQ ID NO:7.

### 5. EMBODIMENTS

This invention provides the following non-limiting embodiments.

In one set of embodiments, provided are:

A1. A molecule comprising an antigen-binding fragment (Fab), a single chain variable fragment (scFv), and a fragment crystallizable region (Fc region), wherein the scFv comprises a heavy chain variable region (VH), a linker (L), and a light chain variable region (VL), wherein the scFv comprises:
a) a disulfide bond between a structurally conserved surface exposed VH position which is mutated to cysteine (Cys) and a L Cys;
b) a disulfide bond between a structurally conserved surface exposed VL position which is mutated to Cys and a L Cys; or
c) a first disulfide bond between a structurally conserved surface exposed VH Cys and a first L Cys and a second disulfide bond between a structurally conserved surface exposed VL Cys and a second L Cys, optionally wherein the molecule has improved stability, expression yields and/or quality as compared to a comparable molecule absent a disulfide bond, such as a comparable molecule absent the first disulfide bond and the second disulfide bond.

A2. The molecule of embodiment A1, wherein
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

A3. The molecule of embodiment A1 or A2, wherein the distance between the VH Cys and the VL Cys is from about 5 Å to about 10 Å or from about 7 Å to about 9 Å.

A4. The molecule of any one of embodiments A1-A3, wherein the VH Cys is at H3, H5, H40, H43, H46 or H105, wherein the residue numbering is according to Chothia.

A5. The molecule of any one of embodiments A1-A4, wherein the VL Cys is at L3, L5, L39, L42, L43, L45, L100 or L102, wherein the residue numbering is according to Chothia.

A6. The molecule of any one of embodiments A1-A5, wherein
a) the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
jj) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102, or
qq) the VH Cys is at H105 and the VL Cys is at L43, wherein the residue numbering is according to Chothia.

A7. The molecule of any one of embodiments A1-A6, wherein the L comprises a contiguous amino acid sequence derived from an immunoglobulin (Ig) hinge region.

A8. The molecule of any one of embodiments A1-A7, wherein the Ig hinge region is derived from a human Ig hinge region or a non-human Ig hinge region.

A9. The molecule of any one of embodiments A1-A8, wherein the Ig hinge region is derived from a human Ig hinge region.

A10. The molecule of any one of embodiments A1-A9, wherein the human Ig hinge region is an IgG1, IgG2, IgG3, or IgG4 isotype.

A11. The molecule of any one of embodiments A1-A10, wherein the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

A12. The molecule of embodiment A11, wherein the L comprises an amino acid sequence C(X)_{y}C (SEQ ID NO: 24), wherein X is Gly, Ser or Pro, and y is an integer from 1 to 3.

A13. The molecule of any one of embodiments A1-A12, wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

A14. The molecule of any one of embodiments A1-A13, wherein the L comprises from about 14 to about 19 amino acids, such as about 14, about 15, about 16, about 17, about 18 or about 19 amino acids; and/or the L has a length of from about 14 to about 19 amino acids, such as about 14, about 15, about 16, about 17, about 18 or about 19 amino acids.

A15. The molecule of any one of embodiments A1-A14 wherein the L comprises the amino acid sequence (X)ₘC(X)_{y}C(X)ₙ(SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

A16. The molecule of embodiment A15, wherein the L comprises the amino acid sequence (X)ₘC(X)_{y}C(X)ₙ(SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

A17. The molecule of embodiment A16, wherein the L comprises the amino acid sequence (X)ₘC(X)_{y}C(X)ₙ(SEQ ID NO: 27); wherein X is Gly or Pro, m is an integer from 6 to 9, y is an integer from 1 to 3 and n is an integer from 4 to 6.

A18. The molecule of any one of embodiments A1-A17, wherein the L comprises the amino acid sequence of SEQ ID NO: 2, SEQ ID NO: 3, SEQ ID NO: 4, SEQ ID NO: 5, SEQ ID NO: 6, or SEQ ID NO: 7.

A19. The molecule of any one of embodiments A1-A18, wherein the scFv is in the VL-L-VH orientation.

A20. The molecule of any one of embodiments A1-A18, wherein the scFv is in the VH-L-VL orientation.

A21. The molecule of any one of embodiments A1-A19, wherein
a) the VH comprises a Cys at H105;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A22. The molecule of any one of embodiments A1-A19, wherein
a) the VH comprises a Cys at H105;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A23. The molecule of any one of embodiments A1-A19, wherein
a) the VH comprises a Cys at H105;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A24. The molecule of any one of embodiments A1-A19, wherein
a) the VH comprises a Cys at H5;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A25. The molecule of any one of embodiments A1-A19, wherein
a) the VH comprises a Cys at H5;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A26. The molecule of any one of embodiments A1-A19, wherein
a) the VH comprises a Cys at H5;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A27. The molecule of any one of embodiments A1-A19, wherein
a) the VH comprises a Cys at H3;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A28. The molecule of any one of embodiments A1-A19, wherein
a) the VH comprises a Cys at H3;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A29. The molecule of any one of embodiments A1-A19, wherein
a) the VH comprises a Cys at H3;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A30. The molecule of any one of embodiments A1-A18 and A20, wherein
a) the VH comprises a Cys at H43;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A31. The molecule of any one of embodiments A1-A18 and A20, wherein
a) the VH comprises a Cys at H43;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A32. The molecule of any one of embodiments A1-A18 and A20, wherein
a) the VH comprises a Cys at H43;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A33. The molecule of any one of embodiments A1-A18 and A20, wherein
a) the VH comprises a Cys at H43;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A34. The molecule of any one of embodiments A1-A18 and A20, wherein
a) the VH comprises a Cys at H40;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A35. The molecule of any one of embodiments A1-A18 and A20, wherein
a) the VH comprises a Cys at H40;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A36. The molecule of any one of embodiments A1-A18 and A20, wherein
a) the VH comprises a Cys at H40;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A37. The molecule of any one of embodiments A1-A18 and A20, wherein
a) the VH comprises a Cys at H40;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A38. The molecule of any one of embodiments A1-A18 and A20, wherein
a) the VH comprises a Cys at H46;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A39. The molecule of any one of embodiments A1-A18 and A20, wherein
a) the VH comprises a Cys at H46;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A40. The molecule of any one of embodiments A1-A18 and A20, wherein
a) the VH comprises a Cys at H46;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A41. The molecule of any one of embodiments A1-A18 and A20, wherein
a) the VH comprises a Cys at H46;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

A42. The molecule of any one of embodiments A21-A41, wherein the L comprises the amino acid sequence of SEQ ID NO: 3.

A43. The molecule of any one of embodiments A21-A41, wherein the L comprises the amino acid sequence of SEQ ID NO: 6.

A44. The molecule of any one of embodiments A21-A41, wherein the L comprises the amino acid sequence of SEQ ID NO: 7.

A45. The molecule of any one of embodiments A1-A44, wherein the binding molecules comprises a heavy chain, a light chain and a polypeptide,
wherein the N-terminus of the heavy chain and the light chain form the Fab;
wherein the polypeptide comprises the scFv at the N-terminus; and
wherein the C-terminus of the polypeptide and the C-terminus of the heavy chain form the Fc region.

A46. The molecule of any one of embodiments A1 to A45, wherein the Fab binds to a tumor antigen and the scFv binds to a T cell antigen; and wherein optionally the tumor antigen is BCMA and the T cell antigen is CD3.

A47. The molecule of embodiment A46, wherein the scFv comprises the amino acid sequence of SEQ ID NO: 126 or SEQ ID NO: 128.

A48. The molecule of embodiment A46 or embodiment A47, wherein (i) the Fab comprises a VH comprising the amino acid sequence of SEQ ID NO: 132, and a VL comprising the amino acid sequence of SEQ ID NO: 129; or (ii) the Fab comprises a VH comprising the amino acid sequence of SEQ ID NO: 137, and a VL comprising the amino acid sequence of SEQ ID NO: 135.

A49. The molecule of embodiment A47 or embodiment A48, wherein
a) the VH comprises a Cys at H105;
b) the VL comprises a Cys at L43;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

A50. A polynucleotide encoding the molecule of any one of embodiments A1-A49 or a fragment or a polypeptide thereof.

A51. A vector comprising the polynucleotide of embodiment A50.

A52. A host cell comprising the vector of embodiment A51.

A53. A method of producing a binding molecule, comprising culturing the host cell of embodiment A52 in conditions so that the molecule is produced, and purifying the binding molecule.

A54. The method of embodiment A53, wherein the host cell is a prokaryotic cell.

A55. The method of embodiment A53, wherein the host cell is an eukaryotic cell.

In one set of embodiments, provided are:

B1. A composition comprising a molecule of any one of embodiments A1-A49, optionally wherein the composition is a pharmaceutical composition further comprising a pharmaceutically acceptable excipient, optionally wherein the molecule comprises an antigen-binding fragment (Fab), a single chain variable fragment (scFv), and a fragment crystallizable region (Fc region), wherein the scFv comprises a heavy chain variable region (VH), a linker (L) and a light chain variable region (VL), wherein the scFv comprises:
a) a disulfide bond between a structurally conserved surface exposed VH cysteine (Cys) and a L Cys;
b) a disulfide bond between a structurally conserved surface exposed VL Cys and a L Cys; or
c) a first disulfide bond between a structurally conserved surface exposed VH Cys and a first L Cys and a second disulfide bond between a structurally conserved surface exposed VL Cys and a second L Cys.

B2. The composition of embodiment B1, wherein
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

B3. The composition of embodiment B1 or B2, wherein the distance between the VH Cys and the VL Cys is from about 5 Å to about 10 Å or from about 7 Å to about 9 Å.

B4. The composition of any one of embodiments B1-B3, wherein the VH Cys is at H3, H5, H40, H43, H46 or H105, wherein the residue numbering is according to Chothia.

B5. The composition of any one of embodiments B1-B4, wherein the VL Cys is at L3, L5, L39, L42, L43, L45, L100 or L102, wherein the residue numbering is according to Chothia.

B6. The composition of any one of embodiments B1-B5, wherein
a) the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
jj) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102,
qq) the VH Cys is at H105 and the VL Cys is at L43,
   wherein the residue numbering is according to Chothia.

B7. The composition of any one of embodiments B1-B6, wherein the L comprises a contiguous amino acid sequence derived from an immunoglobulin (Ig) hinge region.

B8. The composition any one of embodiments B1-B7, wherein the Ig hinge region is derived from a human Ig hinge region or a non-human Ig hinge region.

B9. The composition of any one of embodiments B1-B8, wherein the Ig hinge region is derived from a human Ig hinge region.

B10. The composition of any one of embodiments B1-B9, wherein the human Ig hinge region is an IgG1, IgG2, IgG3, or IgG4 isotype.

B11. The composition of any one of embodiments B1-B10, wherein the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

B12. The composition of embodiment B11, wherein the L comprises an amino acid sequence C(X)_{y}C (SEQ ID NO: 24), wherein X is Gly, Ser or Pro, and y is an integer from 1 to 3.

B13. The composition of any one of embodiments B1-B12, wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

B14. The composition of any one of embodiments B1-B13, wherein the L comprises from about 14 to about 19 amino acids, such as about 14, about 15, about 16, about 17, about 18 or about 19 amino acids.

B15. The composition of any one of embodiments B1-B14 wherein the L comprises the amino acid sequence (X)ₘC(X)_{y}C(X)ₙ(SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

B16. The composition of embodiment B15, wherein the L comprises the amino acid sequence (X)ₘC(X)_{y}C(X)ₙ(SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

B17. The composition of embodiment B16, wherein the L comprises the amino acid sequence (X)ₘC(X)_{y}C(X)ₙ(SEQ ID NO: 27); wherein X is Gly or Pro, m is an integer from 6 to 9, y is an integer from 1 to 3 and n is an integer from 4 to 6.

B18. The composition of any one of embodiments B1-B17, wherein the L comprises the amino acid sequence of SEQ ID NO: 2, SEQ ID NO: 3, SEQ ID NO: 4, SEQ ID NO: 5, SEQ ID NO: 6, or SEQ ID NO: 7.

B19. The composition of any one of embodiments B1-B18, wherein the scFv is in the VL-L-VH orientation.

B20. The composition of any one of embodiments B1-B18, wherein the scFv is in the VH-L-VL orientation.

B21. The composition of any one of embodiments B1-B19, wherein
a) the VH comprises a Cys at H105;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B22. The composition of any one of embodiments B1-B19, wherein
a) the VH comprises a Cys at H105;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B23. The composition of any one of embodiments B1-B19, wherein
a) the VH comprises a Cys at H105;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B24. The composition of any one of embodiments B1-B19, wherein
a) the VH comprises a Cys at H5;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B25. The composition of any one of embodiments B1-B19, wherein
a) the VH comprises a Cys at H5;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B26. The composition of any one of embodiments B1-B19, wherein
a) the VH comprises a Cys at H5;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B27. The composition of any one of embodiments B1-B19, wherein
a) the VH comprises a Cys at H3;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B28. The composition of any one of embodiments B1-B19, wherein
a) the VH comprises a Cys at H3;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B29. The composition of any one of embodiments B1-B19, wherein
a) the VH comprises a Cys at H3;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

B30. The composition of any one of embodiments B1-B18 and B20, wherein
a) the VH comprises a Cys at H43;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B31. The composition of any one of embodiments B1-B18 and B20, wherein
a) the VH comprises a Cys at H43;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B32. The composition of any one of embodiments B1-B18 and B20, wherein
a) the VH comprises a Cys at H43;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B33. The composition of any one of embodiments B1-B18 and B20, wherein
a) the VH comprises a Cys at H43;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B34. The composition of any one of embodiments B1-B18 and B20, wherein
a) the VH comprises a Cys at H40;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B35. The composition of any one of embodiments B1-B18 and B20, wherein
a) the VH comprises a Cys at H40;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B36. The composition of any one of embodiments B1-B18 and B20, wherein
a) the VH comprises a Cys at H40;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B37. The composition of embodiment B1, wherein
a) the VH comprises a Cys at H40;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B38. The composition of any one of embodiments B1-B18 and B20, wherein
a) the VH comprises a Cys at H46;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B39. The composition of any one of embodiments B1-B18 and B20, wherein
a) the VH comprises a Cys at H46;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B40. The composition of any one of embodiments B1-B18 and B20, wherein
a) the VH comprises a Cys at H46;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B41. The composition of any one of embodiments B1-B18 and B20, wherein
a) the VH comprises a Cys at H46;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

B42. The composition of any one of embodiments B21-B41, wherein the L comprises the amino acid sequence of SEQ ID NO: 3.

B43. The composition of any one of embodiments B21-B41, wherein the L comprises the amino acid sequence of SEQ ID NO: 6.

B44. The composition of any one of embodiments B21-B41, wherein the L comprises the amino acid sequence of SEQ ID NO: 7.

B45. The composition of any one of embodiments B1-B43, wherein the binding molecules comprises a heavy chain, a light chain and a polypeptide,
wherein the N-terminus of the heavy chain and the light chain form the Fab;
wherein the polypeptide comprises the scFv at the N-terminus; and
wherein the C-terminus of the polypeptide and the C-terminus of the heavy chain form the Fc region.

B46. The composition of any one of embodiments B1 to B45, wherein the Fab binds to a tumor antigen and the scFv binds to a T cell antigen; and wherein optionally the tumor antigen is BCMA and the T cell antigen is CD3.

B47. The composition of embodiment B46, wherein the scFv comprises the amino acid sequence of SEQ ID NO: 126 or SEQ ID NO: 128.

B48. The composition of embodiment B46 or embodiment B47, wherein (i) the Fab comprises a VH comprising the amino acid sequence of SEQ ID NO: 132, and a VL comprising the amino acid sequence of SEQ ID NO: 129; or (ii) the Fab comprises a VH comprising the amino acid sequence of SEQ ID NO: 137, and a VL comprising the amino acid sequence of SEQ ID NO: 135.

B49. The composition of embodiment B47 or embodiment B48, wherein
a) the VH comprises a Cys at H105;
b) the VL comprises a Cys at L43;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

In one set of embodiments, provided are:

C1. A method of producing a binding molecule, comprising introducing a polynucleotide encoding the molecule or a fragment thereof into a host cell; culturing the host cell in conditions so that the molecule is produced, and purifying the binding molecule, wherein the molecule comprises an antigen-binding fragment (Fab), a single chain variable fragment (scFv), and a fragment crystallizable region (Fc region), wherein the scFv comprises a heavy chain variable region (VH), a linker (L) and a light chain variable region (VL), wherein the scFv comprises:
a) a disulfide bond between a structurally conserved surface exposed VH cysteine (Cys) and a L Cys;
b) a disulfide bond between a structurally conserved surface exposed VL Cys and a L Cys; or
c) a first disulfide bond between the structurally conserved surface exposed VH Cys and a first L Cys and a second disulfide bond between the structurally conserved surface exposed VL Cys and a second L Cys.

C2. The method of embodiment C1, wherein
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

C3. The method of embodiment C1 or C2, wherein the distance between the VH Cys and the VL Cys is from about 5 Å to about 10 Å or from about 7 Å to about 9 Å.

C4. The method of any one of embodiments C1-C3, wherein the VH Cys is at H3, H5, H40, H43, H46, or H105, wherein the residue numbering is according to Chothia.

C5. The method of any one of embodiments C1-C4, wherein the VL Cys is at L3, L5, L39, L42, L43, L45, L100, or L102, wherein the residue numbering is according to Chothia.

C6. The method of any one of embodiments C1-C5, wherein
a) the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
jj) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102,
qq) the VH Cys is at H105 and the VL Cys is at L43,
   wherein the residue numbering is according to Chothia.

C7. The method of any one of embodiments C1-C6, wherein the L comprises a contiguous amino acid sequence derived from an immunoglobulin (Ig) hinge region.

C8. The method of any one of embodiments C1-C7, wherein the Ig hinge region is derived from a human Ig hinge region or a non-human Ig hinge region.

C9. The method of any one of embodiments C1-C8, wherein the Ig hinge region is derived from a human Ig hinge region.

C10. The method of embodiment C9, wherein the human Ig hinge region is an IgG1, IgG2, IgG3, or IgG4 isotype.

C11. The method of any one of embodiments C1-C10, wherein the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

C12. The method of embodiment C11, wherein the L comprises an amino acid sequence C(X)_{y}C (SEQ ID NO: 24), wherein X is Gly, Ser or Pro, and y is an integer from 1 to 3.

C13. The method of any one of embodiments C1-C12, wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

C14. The method of any one of embodiments C1-C13, wherein the L comprises from about 14 to about 19 amino acids, such as about 14, about 15, about 16, about 17, about 18 or about 19 amino acids; and/or the L has a length of from about 14 to about 19 amino acids, such as about 14, about 15, about 16, about 17, about 18 or about 19 amino acids.

C15. The method of any one of embodiments C1-C14 wherein the L comprises the amino acid sequence (X)ₘC(X)_{y}C(X)ₙ(SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

C16. The method of embodiment C15, wherein the L comprises the amino acid sequence (X)ₘC(X)_{y}C(X)ₙ(SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

C17. The method of embodiment C16, wherein the L comprises the amino acid sequence (X)ₘC(X)_{y}C(X)ₙ(SEQ ID NO: 27); wherein X is Gly or Pro, m is an integer from 6 to 9, y is an integer from 1 to 3 and n is an integer from 4 to 6.

C18. The method of any one of embodiments C1-C17, wherein the L comprises the amino acid sequence of SEQ ID NO: 2, SEQ ID NO: 3, SEQ ID NO: 4, SEQ ID NO: 5, SEQ ID NO: 6, or SEQ ID NO: 7.

C19. The method of any one of embodiments C1-C18, wherein the scFv is in the VL-L-VH orientation.

C20. The method of any one of embodiments C1-C18, wherein the scFv is in the VH-L-VL orientation.

C21. The method of any one of embodiments C1-C19, wherein
a) the VH comprises a Cys at H105;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C22. The method of any one of embodiments C1-C19, wherein
a) the VH comprises a Cys at H105;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C23. The method of any one of embodiments C1-C19, wherein
a) the VH comprises a Cys at H105;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C24. The method of any one of embodiments C1-C19, wherein
a) the VH comprises a Cys at H5;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C25. The method of any one of embodiments C1-C19, wherein
a) the VH comprises a Cys at H5;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C26. The method of any one of embodiments C1-C19, wherein
a) the VH comprises a Cys at H5;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C27. The method of any one of embodiments C1-C19, wherein
a) the VH comprises a Cys at H3;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C28. The method of any one of embodiments C1-C19, wherein
a) the VH comprises a Cys at H3;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C29. The method of any one of embodiments C1-C19, wherein

a) the VH comprises a Cys at H3;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C30. The method of any one of embodiments C1-C18 and C20, wherein
a) the VH comprises a Cys at H43;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C31. The method of any one of embodiments C1-C18 and C20, wherein
a) the VH comprises a Cys at H43;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C32. The method of any one of embodiments C1-C18 and C20, wherein
a) the VH comprises a Cys at H43;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C33. The method of any one of embodiments C1-C18 and C20, wherein
a) the VH comprises a Cys at H43;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C34. The method of any one of embodiments C1-C18 and C20, wherein
a) the VH comprises a Cys at H40;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C35. The method of any one of embodiments C1-C18 and C20, wherein
a) the VH comprises a Cys at H40;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C36. The method of any one of embodiments C1-C18 and C20, wherein
a) the VH comprises a Cys at H40;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C37. The method of any one of embodiments C1-C18 and C20, wherein
a) the VH comprises a Cys at H40;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C38. The method of any one of embodiments C1-C18 and C20, wherein
a) the VH comprises a Cys at H46;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C39. The method of any one of embodiments C1-C18 and C20, wherein
a) the VH comprises a Cys at H46;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C40. The method of any one of embodiments C1-C18 and C20, wherein
a) the VH comprises a Cys at H46;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C41. The method of any one of embodiments C1-C18 and C20, wherein
a) the VH comprises a Cys at H46;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

C42. The method of any one of embodiments C21-C41, wherein the L comprises the amino acid sequence of SEQ ID NO: 3.

C43. The method of any one of embodiments C21-C41, wherein the L comprises the amino acid sequence of SEQ ID NO: 6.

C44. The method of any one of embodiments C21-C41, wherein the L comprises the amino acid sequence of SEQ ID NO: 7.

C45. The method of embodiment C1, wherein the binding molecules comprises a heavy chain, a light chain and a polypeptide,
wherein the N-terminus of the heavy chain and the light chain form the Fab;
wherein the polypeptide comprises the scFv at the N-terminus; and
wherein the C-terminus of the polypeptide and the C-terminus of the heavy chain form the Fc region.

C46. The method of any one of embodiments C1 to C45, wherein the Fab binds to a tumor antigen and the scFv binds to a T cell antigen; and wherein optionally the tumor antigen is BCMA and the T cell antigen is CD3.

C47. The method of embodiment C46, wherein the scFv comprises the amino acid sequence of SEQ ID NO: 126 or SEQ ID NO: 128.

C48. The method of embodiment C46 or embodiment C47, wherein (i) the Fab comprises a VH comprising the amino acid sequence of SEQ ID NO: 132, and a VL comprising the amino acid sequence of SEQ ID NO: 129; or (ii) the Fab comprises a VH comprising the amino acid sequence of SEQ ID NO: 137, and a VL comprising the amino acid sequence of SEQ ID NO: 135.

C49. The method of embodiment C47 or embodiment C48, wherein
a) the VH comprises a Cys at H105;
b) the VL comprises a Cys at L43;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

C50. The method of any one of embodiments C1-C49, wherein the host cell is a prokaryotic cell.

C51. The method of any one of embodiments C1-C49, wherein the host cell is an eukaryotic cell.

In one set of embodiments, provided are:

D1. A method for directing or engaging a cell to a target cell, comprising contacting the target cell with a binding molecule,
wherein the molecule comprises an antigen-binding fragment (Fab), a single chain variable fragment (scFv), and a fragment crystallizable region (Fc region), wherein the scFv comprises a heavy chain variable region (VH), a linker (L), and a light chain variable region (VL), wherein the scFv comprises:
a) a disulfide bond between a structurally conserved surface exposed VH cysteine (Cys) and a L Cys;
b) a disulfide bond between a structurally conserved surface exposed VL Cys and a L Cys; or
c) a first disulfide bond between a structurally conserved surface exposed VH Cys and a first L Cys and a second disulfide bond between a structurally conserved surface exposed VL Cys and a second L Cys; and
wherein the Fab binds to a first antigen on the target cell and the scFv binds to a second antigen on the cell.

D2. The method of embodiment D1, wherein
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position and the L comprises a first L Cys and a second L Cys, wherein the VH Cys and the first L Cys are capable of forming a disulfide bond, and the VL Cys and the second L Cys are capable of forming a disulfide bond.

D3. The method of embodiment D1 or D2, wherein the distance between the VH Cys and the VL Cys is from about 7 Å to about 9 Å or from about 7 Å to about 9 Å.

D4. The method of any one of embodiments D1-D3, wherein the VH Cys is at H3, H5, H40, H43, H46 or H105, wherein the residue numbering is according to Chothia.

D5. The method of any one of embodiments D1-D4, wherein the VL Cys is at L3, L5, L39, L42, L43, L45, L100 or L102, wherein the residue numbering is according to Chothia.

D6. The method of any one of embodiments D1-D5, wherein
a) the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
jj) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102,
qq) the VH Cys is at H105 and the VL Cys is at L43,
   wherein residue numbering is according to Chothia.

D7. The method of any one of embodiments D1-D6, wherein the L comprises a contiguous amino acid sequence derived from an immunoglobulin (Ig) hinge region.

D8. The method of any one of embodiments D1-D7, wherein the Ig hinge region is derived from a human Ig hinge region or a non-human Ig hinge region.

D9. The method of any one of embodiments D1-D8, wherein the Ig hinge region is derived from a human Ig hinge region.

D10. The method of any one of embodiments D1-D9, wherein the human Ig hinge region is an IgG1, IgG2, IgG3, or IgG4 isotype.

D11. The method of any one of embodiments D1-D10, wherein the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

D12. The method of embodiment D11, wherein the L comprises an amino acid sequence C(X)_{y}C (SEQ ID NO: 24), wherein X is Gly, Ser or Pro, and y is an integer from 1 to 3.

D13. The method of any one of embodiments D1-D12, wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

D14. The method of any one of embodiments D1-D13, wherein the L comprises from about 14 to about 19 amino acids, such as about 14, about 15, about 16, about 17, about 18 or about 19 amino acids; and/or the L has a length of from about 14 to about 19 amino acids, such as about 14, about 15, about 16, about 17, about 18 or about 19 amino acids.

D15. The method of any one of embodiments D1-D14 wherein the L comprises the amino acid sequence (X)ₘC(X)_{y}C(X)ₙ(SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

D16. The method of embodiment D15, wherein the L comprises the amino acid sequence (X)ₘC(X)_{y}C(X)ₙ(SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

D17. The method of embodiment D16, wherein the L comprises the amino acid sequence (X)ₘC(X)_{y}C(X)ₙ(SEQ ID NO: 27); wherein X is Gly or Pro, m is an integer from 6 to 9, y is an integer from 1 to 3 and n is an integer from 4 to 6.

D18. The method of any one of embodiments D1-D17, wherein the L comprises the amino acid sequence of SEQ ID NO: 2, SEQ ID NO: 3, SEQ ID NO: 4, SEQ ID NO: 5, SEQ ID NO: 6, or SEQ ID NO: 7.

D19. The method of any one of embodiments D1-D18, wherein the scFv is in the VL-L-VH orientation.

D20. The method of any one of embodiments D1-D18, wherein the scFv is in the VH-L-VL orientation.

D21. The method of any one of embodiments D1-D19, wherein
a) the VH comprises a Cys at H105;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D22. The method of any one of embodiments D1-D19, wherein
a) the VH comprises a Cys at H105;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D23. The method of any one of embodiments D1-D19, wherein
a) the VH comprises a Cys at H105;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D24. The method of any one of embodiments D1-D19, wherein
a) the VH comprises a Cys at H5;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D25. The method of any one of embodiments D1-D19, wherein
a) the VH comprises a Cys at H5;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D26. The method of embodiment D1, wherein
a) the VH comprises a Cys at H5;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D27. The method of any one of embodiments D1-D19, wherein
a) the VH comprises a Cys at H3;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D28. The method of any one of embodiments D1-D19, wherein
a) the VH comprises a Cys at H3;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D29. The method of any one of embodiments D1-D19, wherein
a) the VH comprises a Cys at H3;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D30. The method of any one of embodiments D1-D18 and D20, wherein
a) the VH comprises a Cys at H43;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D31. The method of any one of embodiments D1-D18 and D20, wherein
a) the VH comprises a Cys at H43;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D32. The method of any one of embodiments D1-D18 and D20, wherein
a) the VH comprises a Cys at H43;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D33. The method of any one of embodiments D1-D18 and D20, wherein
a) the VH comprises a Cys at H43;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D34. The method of any one of embodiments D1-D18 and D20, wherein
a) the VH comprises a Cys at H40;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D35. The method of any one of embodiments D1-D18 and D20, wherein
a) the VH comprises a Cys at H40;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D36. The method of any one of embodiments D1-D18 and D20, wherein
a) the VH comprises a Cys at H40;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D37. The method of any one of embodiments D1-D18 and D20, wherein
a) the VH comprises a Cys at H40;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D38. The method of any one of embodiments D1-D18 and D20, wherein
a) the VH comprises a Cys at H46;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D39. The method of any one of embodiments D1-D18 and D20, wherein
a) the VH comprises a Cys at H46;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D40. The method of any one of embodiments D1-D18 and D20, wherein
a) the VH comprises a Cys at H46;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D41. The method of any one of embodiments D1-D18 and D20, wherein
a) the VH comprises a Cys at H46;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

D42. The method of any one of embodiments D21-D41, wherein the L comprises the amino acid sequence of SEQ ID NO: 3.

D43. The method of any one of embodiments D21-D41, wherein the L comprises the amino acid sequence of SEQ ID NO: 6.

D44. The method of any one of embodiments D21-D41, wherein the L comprises the amino acid sequence of SEQ ID NO: 7.

D45. The method of any one of embodiments D1-D44, wherein the binding molecules comprises a heavy chain, a light chain and a polypeptide,
wherein the N-terminus of the heavy chain and the light chain form the Fab;
wherein the polypeptide comprises the scFv at the N-terminus; and
wherein the C-terminus of the polypeptide and the C-terminus of the heavy chain form the Fc region.

D46. The method of any one of embodiments D1 to D45, wherein the Fab binds to a tumor antigen and the scFv binds to a T cell antigen; and wherein optionally the tumor antigen is BCMA and the T cell antigen is CD3.

D47. The method of embodiment D46, wherein the scFv comprises the amino acid sequence of SEQ ID NO: 126 or SEQ ID NO: 128.

D48. The method of embodiment D46 or embodiment D47, wherein (i) the Fab comprises a VH comprising the amino acid sequence of SEQ ID NO: 132, and a VL comprising the amino acid sequence of SEQ ID NO: 129; or (ii) the Fab comprises a VH comprising the amino acid sequence of SEQ ID NO: 137, and a VL comprising the amino acid sequence of SEQ ID NO: 135.

D49. The method of embodiment D47 or embodiment D48, wherein
a) the VH comprises a Cys at H105;
b) the VL comprises a Cys at L43;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

D50. The method of any one of embodiments D1 to D49, wherein the target cell is a tumor cell, thereby eliminating the tumor cell.

D51. The method of any one of embodiments D1 to D50, wherein the method is for treating a disease or disorder in a subject.

D52. The method of embodiment D51, wherein the disease or disorder is a tumor, optionally wherein the disease or disorder is cancer.

D53. The method of embodiment D51, wherein the subject is a human subject.

D54. The method of any one of embodiments D1-D53, wherein the cell is an immune cell.

D54. The method of any one of embodiments D1-D53, wherein the cell is a T cell.

In one set of embodiments, provided are:

E1. A molecule comprising an antigen-binding fragment (Fab) that binds to a first antigen, and a single chain variable fragment (scFv) that binds to a second antigen, and a fragment crystallizable region (Fc region), wherein the scFv comprises a means for stabilizing the scFv.

E2. The molecule of embodiment E1, wherein the scFv comprises a heavy chain variable region (VH), a linker (L) and a light chain variable region (VL), and wherein the means for stabilizing the scFv comprises:
a) a disulfide bond between a structurally conserved surface exposed VH cysteine (Cys) and a L Cys;
b) a disulfide bond between a structurally conserved surface exposed VL Cys and a L Cys; or
c) a first disulfide bond between a structurally conserved surface exposed VH Cys and a first L Cys and a second disulfide bond between a structurally conserved surface exposed VL Cys and a second L Cys.

E3. The molecule of embodiment E2, wherein
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position and the L comprises a first L Cys and the second L Cys, wherein the VH Cys and the first L Cys are capable of forming a disulfide bond, and the VL Cys and the second L Cys are capable of forming a disulfide bond.

E4. The molecule of embodiment E2 or E3, wherein the distance between the VH Cys and the VL Cys is from about 5 Å to about 10 Å or from about 7 Å to about 9 Å.

E5. The molecule of any one of embodiments E1-E3, wherein the VH Cys is at H3, H5, H40, H43, H46 or H105, and/or wherein the VL Cys is at L3, L5, L39, L42, L43, L45, L100 or L102, and wherein the residue numbering is according to Chothia.

E6. The molecule of any one of embodiments E1-E5, wherein
a) the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
jj) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102, qq) the VH Cys is at H105 and the VL Cys is at L43,
   wherein the residue numbering is according to Chothia.

E7. The molecule of any one of embodiments E1-E6, wherein the L comprises a contiguous amino acid sequence derived from an immunoglobulin (Ig) hinge region.

E8. The molecule of any one of embodiments E1-E7, wherein the Ig hinge region is derived from a human Ig hinge region or a non-human Ig hinge region.

E9. The molecule of any one of embodiments E1-E8, wherein the Ig hinge region is derived from a human Ig hinge region.

E10. The molecule of any one of embodiments E1-E9, wherein the human Ig hinge region is an IgG1, IgG2, IgG3, or IgG4 isotype.

E11. The molecule of any one of embodiments E1-E10, wherein the L comprises an amino acid sequence C(X)_{y}C (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3.

E12. The molecule of embodiment E11, wherein the L comprises an amino acid sequence C(X)_{y}C (SEQ ID NO: 24), wherein X is Gly, Ser or Pro, and y is an integer from 1 to 3.

E13. The molecule of any one of embodiments E1-E12, wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52).

E14. The molecule of any one of embodiments E1-E13, wherein the L comprises from about 14 to about 19 amino acids, such as about 14, about 15, about 16, about 17, about 18 or about 19 amino acids.

E15. The molecule of any one of embodiments E1-E14 wherein the L comprises the amino acid sequence (X)ₘC(X)_{y}C(X)ₙ(SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6.

E16. The molecule of embodiment E15, wherein the L comprises the amino acid sequence (X)ₘC(X)_{y}C(X)ₙ(SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6.

E17. The molecule of embodiment E16, wherein the L comprises the amino acid sequence (X)ₘC(X)_{y}C(X)ₙ(SEQ ID NO: 27); wherein X is Gly or Pro, m is an integer from 6 to 9, y is an integer from 1 to 3 and n is an integer from 4 to 6.

E18. The molecule of any one of embodiments E1-E17, wherein the L comprises the amino acid sequence of SEQ ID NO: 2, SEQ ID NO: 3, SEQ ID NO: 4, SEQ ID NO: 5, SEQ ID NO: 6, or SEQ ID NO: 7.

E19. The molecule of any one of embodiments E1-E18, wherein the scFv is in the VL-L-VH orientation.

E20. The molecule of any one of embodiments E1-E18, wherein the scFv is in the VH-L-VL orientation.

E21. The molecule of any one of embodiments E1-E19, wherein
a) the VH comprises a Cys at H105;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E22. The molecule of any one of embodiments E1-E18, wherein
a) the VH comprises a Cys at H105;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E23. The molecule of any one of embodiments E1-E18, wherein
a) the VH comprises a Cys at H105;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E24. The molecule of any one of embodiments E1-E18, wherein
a) the VH comprises a Cys at H5;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E25. The molecule of any one of embodiments E1-E18, wherein
a) the VH comprises a Cys at H5;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E26. The molecule of any one of embodiments E1-E18, wherein
a) the VH comprises a Cys at H5;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E27. The molecule of any one of embodiments E1-E18, wherein
a) the VH comprises a Cys at H3;
b) the VL comprises a Cys at L42;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E28. The molecule of any one of embodiments E1-E18, wherein
a) the VH comprises a Cys at H3;
b) the VL comprises a Cys at L45;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E29. The molecule of any one of embodiments E1-E18, wherein
a) the VH comprises a Cys at H3;
b) the VL comprises a Cys at L39;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E30. The molecule of any one of embodiments E1-E18 and E20, wherein
a) the VH comprises a Cys at H43;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E31. The molecule of any one of embodiments E1-E18 and E20, wherein
a) the VH comprises a Cys at H43;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E32. The molecule of any one of embodiments E1-E18 and E20, wherein
a) the VH comprises a Cys at H43;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E33. The molecule of any one of embodiments E1-E18 and E20, wherein
a) the VH comprises a Cys at H43;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E34. The molecule of any one of embodiments E1-E18 and E20, wherein
a) the VH comprises a Cys at H40;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E35. The molecule of any one of embodiments E1-E18 and E20, wherein
a) the VH comprises a Cys at H40;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E36. The molecule of any one of embodiments E1-E18 and E20, wherein
a) the VH comprises a Cys at H40;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E37. The molecule of any one of embodiments E1-E18 and E20, wherein
a) the VH comprises a Cys at H40;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E38. The molecule of any one of embodiments E1-E18 and E20, wherein
a) the VH comprises a Cys at H46;
b) the VL comprises a Cys at L100;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E39. The molecule of any one of embodiments E1-E18 and E20, wherein
a) the VH comprises a Cys at H46;
b) the VL comprises a Cys at L102;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E40. The molecule of any one of embodiments E1-E18 and E20, wherein
a) the VH comprises a Cys at H46;
b) the VL comprises a Cys at L5;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E41. The molecule of any one of embodiments E1-E18 and E20, wherein
a) the VH comprises a Cys at H46;
b) the VL comprises a Cys at L3;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VH-L-VL orientation.

E42. The molecule of any one of embodiments E21-E41, wherein the L comprises the amino acid sequence of SEQ ID NO: 3.

E43. The molecule of any one of embodiments E21-E41, wherein the L comprises the amino acid sequence of SEQ ID NO: 6.

E44. The molecule of any one of embodiments E21-E41, wherein the L comprises the amino acid sequence of SEQ ID NO: 7.

E45. The molecule of any one of embodiments E1-E44, wherein the binding molecules comprises a heavy chain, a light chain and a polypeptide,
wherein the N-terminus of the heavy chain and the light chain form the Fab;
wherein the polypeptide comprises the scFv at the N-terminus; and
wherein the C-terminus of the polypeptide and the C-terminus of the heavy chain form the Fc region.

E46. The molecule of any one of embodiments E1 to E45, wherein the Fab binds to a tumor antigen and the scFv binds to a T cell antigen; and wherein optionally the tumor antigen is BCMA and the T cell antigen is CD3.

E47. The molecule of embodiment E46, wherein the scFv comprises the amino acid sequence of SEQ ID NO: 126 or SEQ ID NO: 128.

E48. The molecule of embodiment E46 or embodiment E47, wherein the Fab comprises a VH comprising the amino acid sequence of SEQ ID NO: 132, and a VL comprising the amino acid sequence of SEQ ID NO: 129; or (ii) wherein the Fab comprises a VH comprising the amino acid sequence of SEQ ID NO: 137, and a VL comprising the amino acid sequence of SEQ ID NO: 135.

E49. The method of embodiment E47 or embodiment E48, wherein
a) the VH comprises a Cys at H105;
b) the VL comprises a Cys at L43;
c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and
d) the scFv is in the VL-L-VH orientation.

E50. A means for producing the molecule of any one of embodiments E1-E49.

E51. A method for directing or engaging a cell to a target cell comprising contacting the target cell with the molecule of any one of embodiments E1-E49.

E52. A method for eliminating or inhibiting a target cell comprising contacting the target cell with the molecule of any one of embodiments E1-E49.

E53. A method for treating a disease or disorder in a subject comprising administering to the subject the molecule of any one of embodiments E1-E49.

In one set of embodiments, provided are:

F1. The molecule of any one of embodiments A1-A55 for use in a medicament.

F2. The molecule of any one of embodiments A1-A55 for use in treating a disease or disorder.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 6. EXAMPLES

### 6.1 Example 1: CD3/CRIS7A and CD3/CRIS7B scFv and spFv Stability

6.1.1 Expression and Purification of Anti-CD3 Cris7a and Cris7b scFv/spFv

All scFv and spFv molecules were cloned into a CMV promoter driven mammalian expression vector. These constructs were transfected into Expi293 cells using manufacturer protocols and cells were cultured for 5 days. Each Protein was purified from the clarified supernatant on 1 ml His-TRAP HP columns (GE Healthcare) via an AktaXpress system (GE Healthcare). The column was prepared with a gradient of 0-100% Elution Buffer (Wash Buffer: 50 mM Tris, pH 7.5, 500 mM NaCl, 20 mM Imidazole; Elution Buffer: 50 mM Tris, pH 7.5, 500 mM NaCl, 500 mM Imidazole) to remove loosely bound nickel and then re-equilibration in DPBS. The cleared supernatant was first adjusted to 50 mM Tris, pH 7.5 and 20 mM imidazole and then loaded over 1 mL HisTRAP HP column @4° C. 0.8 mL/min. The column was then washed with PBS until stable baseline was obtained. Then the column was further washed with 20 CV of Wash Buffer, eluted with Elution buffer into a single injection loop and desalted in 1×DPBS over 26/10 HiPrep Desalting Column and fractions collected. Fractions containing the purified protein were then pooled and concentrated. The scFv and spFv proteins were dialyzed into DPBS for thermal stability measurements.

scFv/spFv Stability by Differential Scanning Calorimetry (DSC) Conformational stability of the Cris7a or Cris7b scFvs and their stapled spFvs were measured by differential scanning calorimetry (DSC) using a Microcal Capillary DSC instrument (Malvern Instruments) with an autosampler. Samples with the matching buffer were scanned at a rate of 60° C./hr in the range of 25˜100° C. with no feedback option. Six buffer-buffer only scans were performed before protein samples to establish thermal history and stable baseline. Raw DSC data were subjected to buffer blank subtraction, normalized by their protein concentration and baseline subtraction. Processed data were fitted using non-2 state transition model using Origin 7 software (version 7.0552). Iterative curve fitting was performed to derive thermodynamic parameters associated with the melting, e.g. thermal stability, enthalpy.

### 6.1.2 Surface Plasmon Resonance

Binding of the Cris7b scFvs and the stapled spFv to recombinant CD3 antigen were measured by surface plasmon resonance using a Biacore 8K instrument (Cytiva, formally GE Healthcare) at 25° C. Goat anti-human Fcγ protein (Jackson ImmunoResearch 109-005-098), was directly immobilized on a CM4 chip (Series S CM4 Sensor chip, Cat #BR100534) using standard amine coupling. Final ˜4000 Rus were immobilized on each channel. Samples with Cris7b scFv or spFv containing bi-specifics were captured by the anti-human Fcγ surface with levels ranging 100-250 Rus, followed by the binding of a series of 5 antigen concentrations of Human CD3E-CD3D Heterodimer Protein (Acro Cat #CDDH52W1) starting at 300 nM in 3 fold dilution (300 nM˜3.7 nM) using single cycle kinetics method. Association and dissociation times were 150 s and 600 s, respectively. The surface was regenerated using 0.85% phosphoric acid with three short pulses, 20 s each at 50 μl/min flow rate to remove the captured/bound antibody/antigen complexes before the next round of interaction. Running buffer was 0.01 M HEPES pH 7.4, 0.15 M NaCl, 0.05% v/v Surfactant P20. Raw binding data were processed by double referencing via subtracting 1) signals from the antigen binding to the empty chip surface (FC1 on each channel) and 2) signals from the proceeding buffer blank injection. Processed data were then subject to a 1:1 simple Langmuir binding model analysis to derive the kinetic (ka, kd) and affinity (KD) parameters using the Biacore Insight Evaluation software version 2 (Cytiva).

6.1.3 Comparison of Stability of Anti-CD3 Cris7a and Cris7b scFv/spFv

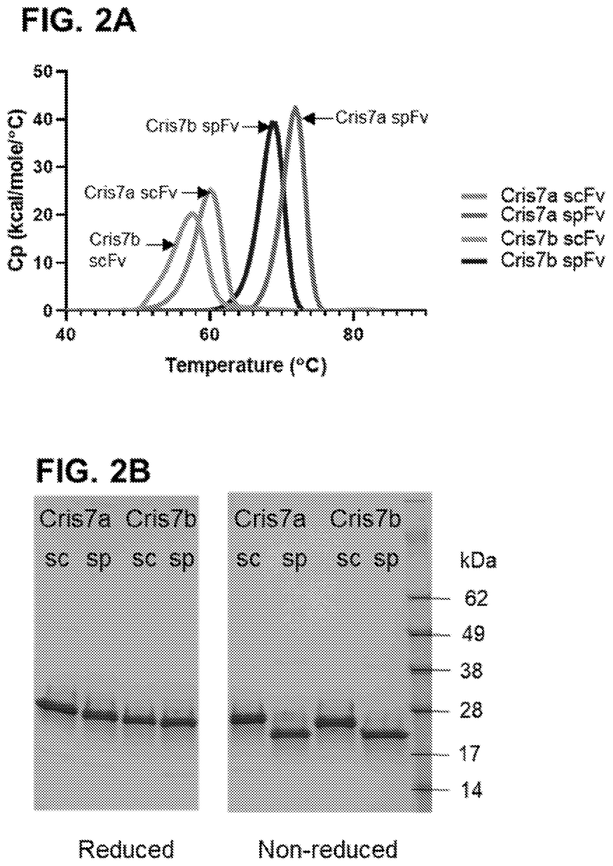

Cris7a and Cris7b were derived from anti-CD3 variants of CRIS7 that had potential for T cell redirecting. The Tm of their scFv moieties had less than ideal thermal stability (FIG. 2A) with Tm at 59.7 and 57.1° C., respectively. See Table 6.

TABLE 6
Tm of Cris7a and Cris7b scFvs and spFvs

| | Non-2 state model analyses | | | |
|---|---|---|---|---|
| | Tm | Tm | ΔH | ΔH Error |
| Protein ID | (° C.) | Error (° C.) | (kcal/mol) | (kcal/mol) |
| Cris7a scFv | 59.7 | 0.04 | 1.17E+05 | 1.99E+03 |
| Cris7a spFv (staple) | 71.6 | 0.02 | 1.80E+05 | 2.25E+03 |
| Cris7b scFv | 57.1 | 0.05 | 1.19E+05 | 2.01E+03 |
| Cris7b spFv (staple) | 68.6 | 0.03 | 1.76E+05 | 3.09E+03 |

Stapling was applied to the scFv constructs in FIG. 2A in the VL-linker-VH format. The SDS-PAGE of these scFv molecules showed that the non-reduced spFvs migrated faster than the corresponding scFvs, whereas the reduced proteins migrated identically. See FIG. 2B. These data indicated that the stapling disulfide bonds were formed, which resulted in more compact structures of the spFvs. In comparison with the corresponding scFvs, the Tms of the spFv were higher by more than 11° C. from scFv to spFv (FIG. 2A and Table 6). Stapled scFvs (spFv) also displayed ˜50% more enthalpy than those of the parental unstapled scFvs (Table 6). The increase in melting enthalpy indicated stronger VH/VL interactions, resulting from stronger VL/VH interactions and/or restraints on VL/VH relative movements due to stapling.

### 6.2 Example 2: CD3/BCMA Bi-Specific Constructs, Properties and Activity

### 6.2.1 Expression and Purification of CD3/BCMA Bi-Specifics.

To further study the activity and properties of the stapled scFv molecules several proof-of-concept bi-specific constructs, in which anti-CD3 scFv/spFv were paired with anti-BCMA Fab using the Knob-in-Hole Fc heterodimerization platform, were generated. The anti-BCMA arm, BCMB749, was a mouse monoclonal antibody (provided by Xie-fan Lin-Schmidt). BCMB749h was a humanized variant in which BCMB749 CDRs (AbM definition) were grafted onto a human VH and VL with several back mutations (1 VH and VL acceptors were selected). BCMB749 and BCMB749h Fabs were then paired with two different anti-CD3 (Cris7b and CD3B219) scFv and spFv to generate BCMA targeting bi-specific molecules. The various constructs are given in Table 7.

TABLE 7
The CD3/BCMA bi-specific molecules generated for this study.

| Name | Description | Light Chain | Heavy Chain 1 | Heavy Chain 2 |
|---|---|---|---|---|
| BCMB1056 | BCMB749 × Cris7b scFv | BCMB749 LC | BCMB749 HC1 | Cris7b VL-VH scFv HC2 |
| BCMB1052 | BCMB749 × Cris7b spFv | BCMB749 LC | BCMB749 HC1 | Cris7b VL-VH spFv HC2 |
| BCMB1055 | BCMB749h × Cris7b scFv | BCMB749h LC | BCMB749h HC1 | Cris7b VL-VH scFv HC2 |
| BCMB1050 | BCMB749h × Cris7b spFv | BCMB749h LC | BCMB749h HC1 | Cris7b VL-VH spFv HC2 |
| BCMB1051 | BCMB749 × CD3B219 spfv | BCMB749 LC | BCMB749 HC1 | CD3B219a99v spFv HC2 |
| BCMB1049 | BCMB749h × CD3B219 spFv | BCMB749h LC | BCMB749h HC1 | CD3B219a99v spFv HC2 |
| BCMB1054 | BCMB749 × CD3B219 scfv | BCMB749 LC | BCMB749 HC1 | CD3B219a99v scFv HC2 |
| BCMB1053 | BCMB749h × CD3B219 scFv | BCMB749h LC | BCMB749h HC1 | CD3B219a99v scFv HC2 |

All bispecific scFv and spFv molecules, along with corresponding Fab HC and LC arms, were cloned into a CMV promoter driven mammalian expression vector. These constructs were transfection into ExpiCHO cells using manufacturer protocols and cells were cultured for 7 days. All samples were harvested and resulting supernatant was filtered through a 0.2 μm filter for clarification. Samples were purified through a two-step process. Clarified supernatants were first passed over a HITRAP MABSELECT SURE (Protein A) 1 mL column (GE Healthcare). The column was equilibrated and washed in 1×DPBS; final samples were eluted in 0.1 M sodium acetate pH 3.5. Once the protein was eluted, samples were immediately neutralized with 2.5M Tris-HCl pH 7.5, and then loaded over 0.5 mL CaptureSelect CH1-XL Affinity Matrix (ThermoFisher). CH1 column was equilibrated and washed in 1×DPBS, and final samples were eluted in 0.1 M sodium acetate pH 3.5. Samples were dialyzed into 1×DPBS for storage, and 30 μL of sample was assessed for sample quality and purity on Agilent ADVANCEBIO SEC 300A (Agilent) using 1×DPBS as running buffer. Final samples were assessed to determine concentration by A280 and stored at 4° C.

### 6.2.2 Tm/Tagg by Differential Scanning Fluorimetry (NanoDSF).

Conformational stability of the bi-specific molecules was assessed using advanced differential scanning fluorimetry (nanoDSF) technology, by monitoring the intrinsic fluorescence of tryptophan upon thermal unfolding. The unfolding was measured by loading each sample into 24 well capillary (NanoTemper, Cat #PR-AC002) from a 384 well sample plate (ThermoNunc, Cat #264573), with a heating ramp of 1° C./minute between 20˜95° C. using the Prometheus NT.48 instrument (NanoTemper Technologies GmbH). Each sample was measured at 1 mg/ml in phosphate buffer saline (PBS) in duplicates. The intrinsic fluorescence of each sample at 330 and 350 nm was used to monitor unfolding during temperature ramp and recorded as changes in fluorescence intensity over time. Thermal melting data were processed using the PR.STABILITYANALYSIS v1.0.2 software. The processed data contains integrated data and first derivation analysis for 330 nm, 350 nm, Ratio 330/350, and scatter data for all the samples. Final analysis results with the annotated transition data for each sample were exported in excel table format.

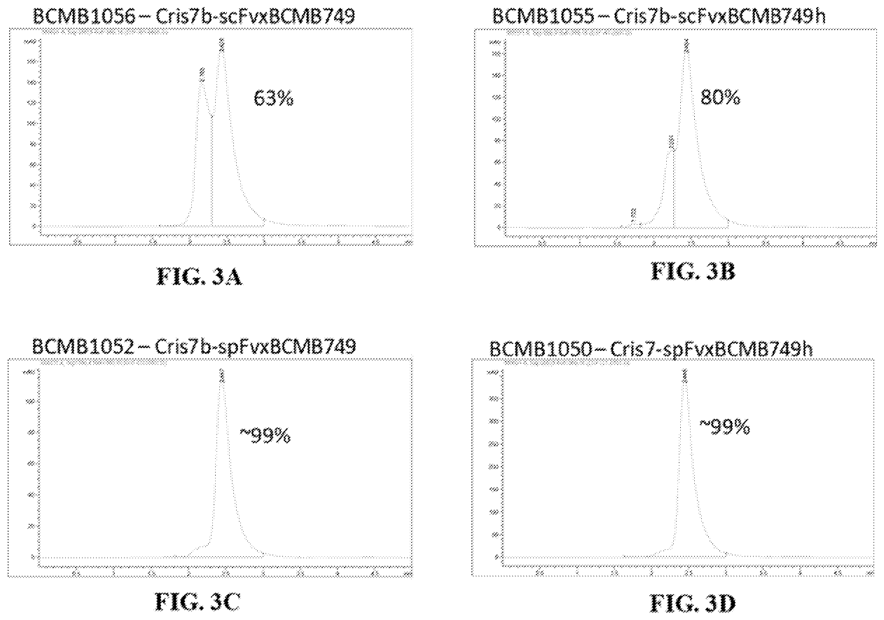

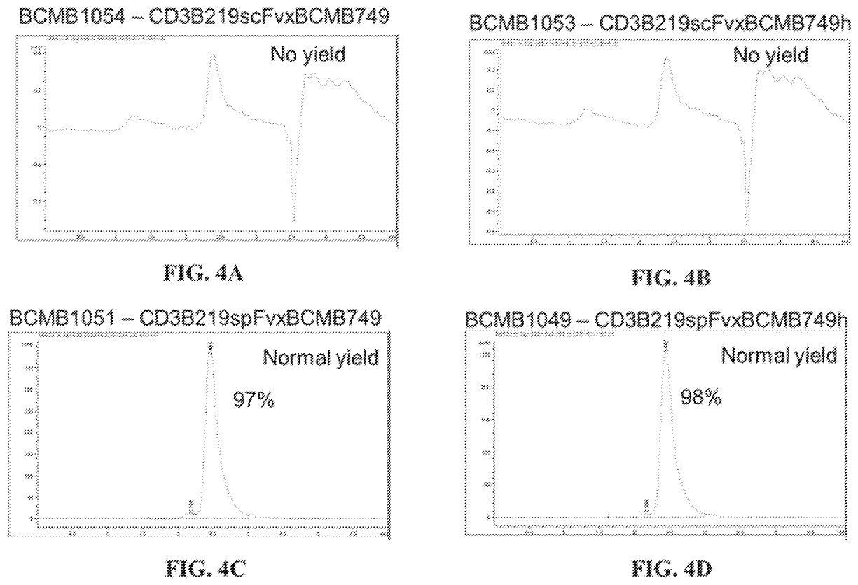

For the Cris7b containing bi-specifics, the spFv containing molecules B1052 and B1050 with murine and humanized Fab arm, respectively, both had 99% or higher bi-specific monomer, whereas their scFv containing counterparts, B1056 and B1055, had 60-80% bi-specific monomer and 20-40% dimer based on SEC results (FIGS. 3A-3D). For the bi-specific constructs with CD3B219, there was low expression and purification yields after purification for the scFv containing constructs B1054 and B1053. This result was true on multiple repeats. It could be a result of expression or DNA sequences for the scFv. On the other hand, the spFv containing constructs, B1051 and B1049, gave high quality and yield final bi-specific monomer proteins (FIGS. 4A-4D). These data indicated that stapling in both CD3 scFv arms (Cris7b and CD3B219) significantly improved the bi-specific product yields and quality.

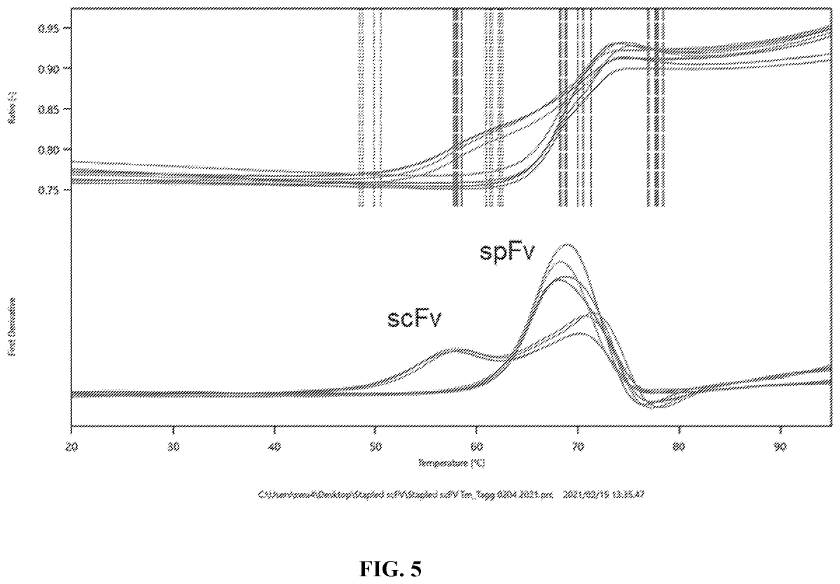

Next, the impact of stapling on the thermal stability on the bi-specific constructs was evaluated by NanoDSF (FIG. 5 and Table 8). For Cris7b, the scFv containing constructs had a melting transition at a Tm of ˜58.0° C. (B1056 and B1055) with Tonset at 48.0-50.0° C. For the corresponding spFv contain bi-specifics (B1052 and B1050), this low Tm transition disappeared and the first transition had a Tm of ˜68.5° C. Moreover, the Tonset of these two spFv bi-specific proteins was at ˜61° C. Both of these were interpreted to indicate approximately 10° C. or higher stability improvement for the spFv moiety. For the CD3B219 spFv constructs, since there was no scFv proteins for comparison, it was not possible to measure the difference. On the other hand, both of them had high Tm (˜69° C.) and Tonset (˜62° C.).

TABLE 8
Thermal stability of CD3/BCMA bi-specific molecules

| Protein Batch | BCMA | | | | | | |
|---|---|---|---|---|---|---|---|
| Name | Fab | CD3 arm | Tonset | Tm1 | Tm2 | Tm3 | Tagg |
| BCMB1056.001 | BCMB749, | scfv cris7b | 48.6 | 57.9 | 70.0 | 77.8 | 70.0 |
| | murine | | | | | | |
| BCMB1052.001 | BCMB749, | stapled | 60.9 | 68.3 | | | 70.3 |
| | murine | cris7b | | | | | |
| BCMB1055.001 | BCMB749, | scfv cris7b | 50.2 | 58.3 | 70.9 | 78.1 | 70.5 |
| | humanized | | | | | | |
| BCMB1050.001 | BCMB749, | stapled | 61.4 | 68.9 | 76.9 | | 69.8 |
| | humanized | cris7b | | | | | |
| BCMB1051.001 | BCMB749, | stapled | 62.3 | 68.3 | | | 70.2 |
| | murine | CD3B219 | | | | | |
| BCMB1049.001 | BCMB749, | stapled | 62.4 | 68.8 | 77.9 | | 70.4 |
| | humanized | CD3B219 | | | | | |

Stapling did not alter anti-CD3 binding to CD3 antigens by SPR measurements. The scFv and corresponding spFv proteins had very similar binding affinity for CD3. See Table 9.

TABLE 9
Surface Plasmon Resonance (SPR) measurement of CD3 binding.

| | Sample Info. | | BCMA binding (BCMW37.DB.001) | | | CD3W220.001 binding |
|---|---|---|---|---|---|---|
| Protein Batch | BCMA | CD3 | ka | | | ka |
| Name | Fab | arm | (1/Ms) | kd (1/s) | KD (M) | (1/Ms) |
| BCMB1056.001 | BCMB749, | scfv | 1.49E+07 | 3.89E−02 | 2.61E−09 | 2.49E+05 |
| | murine | cris7b | | | | |
| BCMB1052.001 | BCMB749, | stapled | 1.42E+07 | 3.90E−02 | 2.74E−09 | 2.10E+05 |
| | murine | cris7b | | | | |
| BCMB1055.001 | BCMB749, | scfv | 1.26E+07 | 2.23E−02 | 1.77E−09 | 2.67E+05 |
| | humanized | cris7b | | | | |
| BCMB1050.001 | BCMB749, | stapled | 1.20E+07 | 2.10E−02 | 1.75E−09 | 1.84E+05 |
| | humanized | cris 7b | | | | |
| BCMB1051.001 | BCMB749, | stapled | 1.67E+07 | 4.13E−02 | 2.47E−09 | 7.95E+04 |
| | murine | CD3B219 | | | | |
| BCMB1049.001 | BCMB749, | stapled | 1.38E+07 | 2.19E−02 | 1.59E−09 | 8.69E+04 |
| | humanized | CD3B219 | | | | |

| | | | CD3EG (Acro) binding | | |
|---|---|---|---|---|---|
| Protein Batch | CD3W220.001 binding | | ka | | |
| Name | kd (1/s) | KD (M) | (1/Ms) | kd (1/s) | KD (M) |
| BCMB1056.001 | 2.37E−01 | 9.51E−07 | 5.20E+05 | 3.56E−01 | 6.83E−07 |
| BCMB1052.001 | 1.05E−01 | 5.02E−07 | 8.18E+05 | 2.15E−01 | 2.63E−07 |
| BCMB1055.001 | 1.99E−01 | 7.46E−07 | 6.89E+05 | 4.40E−01 | 6.39E−07 |
| BCMB1050.001 | 1.21E−01 | 6.57E−07 | 4.53E+05 | 2.17E−01 | 4.79E−07 |
| BCMB1051.001 | 1.71E−03 | 2.15E−08 | 5.69E+05 | 1.49E−03 | 2.61E−09 |
| BCMB1049.001 | 2.05E−03 | 2.36E−08 | 4.41E+05 | 1.96E−03 | 4.45E−09 |

### 6.2.3 BCMA/CD3 Cytotoxicity Assay.

To determine the effect of BCMA targeting test molecules upon T cell activation and killing potential of tumor cells, the INTELLICYT IQUE3 and FORECYT software (Sartorius) was utilized. H929-Fluc-GFP cells served as target cells for two human donor Pan T cells (Hemacare). The assay was set up in a 96-well plate at a T cell to target ratio of 3:1. Test molecules were added at a starting concentration of 10 nM and serially diluted at 1:4 in complete media. All molecules were tested in duplicate at minimum.

Detection of killing and T cell activation status was assessed 72 h later by flow cytometry. Endogenous GFP expressed in H929 was used to separate T cells from target cells. Cytotoxicity was measured using Near-IR Live/Dead stain (ThermoFisher, CatL34976), while activation in CD4 and CD8 T cells was assessed with anti-human CD25-BV650 (BD Biosciences), anti-human CD4-BV510 (Biolegend) and anti-human CD8-PE/Cy7 (Biolegend). The cytotoxicity and CD25 MFI data were exported and analyzed with PRISM (GRAPHPAD). The data were log-transformed, and 4 parameter logistically fit to generate regression curves for reporting of EC50.

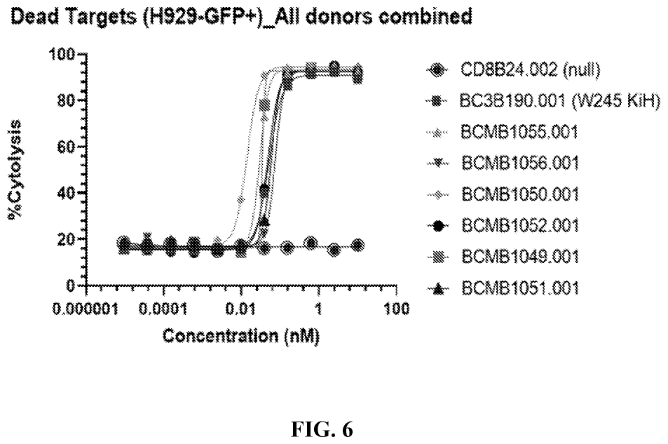

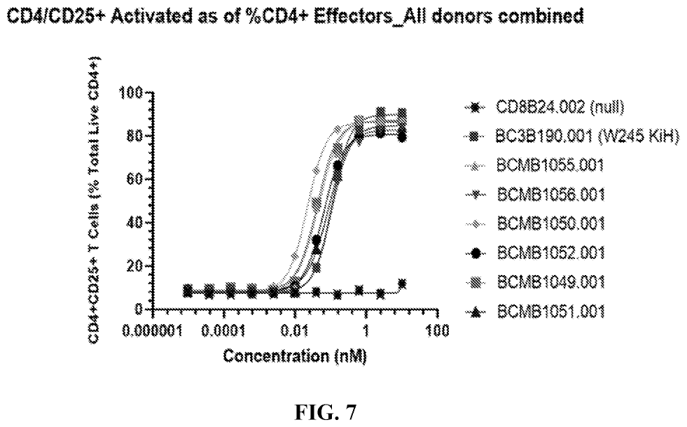

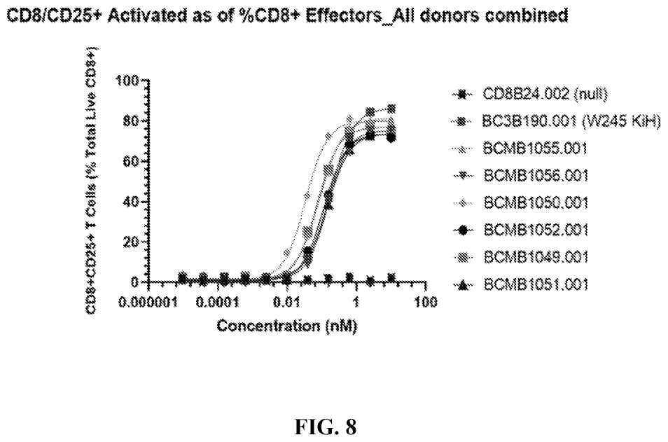

All bi-specific proteins potently killed BCMA⁺H929-GFP⁺ cells in a cytotoxicity assay with very similar EC50 (FIG. 6), whereas a negative control bi-specific with a Cris7b scFv/non-targeting Fab (CD8B24) showed no killing activity. B1050, which contains the Cris7b spFv and the humanized B647, appeared to have the highest killing activity among these CD3-redirecting molecules. These constructs also activated CD4⁺ and CD8⁺ T cells with similar EC50s (FIGS. 7 and 8) whereas the negative control molecule did not activate T cells, indicating the H929 cell killing was the result of T cell activation by CD3 redirecting. All EC50 values are shown in Table 10.

TABLE 10
EC50 values of CD3/BCMA bispecific molecule cytotoxicity assay.

| | CD8B24 | BC3B190 | | | | | | |
|---|---|---|---|---|---|---|---|---|
| | (null) | (W245 KiH) | BCMB1055 | BCMB1056 | BCMB1050 | BCMB1052 | BCMB1049 | BCMB1051 |
| EC50/Cmax % Cytolysis | | | | | | | | |
| Bottom | 16.68 | 15.97 | 16.06 | 17.17 | 17 | 15.44 | 16.46 | 16.68 |
| Top | no fit | 91 | 93.19 | 92.74 | 94.58 | 93.38 | 92.71 | 92.42 |
| EC50 (nM) | no fit | 0.05224 | 0.03013 | 0.07582 | 0.01413 | 0.04893 | ~0.03485 | 0.05942 |
| EC50/CD4/CD25 + Cmax | | | | | | | | |
| Bottom | 7.583 | 7.521 | 7.455 | 8.294 | 8.053 | 8.118 | 8.584 | 8.666 |
| Top | no fit | 90 | 87.4 | 82.69 | 86.38 | 80.86 | 86.84 | 84.91 |
| EC50 (nM) | no fit | 0.1093 | 0.04474 | 0.1052 | 0.02216 | 0.06274 | 0.04093 | 0.08699 |
| EC50/CD8/CD25 + Cmax | | | | | | | | |
| Bottom | no fit | 1.209 | 0.9695 | 1.417 | 1.317 | 0.9409 | 1.645 | 1.672 |
| Top | no fit | 86.54 | 81.24 | 75.32 | 80.06 | 73.52 | 76.97 | 73.98 |
| EC50 (nM) | no fit | 0.1718 | 0.08588 | 0.1502 | 0.03431 | 0.1169 | 0.07442 | 0.1443 |

### 6.2.4 Large Scale Protein Expression and Purification

Three of the bispecific proteins (TD01B46, TD01B48 and TD01B49) were further expressed at a larger scale expression at (500 ml or 1 L) as described as for small volumes. These proteins were purified to high homogeneity using a slightly different process as follows. Half a liter of clarified cell culture supernatants of select samples were loaded onto pre-equilibrated 5 mL prepacked HITRAP MABSELECT PRISMA columns (Cytiva) with 1×dPBS (pH 7.2) on AKTA PURE System (GE Healthcare). Columns were washed with 5 column volumes (CVs) of 1×dPBS (pH 7.2). Following the wash, 3-5 CVs of 100 mM sodium acetate (pH 3.5) were used to elute the bound antibodies. In line with collection of the enriched fractions post elution, proteins were neutralized with 15% (v/v) 2.5 M Tris-HCl (pH 7.5) using a mixing valve on the Akta Pure system. The collected material was pooled together of each protein separately, and syringe-filtered with 0.2-μm filter. Samples were loaded onto pre-equilibrated 5 mL CAPTURESELECT CH1-XL prepacked affinity columns (Thermo Scientific) on Akta Xpress systems (Amersham Biosciences Corp.) and washed with 4 CVs of 1×dPBS (pH 7.2). The bound antibodies were then eluted off of the columns using 5 CVs of 100 mM sodium acetate (pH 3.5) and the enriched fractions were collected. The collected material was neutralized to final volume of 15% (v/v) 2.5 M Tris-HCl (pH 7.5) and syringe-filtered with 0.2-μm filters. Samples were diluted 8-10-fold with 20 mM MES (pH 5.5) and loaded onto 180 mL CAPTO S IMPACT column (Cytiva) using AKTA PURE System (GE Healthcare) which was equilibrated with 20 mM MES (pH 5.5). The column was washed with 1 CV of 20 mM MES (pH 5.5) and 3 CVs of 20 mM MES (pH 6.5) to remove loosely bound impurities. The proteins were eluted with 15 CVs of buffer B (20 mM MES, 1M sodium chloride, pH 6.5) over a linear gradient (0-30% buffer B). Fractions were pooled and passed through 0.2-μm filters. All samples were loaded onto a pre-equilibrated with 1×dPBS (pH 7.2) 120 mL Superdex 200 pg SEC column (Cytiva) using AKTA AVANT System (GE Healthcare). Proteins were eluted off the column with 1.5 CVs 1×dPBS (pH 7.2). The concentration of purified protein was determined by absorbance at 280 nm with 0.1 and 0.7 mm pathlengths on a DROPSENSE spectrophotometer. The quality of the purified protein was assessed by SDS-PAGE (3.5 μg of sample, Invitrogen, NuPage 4-12% Bis-Tris) and analytical size exclusion HPLC (20 μg sample; column: TSKgel BioAssist G3SW×1, 7.8 mm ID×30 cm H, 5 μm, TOSOH; guard column: TSKgel BioAssist SW×1 guard column, 6 mm ID×4 cm H, 7 μm, TOSOH; Agilent HPLC system) at 1 mL/min for 20 min using 200 mM sodium phosphate (pH 6.8) as the running buffer. The endotoxin level was measured using a turbidometric LAL assay (PYROTELL®-T, Associates of Cape Cod; Falmouth, MA).

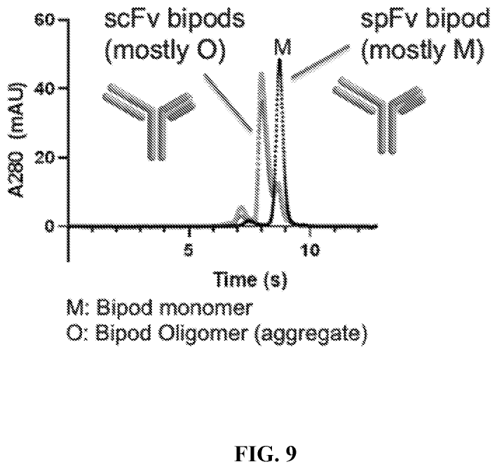

One set of constructs was chosen to move into larger scale expression and purification to generate large batches of highly purified recombinant antibodies that could be used in a panel of analyses to further test the stapling technology. Based on the data as described above, three Cris7b containing molecules were chosen—TD01B46 (BCMB749×Cris7b spFv), TD01B48 (BCMB749×Cris7b scFv G4S) and TD01B49 (BCMB749×Cris7b scFv Bird). Large-scale expression in Expi-CHO and a more thorough purification of these antibody samples revealed more pronounced trends in product quality and yield, as was seen in the small-scale purified samples (FIG. 9). Production of scFv-containing bispecific samples (B48, B49) showed a large, higher molecular weight oligomeric peak (“O”) as dominating the post-CH1 purified sample. In comparison, the spFv-containing bispecific (B46) remained a dominant peak that corresponded to the desired monomeric peak. These data together emphasize the improvement shown in protein production and quality when the stapling technology was implemented.

### 6.2.5 High Concentration and Heat Stress Study

For this study, 10 kDa MWCO Amicon filtration units (Cat #UFC803024) were washed with water at 4200 g for 6 min. TD01B46 (2 mg/ml) and TD01B49 proteins (0.5 mg/ml) were each loaded in a pre-washed filter and centrifuged at 4200 rpm for 15 minutes at a time. Protein concentrations were measured using the SoloVPE instrument (C Technologies, NJ, USA). The final concentrations were 68 mg/ml and 48 mg/ml for the stapled and unstapled bi-specific proteins, respectively. Each protein sample was divided into two equal parts, and one stored at 4° C. and the other stored at 40° C. Every 2 weeks, 50 μl of protein was taken from each incubated sample and diluted to 1 mg/ml with 1×dPBS; 20 μg of the aliquoted sample was loaded onto an analytical size exclusion HPLC (TSKgel BioAssist G3SW×1, 7.8 mm ID×30 cm H, Sum, TOSOH; guard column: TSKGEL BIOASSIST SW×1 guard column, 6 mm ID×4 cm H, 7 um, TOSOH; Agilent HPLC system) and monitored for separation of the sample at UV 280 nm, at 1 mL/min for 20 min using 200 mM sodium phosphate (pH 6.8) as the running buffer.

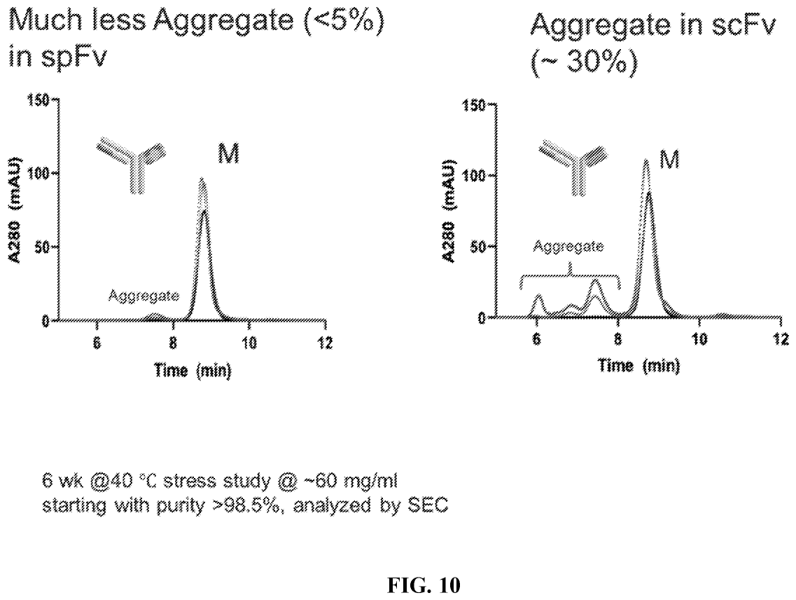

The impact of spFv on aggregation induced by heat stress was further evaluated, as a predictive indicator of protein shelf stability at 4° C. (Bailly et al, (2020); mAbs; Volume 12, Issue 1). The highly purified Cris7b scFv/spFv bi-specifics (TD01B49 and TD01B46, respectively) (purity >98.5%) were concentrated in DPBS to ˜60 mg/ml. The concentrated samples were then incubated at 4 and 40° C. At 2 week intervals, a small aliquot of each sample was diluted to 1 mg/ml and then run on aSEC. The results are shown in FIG. 10. Over a 6 week incubation period at 4° C., the spFv bispecific TD01B46 (left panel) remained a monomer, while at 40° C. it showed a modest increase to about 5% aggregate species at 6 weeks. By contrast, over the same period at either 4° C. or 40° C., the scFv bispecific TD01B49 (right panel) showed a large increase to about 18% and 32% aggregate species, respectively. These data demonstrate that spFv containing bispecifics were much more resistant to heat induced aggregation at high protein concentration.

### 6.2.6 Bio-Layer Interferometry of Cris7 Bispecific Molecules

Binding of the Cris7b scFv and stapled spFv bispecific molecules to recombinant CD3 antigen (human CD3 epsilon and CD3 delta heterodimer protein, Acro Biosystems) and recombinant BCMA antigen were measured by biolayer interferometry (BLI) using an Octet HTX instrument (Sartorius, formerly ForteBio). To evaluate BCMA binding, Streptavidin (SA) capture sensors (Sartorius) were loaded with biotinylated-BCMA protein to ˜1 nm signal in PBS. Bispecific samples were loaded to antigen coated SA sensors at 7 antibody concentrations starting at 100 nM in 2-fold dilution (100 nM˜1.5 nM), diluted in 1×DPBS with 0.05% tween-20 to prevent non-specific interactions. Association and dissociation times were 900 s, respectively. To evaluate CD3 binding, anti-human IgG Fc (AHC) capture biosensors (Sartorius) were loaded with 3 ug/mL bispecific sample of interest in PBS. After loading sensor tips were washed in 1×DPBS+0.02% Tween 20+1 mg/mL Bovine Serum Albumin (BSA) for blocking. Recombinant CD3 antigen were loaded to antibody coated sensors at 7 concentrations, starting at 100 nM in 2-fold dilutions (100 nM˜1.5 nM), diluted in 1×DPBS with 0.02% tween 20 and 1 mg/mL BSA to prevent non-specific interactions. Association was monitored for 1800 s and dissociation for 900 s, respectively. All measurements were performed at 30° C. with agitation at 1,200 rpm. Sensorgrams were referenced for buffer effects and then analyzed using the FORTEBIO Data Analysis HT Software (V. 12.0.1.55). Kinetic responses were baseline subtracted, aligned and globally fit using a 1:1 fitting model or 2:1 heterogeneous ligand binding model to obtain values for association (Kon), dissociation (Koff) rate constants and the equilibrium dissociation constants (KD).

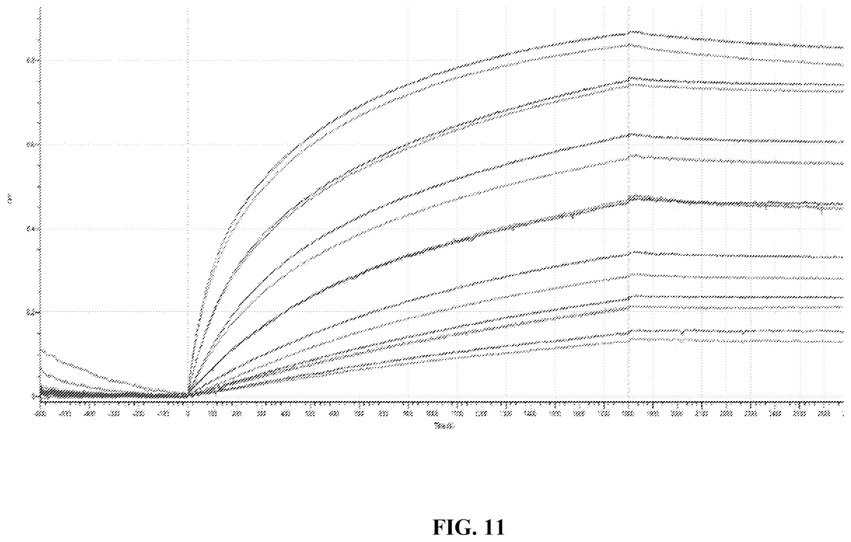

To determine whether the stapling impacts binding to target antigens of interest on either the scFv or Fab containing arms, binding was performed using bio-layer interferometry (BLI) and ELISA with highly purified bispecific samples (TD01B46, B48 and B49). Recombinant CD3E/D heterodimer protein was used to assess binding of the scFv/spFv anti-CD3 arms. Binding measurements showed that the scFv and spFv bi-specifics bind CD3 similarly, indicating incorporation of ‘stapling’ to the scFv did not alter CD3 binding (FIG. 11, BLI sensorgrams). The sensograms from BLI were well fit with a heterogeneous binding mode, but poorly with a simple 1:1 binding mode. The kinetic values (KD1/KD2, ka1/ka2, kd1/kd2) showed the stapled anti-CD3 molecule had comparable if not slightly enhanced affinity to either scFv molecule (Table 11). These data indicate that the spFv retains binding affinity of the corresponding scFv proteins.

TABLE 11
CD3 binding of scFv and spFv bispecific molecules by
BLI, using 2:1 heterogenous ligand fitting model.

| Sample Name | KD1 (M) | KD2 (M) | ka (1/Ms) | ka2 (1/Ms) | kdis (1/s) | kdis2 (1/s) |
|---|---|---|---|---|---|---|
| Cris7 scFv Bird | 1.27E−10 | 3.42E−09 | 1.60E+05 | 1.64E+04 | 2.04E−05 | 5.61E−05 |
| (B49) | | | | | | |
| Cris7 spFv (B46) | 2.22E−11 | 2.99E−09 | 1.58E+05 | 1.56E+04 | 3.49E−06 | 4.65E−05 |
| Cris7 scFv B48 | 2.38E−10 | 4.62E−09 | 1.47E+05 | 1.61E+04 | 3.49E−05 | 7.45E−05 |
| G4S | | | | | | |

### 6.2.7 Cytotoxicity Assay

To determine the effect of BCMA targeting test molecules upon T cell activation and killing potential of tumor cells, the Intellicyt iQue3 and ForeCyt software (Sartorius) was utilized. H929-Fluc-GFP cells served as target cells for two human donor Pan T cells (Hemacare). The assay was set up in a 96-well plate at a T cell to target ratio of 3:1. Test molecules were added at a starting concentration of 10 nM and serially diluted at 1:4 in complete media. All molecules were tested in duplicate at minimum. Detection of killing and T cell activation status was assessed 72 hrs later by flow cytometry. Endogenous GFP expressed in H929 was used to separate T cells from target cells. Cytotoxicity was measured using Near-IR Live/Dead stain (ThermoFisher), while activation in CD4 and CD8 T cells was assessed with anti-human CD25-BV650 (BD Biosciences), anti-human CD4-BV510 (Biolegend) and anti-human CD8-PE/Cy7 (Biolegend). Using Prism software (Graphpad), cytotoxicity and CD25 MFI data was exported, log-transformed, and 4 parameter logistically fit to generate regression curves for reporting of EC50.

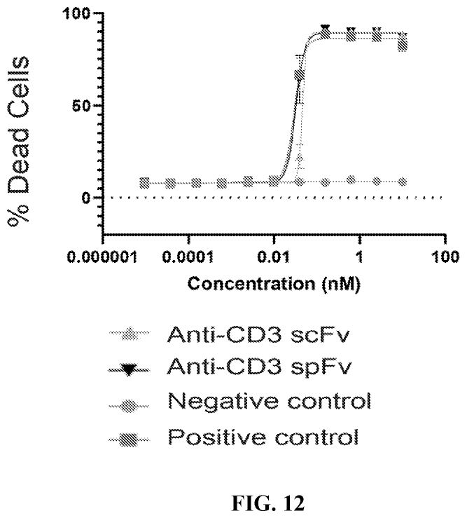

To determine the effect of BCMA targeting bispecific molecules upon T cell activation and killing potential of tumor cells, H929-Fluc-GFP cells served as target cells for two human donor pan-T cells. Detection of killing and T cell activation status was assessed 72 h later by flow cytometry. All bispecific proteins potently killed BCMA+H929−GFP+ cells in a cytotoxicity assay with very similar EC50 (data not shown), whereas a negative control bispecific with a Cris7b scFv/non-targeting Fab (CD8B24) showed no killing activity. All bispecific constructs containing either an scFv or spFv domain also activated CD4⁺ and CD8⁺ T cells with similar EC50s whereas the negative control molecule did not activate T cells (data now shown). This supports that the H929 cell killing was the result of T cell activation by CD3 redirecting. Taken together, spFv fully retained the scFv function in therapeutic constructs. As shown in FIG. 12, scFv and spFv bispecifics had similar CD3-mediated killing properties.

### 6.3 Example 3: “Stapling” scFv for Biotherapeutics Having Superior Properties

In this example, a simple and widely applicable scFv “stapling” strategy was provided that significantly improves stability compared with its unstapled version. The “stapled” scFv molecules (spFv) generally have an increase of about 10° C. in Tm for scFv molecules with both kappa and lambda light chains. In a set of anti-CD3 scFv/spFv and anti-BCMA Fab bispecific molecules, the results showed that stapling the anti-CD3 scFv significantly improved the yields and quality of the bispecific monomer, whereas some scFv containing bispecifics were a mixture of monomer and oligomers. The spFv retained binding affinity to CD3 compared with the corresponding scFv-containing bispecifics. The scFv and spFv bispecific proteins activated CD4+ and CD8+ equally with similar killing of BCMA+ tumor cells. The spFv containing bispecifics also displayed minimal aggregation upon heat stress at high concentrations, whereas the corresponding scFv molecules displayed significant aggregation. This was also true for a number of other spFv containing bi- and tri-specifics proteins. Thus, spFv can lead to equally potent biotherapeutics such as bi-, multispecifics with significantly improved developability. Further, stapling can also increase the success of scFv conversion, thus allowing more scFv molecules to be available as molecular building blocks for therapeutic constructs.

### 6.3.1 Materials & Methods

6.3.1.1 Expression and Purification of CAT2200 scFv/spFv

All scFv and spFv molecules except CAT2200a scFv LH were cloned into a CMV promoter driven mammalian expression vector. These constructs were transfection into Expi293 cells using manufacturer protocols and cells were cultured for 5 days. Each protein was purified from the clarified supernatant on 1 ml His-TRAP HP columns (GE Healthcare) via an AKTAXPRESS system (GE Healthcare). The column was prepared with a gradient of 0-100% Elution Buffer (Wash Buffer: 50 mM Tris, pH 7.5, 500 mM NaCl, 20 mM Imidazole; Elution Buffer: 50 mM Tris, pH 7.5, 500 mM NaCl, 500 mM Imidazole) to remove loosely bound nickel and then re-equilibration in DPBS. The cleared supernatant was first adjusted to 50 mM Tris, pH 7.5 and 20 mM imidazole and then loaded over 1 mL HisTrap HP column at 4° C. 0.8 mL/min. The column was then washed with PBS until stable baseline was obtained. Then the column was further washed with 20 CV of Wash Buffer, eluted with Elution buffer into a single injection loop and desalted in 1×DPBS over 26/10 HIPREP Desalting Column and fractions collected. Fractions containing the purified protein were then pooled and concentered. The B18 scFv and spFv proteins were dialyzed into DPBS for thermal stability measurements (DSC and NanoDSF) and 25 mM Tris, pH 7.5 and 100 mM NaCl for other studies. The other scFv and spFv proteins were dialyzed in 25 mM MES, pH 6.0 and 100 mM NaCl.

CAT2200a scFv LH was purchased from Sino Biological, which was produced in HEK293. Concentration was 0.77 mg/mL in DPBS, pH 7.2. A mutant of IL-17 (12-132 with K70Q A132Q C106S mutations, IL-17 hereafter for simplicity) was purchased from Accelagen (CA). The protein was refolded from E. coli inclusion body following their proprietary refolding protocol and provided at 1.50 mg/mL in 20 mM NaCl, 20 mM MES, pH 6.0.

6.3.1.2 Expression and Purification of Anti-CD3 Cris7a and Cris7b scFv/spFv

All scFv and spFv molecules were cloned into a CMV promoter driven mammalian expression vector. To ‘humanize’ the BCMB749 Fab arm sequence, CDRs, using AbM definition, were grafted onto human VL and VH with incorporated back mutations to mouse parental sequence. These constructs were transfected into Expi293 cells using manufacturer protocols and cells were cultured for 5 days. Each protein was purified from the clarified supernatant on 1 ml His-TRAP HP columns (GE Healthcare) via an AKTAXPRESS system (GE Healthcare). The column was prepared with a gradient of 0-100% Elution Buffer (Wash Buffer: 50 mM Tris, pH 7.5, 500 mM NaCl, 20 mM Imidazole; Elution Buffer: 50 mM Tris, pH 7.5, 500 mM NaCl, 500 mM Imidazole) to remove loosely bound nickel and then re-equilibration in DPBS. The cleared supernatant was first adjusted to 50 mM Tris, pH 7.5 and 20 mM imidazole and then loaded over 1 mL HisTrap HP column at 4° C. 0.8 mL/min. The column was then washed with PBS until stable baseline was obtained. Then the column was further washed with 20 CV of Wash Buffer, eluted with Elution buffer into a single injection loop and desalted in 1×DPBS over 26/10 HiPrep Desalting Column and fractions collected. Fractions containing the purified protein were then pooled and concentrated. The scFv and spFv proteins were dialyzed into DPBS for thermal stability measurements.

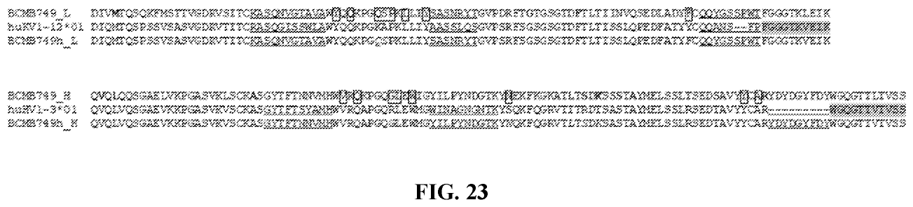

FIG. 23 shows humanization and sequence alignment of BCMB749. Each sequence alignment contained the parental (top), selected human acceptor germline sequence (middle) and the CDR-grafted with back mutations italicized (bottom). CDRs are underlined. Bold: CDR support positions in the framework regions. Boxed: VL/VH interface residues. For the humanization methods, the mouse parental sequences of the VH and VL domains were annotated as shown in the sequence alignment in FIG. 23. CDRs were defined according to the AbM convention and were underlined. Framework positions were classified into CDR support (in bold font) and VL/VH interface (boxed). The parental sequence was aligned against human germline sequences and one VH and one VL were selected according to sequence identity. For VL, human IGKV1-12*01 was chosen and IGHV1-3*01 for the VH. The selected J segment was highlighted in gray based on sequence identity. In the sequence alignment (VL upper panel and VH lower panel), the first sequence was mouse parental, middle one was the chosen human germline acceptor and the bottom one was the humanized variant. To generate the humanized variant, CDRs as defined above were grafted into the corresponding regions in the acceptor human GLs. Positions that were classified as CDR support and/or VL/VH interfaces were then back mutated to their parental amino acids if they were different between the parental and human acceptors. There were three and five back mutations in the humanized VL and VH domains, respectively.

### 6.3.1.3 SDS-PAGE Analysis of Protein Samples

Cris7a and Cris7b scFv and spFv domain proteins were prepared at 0.3 mg/mL in 1×dPBS with the addition of 1×NuPAGE LDS Sample Buffer (Invitrogen). Samples were split in half and to one set 1 mM DTT was added to the samples to allow for reduction. All samples were heated at 90° C., 5 m. prior to loading. Twenty microliters of all samples were loaded into 4-12% Bis-Tris NuPAGE Gel (Invitrogen), along with SEEBLUE PLUS2 Pre-stained Ladder (Invitrogen) and run at 180V for 45 min. The final gel was stained with SIMPLYBLUE SafeStain (Invitrogen) for 1 h, RT and then destained overnight in ddH20.

### 6.3.1.4 Small Scale Expression and Purification of CD3/BCMA Bispecifics.

All bispecific scFv and spFv molecules, along with corresponding Fab HC and LC arms, were cloned into a CMV promoter driven mammalian expression vector. These constructs were transfected into ExpiCHO cells using manufacturer protocols and cells were cultured for 7 days. All samples were harvested and resulting supernatant was filtered through a 0.2 mm filter for clarification. Samples were purified through a two-step process. Clarified supernatants were first passed over a HITRAP MABSELECT SURE (Protein A) 1 mL column (GE Healthcare). The column was equilibrated and washed in 1×DPBS; final samples were eluted in 0.1 M sodium acetate pH 3.5. Once the protein was eluted, samples were immediately neutralized with 2.5M Tris-HCl pH 7.5, and then loaded over 0.5 mL CAPTURESELECT CH1-XL Affinity Matrix (ThermoFisher). CH1 column was equilibrated and washed in 1×DPBS, and final samples were eluted in 0.1 M sodium acetate pH 3.5. Samples were dialyzed into 1×DPBS for storage, and 30 mL of sample was assessed for sample quality and purity on Agilent AdvanceBio SEC 300A (Column) (Agilent) using 1×DPBS as running buffer. Final samples were assessed to determine concentration by A280 and stored at 4° C.

### 6.3.1.5 Large Scale Protein Expression and Purification

Three of the bispecific proteins (TD01B46, TD01B48 and TD01B49) were further expressed at a larger scale expression at (500 ml or 1 L) to generate enough proteins for concentration and aggregation studies. Proteins were expressed as described above at larger volumes. These proteins were purified to high homogeneity using a slightly different process as follows. Half a liter of clarified cell culture supernatants of select samples were loaded onto pre-equilibrated 5 mL prepacked HITRAP MABSELECT PRISMA columns (Cytiva) with 1×dPBS (pH 7.2) on Akta Pure System (GE Healthcare). Columns were washed with 5 column volumes (CVs) of 1×dPBS (pH 7.2). Following the wash, 3-5 CVs of 100 mM sodium acetate (pH 3.5) were used to elute the bound antibodies. In line with collection of the enriched fractions post elution, proteins were neutralized with 15% (v/v) 2.5 M Tris-HCl (pH 7.5) using a mixing valve on the AKTA PURE system. The collected material was pooled together of each protein separately, and syringe-filtered with 0.2-μm filter. Samples were loaded onto pre-equilibrated 5 mL CaptureSelect CH1-XL prepacked affinity columns (Thermo Scientific) on AKTA XPRESS systems (Amersham Biosciences Corp.) and washed with 4 CVs of 1×dPBS (pH 7.2). The bound antibodies were then eluted off of the columns using 5 CVs of 100 mM sodium acetate (pH 3.5) and the enriched fractions were collected. The collected material was neutralized to final volume of 15% (v/v) 2.5 M Tris-HCl (pH 7.5) and syringe-filtered with 0.2-μm filters. Samples were diluted 8-10-fold with 20 mM MES (pH 5.5) and loaded onto 180 mL CAPTO S IMPACT column (Cytiva) using AKTA PURE System (GE Healthcare) which was equilibrated with 20 mM MES (pH 5.5). The column was washed with 1 CV of 20 mM MES (pH 5.5) and 3 CVs of 20 mM MES (pH 6.5) to remove loosely bound impurities. The proteins were eluted with 15 CVs of buffer B (20 mM MES, 1M sodium chloride, pH 6.5) over a linear gradient (0-30% buffer B). Fractions were pooled and passed through 0.2-μm filters. All samples were loaded onto a pre-equilibrated with 1×dPBS (pH 7.2) 120 mL SUPERDEX 200 pg SEC column (Cytiva) using AKTA AVANT System (GE Healthcare). Proteins were eluted off the column with 1.5 CVs 1×dPBS (pH 7.2). The concentration of purified protein was determined by absorbance at 280 nm with 0.1 and 0.7 mm pathlengths on a DROPSENSE spectrophotometer. The quality of the purified protein was assessed by SDS-PAGE (3.5 ug of sample, Invitrogen, NuPage 4-12% Bis-Tris) and analytical size exclusion HPLC (20 ug sample; column: TSKgel BIOASSIST G3SW×1, 7.8 mm ID×30 cm H, 5 μm, TOSOH; guard column: TSKgel BIOASSIST SW×1 guard column, 6 mm ID×4 cm H, 7 um, TOSOH; Agilent HPLC system) at 1 mL/min for 20 min using 200 mM sodium phosphate (pH 6.8) as the running buffer. The endotoxin level was measured using a turbidometric LAL assay (PYROTELL-T, Associates of Cape Cod; Falmouth, MA).

### 6.3.1.6 Differential Scanning Calorimetry (DSC)

The scFv and spFv proteins were dialyzed overnight against 1×DPBS (Gibco) for GLk1 and CAT2200a/CAT2200b or MES (25 mM MES, pH 6.0, 100 mM NaCl) for GLk2. Dialysis buffer was then 0.22 micron filtered and used as the reference solution and for buffer-buffer blanks in the DSC experiment. Proteins were diluted to ˜0.5 mg/mL in the filtered buffer and 400 μL of each protein or buffer sample was loaded into a 96-well plate (MicroLiter Analytical Supplies, 07-2100) and kept at 4° C. in the autosampler drawer over the course of the experiment. A MICROCAL Capillary DSC with Autosampler (Malvern) was used to perform the DSC experiments. DSC scans were performed from 25-95° C. at a 60° C./h scan rate with no sample rescans. No feedback was selected and the filtering period was set at 15 s. After each sample, cells were cleaned with a 10% Contrad-70 solution and a buffer-buffer blank was run. Data analysis was performed using Origin 7.0 with the MICROCAL VP-Capillary DSC Automated Analysis add-on (Malvern). The baseline range and type were manually chosen and then subtracted. The previous buffer blank was subtracted from the sample curve followed by concentration-dependent normalization. The thermal melting profiles were analyzed using the non-two-state transition model. Iterative curve fitting was performed to derive thermodynamic parameters associated with the melting, e.g., thermal stability, enthalpy, which are reported in Table 12.

TABLE 12
Thermal stability (DSC) of scFv and spFv of test molecules Glk1, Glk2, CAT2200
and Cris7 variants. ΔTm = Tm(spFv) − Tm(scFv).

| | | Linker | Tm | Tm Error | | ΔH | ΔH Error | ΔH |
|---|---|---|---|---|---|---|---|---|
| Molecule name | Orientation | (n + 4 + m) | (° C.) | (° C.) | ΔTm | (kcal/mol) | (kcal/mol) | ratio |
| GLk1 scFv | LH | 4 × G4S | 71.2 | 0.07 | | 124.5 | 2.7 | |
| GLk1 spFv | LH | 9 + 4 + 5 | 80.2 | 0.05 | 9.0 | 160.5 | 3.2 | 1.29 |
| GLk1 scFv | HL | 4 × G4S | 70.1 | 0.05 | | 134.5 | 2.0 | |
| GLk1 spFv | HL | 9 + 4 + 5 | 79.1 | 0.04 | 9.1 | 160.5 | 2.6 | 1.19 |
| Glk2 scFv | LH | 4 × G4S | 57.9 | 0.03 | | 129.5 | 1.6 | |
| Glk2 spFv | LH | 9 + 4 + 5 | 68.6 | 0.02 | 10.7 | 148.0 | 1.7 | 1.14 |
| Glk2 scFv | HL | 4 × G4S | 57.3 | 0.03 | | 130.0 | 1.5 | |
| Glk2 spFv | HL | 6 + 4 + 6 | 64.7 | 0.02 | 7.5 | 140.5 | 1.7 | 1.08 |
| CAT2200b scFv | LH | 4 × G4S | 57.2 | 0.04 | | 91.2 | 1.6 | |
| CAT2200a spFv | LH | 9 + 4 + 4 | 68.8 | 0.04 | 11.6 | 143.5 | 2.5 | 1.57 |
| CAT2200a scFv | HL | 4 × G4S | 55.9 | 0.08 | | 97.4 | 2.7 | |
| CAT2200b spFv | HL | 9 + 4 + 4 | 67.4 | 0.04 | 11.5 | 151.5 | 2.7 | 1.56 |
| Cris7a scFv | LH | 4 × G4S | 59.7 | 0.04 | | 124.0 | 2.4 | |
| Cris7a spFv | LH | 8 + 4 + 4 | 71.6 | 0.03 | 11.9 | 177.0 | 2.4 | 1.43 |
| Cris7b scFv | LH | 4 × G4S | 57.0 | 0.05 | | 112.0 | 2.3 | |
| Cris7b spFv | LH | 8 + 4 + 4 | 68.6 | 0.03 | 11.5 | 176.0 | 2.5 | 1.57 |

### 6.3.1.7 Thermal Stability by UNCLE

Protein stability of several scFv/spFv paired samples were evaluated incorporating intrinsic fluorescence and static light scattering (SLS) analyses on the UNCLE instrument (Unchained Labs, Pleasanton, CA, USA). Thermal melting mid-point (Tm) was determined by intrinsic fluorescence measured through with blue wavelength acquisition (Blue—473 nm, filter 4) and thermal aggregation (Tagg) by SLS through UV acquisition (UV—226 nm, filter 3) settings. Samples concentrations ranged from 0.1-0.5 mg/mL in DPBS, and analysis was performed in duplicate using 8.8 μL sample volume in the UNCLE Uni cuvettes. Thermal melt profiles were collected using a linear heating ramp of 0.3° C./minute between 20˜85° C., with a 60 s incubation period and 30 s plate hold period. Data was collected and analyzed concurrently with Uncle software and Tm and Tagg (Tonset) values were directly exported.

6.3.1.8 Crystallization of Unbound scFv and spFv

Proteins were concentrated in their respective buffers: GLk1 spFv VL-VH to 8.67 mg/ml in 25 mM MES, pH 6.0, 100 mM NaCl; GLk1 spFv VH-VL to 5 mg/ml in 25 mM MES, pH 6.0, 100 mM NaCl; GLk2 spFv VH-VL to 8.66 mg/ml in 25 mM Tris, pH7.5, 100 mM NaCl; cat 2200b spFv VH-VL to 25 mM MES, pH 6.0, 100 mM NaCl. Crystallization was set up for each protein in sitting drop format in Corning 3550 crystal trays using a MOSQUITO robot. Each well contained 100 nl of protein and 100 nl of reservoir solution and incubated against 70 ul of reservoir at 20° C. The reservoir solutions were IH1 and IH2 custom conditions as well as PEG Ion Screen HT (Hampton Research). Some initial conditions were refined by varying reservoir components in optimization attempts. Diffraction quality crystals were obtained for some of scFv and spFv proteins. Crystals were soaked for a few seconds in the mother liquor supplemented with 20% glycerol and flash frozen in liquid N2. X-ray data were collected at IMCA-CAT Beamline 171D at Argonne National Lab.

6.3.1.9 Crystallization of CAT2200a scFv VL-VH and CAT2200a spFv VL-VH in Complex with IL-17

The IL-17/CAT2200a scFv VL-VH complex was generated by mixing 333 μL of IL17 (1.5 mg/ml) with 1.74 ml of Cat 2200a scFv (0.69 mg/mL) and incubating for 3 hours at 4° C. The mixture was concentrated with 10 kDa cutoff AMICON Ultra concentrator to about 400 μL and loaded onto a SUPERDEX75 column equilibrated in 250 mM NaCl, 20 mM HEPES, pH 7.5. The fractions corresponding to the complex were pooled and concentrated to a volume of 150 μL. The sample was diluted and concentrated 4 times: addition of 350 μL 50 mM NaCl, 20 mM HEPES, pH 7.5 and concentration to just under 150 μL. The volume was brought to ˜105 μL and concentration determined to be 2.69 mg/mL. Crystallization was set up in a sitting drop format using a MOSQUITO crystallization robot with 150 nL protein+150 nL reservoir in Corning3550 plates against 80 μL reservoir, which was a set of buffer and precipitant conditions pre-formulated in-house. The plates were incubated at 20° C. One of conditions (Na Acetate, pH 4.5, 25% PEG 3K, 0.2 M Am Acetate) produced very small crystals. These were harvested and turned into crystallization seeds using Hampton Seed Bead in 100 μL 27% PEG 3350, 200 mM ammonium acetate, 100 mM sodium acetate, pH 4.5 in a Hampton Seed Bead tube.

Diffraction quality crystals were obtained by the same procedure except with the addition of the seeds above: 150 nL protein+100 nL reservoir+50 μL seeds. Crystals grew from 0.1 M Tris 8.5, 18% PEG3K, 0.2M LiSO4 and were transferred to a synthetic mother liquor (0.1 M Tris, pH 8.5, 10% PEG 3350, 0.2 M LiSO4 and 20% glycerol) and flash frozen in liquid nitrogen. X-ray diffraction data were collected at IMCA-CAT ID17 at Argonne National Laboratory.

The IL-17/CAT2200a spFv VL-VH complex were generated by mixing 167 μl of IL-17 (250 μg) with 154 μl MSCW274 (467 μg in 250 mM NaCl, 20 mM MES, pH 6.5) and incubating at 4° C. overnight. The mixture was concentrated in a 10 kDa MWCO Amicon Ultra 0.5 mL concentrator to ˜100 uL, then repeatedly diluted and concentrated 5 times: concentrate to ˜150 μL and added 350 μL 50 mM NaCl, 20 mM HEPES, pH 7.5. The final volume was 100 μL and the concentration of the complex was determined to be 6.0 mg/ml. Crystallization was set up similarly as for scFv/IL-17 complex in sitting drops using the MOSQUITO robot. The sitting drop were composed of 150 nL protein+120 nL reservoir+30 nL seeds (scFv/IL-17 above). The reservoir solution were a set of conditions varying PEG 3350 concentration and salts. The crystallization plates were incubated at 20° C. Small crystals were obtained from 15.5% PEG 3350, 0.4 M NaH2PO4. Crystals were transferred into 16% PEG 3350, 0.2 M NaH2PO4, 20% Glycerol, and flash frozen LN2. X-ray diffraction data were collected at IMCA-CAT ID17 at Argonne National Laboratory.

All X-ray diffraction data were processed with XDS (Kabsch et al. Acta crystallographica. Section D, Biological crystallography 66, 125-132 (2010)) and CCP4 (Collaborative Computational Project, N. The CCP4 suite: programs for protein crystallography. Acta crystallographica. Section D, Biological crystallography 53, 240-255 (1994)). All crystal structures were solved by molecular replacement (MR) using Phaser (Read et al. Acta crystallographica. Section D, Biological crystallography 57, 1373-1382 (2001).) with homology models generated in MOE (Montreal, Canada) except for scFv CAT2200a scFv VL-VH/IL-17 complex, for which the structure of pdb id 2vxs (Gerhardt et al. Journal of molecular biology 394, 905-921 (2009)) was used as search models. The structural models were refined in PHENIX (Adams et al. J Synchrotron Radiat 11, 53-55 (2004)) and manually adjusted in COOT (Emsley et al. Acta crystallographica. Section D, Biological crystallography 66, 486-501 (2010)). Molecular graphics figures were generated in PyMol (schrodinger website). The X-ray diffraction data and refinement statistics are given in Table 13. Atomic coordinates for Glk1spFv_LH(mscg380) and Glk1spFv_HL(mscg385) are shown in Table 18 and Table 19 respectively. Atomic coordinates for Glk2spFv_HL(mscg274) are shown in Table 20. Atomic coordinates for CATspFv_HL (mscg379) are shown in Table 21. Atomic coordinates for CATscFv_LH/IL-17 (scFv-Cat2200a) are shown in Table 22. Atomic coordinates for CATspFv_LH/IL-17 (mscw374) are shown in Table 23.

TABLE 13
X-ray data collection and refinement statistics

| | Glk1spFv_{—} | | Glk1spFv_{—} | |
|---|---|---|---|---|
| Data Collection | LH(mscg380) | | HL(mscg385) | |
| X-ray source | APS 17-ID | | APS 17 ID | |
| Wavelength (Å) | 1.000 | | 1.000 | |
| Space group | P2₁2₁2₁ | | P1 | |
| Unit cell axes (Å), | 45.63, 78.94, | | 37.94, 50.74, | |
| | 257.30 | | 63.20 | |
| angles (°) | 90, 90, 90 | | 76.35, 89.20, | |
| | | | 69.16 | |
| Resolution (Å) ^{a} | 128.65-1.65 | | 61.23-2.10 | |
| | (1.84-1.65)* | | (2.15-2.10) | |
| No. measured reflections | 338,834 | (14,700) | 123,954 | (6,230) |
| No. unique reflections^{a} | 54,237 | (2,712) | 24,805 | (1,840) |
| <I/σ> | 13.9 | (2.0) | 7.4 | (2.3) |
| Completeness (%)^{a} | 47.8 | (8.8)* | 99.5 | (99.2) |
| Redundancy^{a} | 9.5 | (9.9) | 5.0 | (3.5) |
| R_{sym}^{a, c} | 0.081 | (0.955) | 0.167 | (0.721) |
| Rₚᵢₘ^{a, d} | 0.035 | (0.431) | 0.069 | (0.390) |
| CC_{1/2}^{a, e} | 1.00 | (0.67) | 0.99 | (0.83) |
| No. molecules per ASU ^{f} | 4 | | 2 | |
| Structure Refinement | | | | |
| Resolution (Å) | 48.80-1.65 | | 35.35-2.10 | |
| | (1.68-1.65)* | | (2.80-2.10) | |
| No. reflections in | 54,216 | (139) | 24,789 | (2,592) |
| refinement | | | | |
| Number of atoms | 7,544 | | 3,744 | |
| Number of solvent atoms | 425 | | 180 | |
| R_{cryst}^{a, g} | 0.211 | (0.441) | 0.182 | (0.257) |
| R_{free}^{a, h} | 0.248 | (0.666) | 0.224 | (0.328) |

| RMSD bond lengths (Å) | 0.011 | 0.007 |
|---|---|---|
| RMSD bond angles (°) | 1.37 | 0.93 |
| Mean B factors (Å²) | | |
| Proteins | 28.9 | 34.6 |
| Solvent | 32.8 | 37.1 |
| Ramachadran plot ⁱ | | |
| Favored (%) | 96.7 | 97.5 |
| Outliers (%) | 0.5 | 0.0 |
| All atom clash score | 6.9 | 5.2 |
| PDB ID | 8DY2 | 8DY0 |

| | Glk2spFv_{—} | | CATspFv_{—} | |
|---|---|---|---|---|
| Data Collection | HL(mscg274) | | HL(mscg379) | |
| X-ray source | APS 17-ID | | APS 17 ID | |
| Wavelength (Å) | 1.000 | | 1.000 | |
| Space group | P4₁2₁2 | | P3₁21 | |
| Unit cell axes (Å), | 63.03, 63.03, | | 63.34, 63.34, | |
| | 124.96 | | 104.58 | |
| angles (°) | 90, 90, 90 | | 90, 90, 120 | |
| Resolution (Å)^{a} | 56.28-1.51 | | 48.59-2.40 | |
| | (1.65-1.51)* | | (2.49-2.40) | |
| No. measured reflections | 381,408 | (13,980) | 95,063 | (9,273) |
| No. unique reflections^{a} | 30,532 | (1,527) | 9,925 | (980) |
| <I/σ> | 17.3 | (1.5) | 13.2 | (2.0) |
| Completeness (%)^{a} | 75.5 | (16.0)** | 99.8 | (99.6) |
| Redundancy^{a} | 12.5 | (9.2) | 9.6 | (9.5) |
| R_{sym}^{a, c} | 0.097 | (1.777) | 0.081 | (1.294) |
| Rₚᵢₘ^{a, d} | 0.039 | (0.776) | 0.026 | (0.417) |
| CC_{1/2}^{a, e} | 1.00 | (0.51) | 1.00 | (0.99) |
| No. molecules per ASU ^{f} | 1 | | 1 | |
| Structure Refinement | | | | |
| Resolution (Å) | 56.28-1.51 | | 34.86-2.40 | |
| | (1.56-1.51) | | (2.75-2.40) | |
| No. reflections in refinement | 30,529 | (166) | 9,683 | (2,967) |
| Number of atoms | 2,096 | | 1,743 | |
| Number of solvent atoms | 289 | | 0 | |
| R_{cryst}^{a, g} | 0.183 | (0.209) | 0.262 | (0.507) |
| R_{free}^{a, h} | 0.274 | (0.431) | 0.328 | (0.623) |

| RMSD bond lengths (Å) | 0.007 | 0.002 |
|---|---|---|
| RMSD bond angles (°) | 0.92 | 0.49 |
| Mean B factors (Å²) | | |
| Proteins | 22.7 | 123.3 |
| Solvent | 34.4 | |
| Ramachadran plot ⁱ | | |
| Favored (%) | 97.5 | 93.4 |
| Outliers (%) | 0.0 | 0.0 |
| All atom clash score | 2.3 | 7.1 |
| PDB ID | 8DY3 | 8DY4 |

| | CATscFv_{—} | | CATspFv_{—} | |
|---|---|---|---|---|
| | LH/IL-17 | | LH/IL-17 | |
| Data Collection | (scFv-Cat2200a) | | (mscw374) | |
| X-ray source | APS 17-ID | | APS 17 ID | |
| Wavelength (Å) | 1.000 | | 1.000 | |
| Space group | P2₁ | | C222₁ | |
| Unit cell axes (Å), | 51.93, 62.05, | | 82.43, 226.38, | |
| | 111.71 | | 75.35 | |
| angles (°) | 90, 99.69, 90 | | 90, 90, 90 | |
| Resolution (Å)^{a} | 43.70-2.68 | | 45.26-2.20 | |
| | (2.74-2.68) | | (2.28-2.20) | |
| No. measured reflections | 59,277 | (4,148) | 179,538 | (18,221) |
| No. unique reflections^{a} | 18,382 | (1,214) | 36,090 | (3,572) |
| <I/σ> | 11.7 | (2.2) | 7.2 | (0.9) |
| Completeness (%)^{a} | 92.3 | (91.6) | 99.5 | (99.7) |
| Redundancy^{a} | 3.2 | (3.4) | 5.0 | (5.1) |
| R_{sym}^{a, c} | 0.080 | (0.669) | 0.134 | (1.148) |
| Rₚᵢₘ^{a, d} | 0.044 | (0.358) | 0.058 | (0.494) |
| CC_{1/2}^{a, e} | 1.00 | (0.91) | 1.00 | (0.72) |
| No. molecules per ASU ^{f} | 1 | | 1 | |
| Structure Refinement | | | | |
| Resolution (Å) | 43.69-2.68 | | 45.25-2.20 | |
| | (2.85-2.68) | | (2.26-2.20) | |
| No. reflections in refinement | 18,344 | (3,002) | 36,069 | (2,599) |
| Number of atoms | 4,933 | | 5,326 | |
| Number of solvent atoms | 61 | | 295 | |
| R_{cryst}^{a, g} | 0.222 | (0.311) | 0.188 | (0.260) |
| R_{free}^{a, h} | 0.266 | (0.395) | 0.234 | (0.301) |

| RMSD bond lengths (Å) | 0.002 | 0.007 |
|---|---|---|
| RMSD bond angles (°) | 0.51 | 0.90 |
| Mean B factors (Å²) | | |
| Proteins | 48.8 | 43.5 |
| Solvent | 45.3 | 43.6 |
| Ramachadran plot ⁱ | | |
| Favored (%) | 96.6 | 94.7 |
| Outliers (%) | 0.0 | 0.2 |
| All atom clash score | 6.1 | 3.6 |
| PDB ID | 8DY1 | 8DY5 |

*The resolution limits reported by based on Staraniso (see, e.g., staraniso.globalphasing.org) are 2.82, 1.84, 1.65 Å in ^{a}*, ^{b}* & ^{c}*, respectively. The ellipsoidal completeness is 88.6% (75.8%), respectively.
*The resolution limits reported by Staraniso (see, e.g., staraniso.globalphasing.org) are 1.70, 1.70, 1.51 Å in ^{a}*, ^{b}* & ^{c}*. The ellipsoidal completeness is 94.2% (68.8%), respectively.
^{a}Parentheses denote outer-shell statistics.
^{b} The resolutions based upon the anisotropic server (services.mbi.ucla.edu/anisoscale) are 3.05, 3.1 and 4.0 Å for the ^{a}*, ^{b}* and ^{c}* directions respectively. The diffraction data statistics here referred to the values after anisotropic data treatment at this web server.
^{c}R_{sym} = ΣₕₖₗΣᵢ |I_{hkl, ith} − <Iₕₖₗ>|/ΣₕₖₗΣ_{ihkl, i} and ^{d}Rₚᵢₘ = Σₕₖₗ[1/(N − 1)]^{1/2} Σᵢ|I_{hkl, i} − <Iₕₖₗ>|/ΣₕₖₗΣ_{ihkl, i} where I_{hkl, i} is the scaled intensity of the i measurement of reflection h, k, l, <Iₕₖₗ> is the average intensity for that reflection, and N is the redundancy.
^{e}CC_{1/2} = Pearson Correlation Coefficient between two random half datasets.
^{f} No. molecules refers to the number of scFv/spFv molecules or scFv/spFv/antigen complexes per asymmetric unit (ASU)
^{g}R_{cryst} = Σₕₖₗ|Fₒ − F_{c}|/Σₕₖₗ|Fₒ|, where Fₒ and F_{c} are the observed and calculated structure factors, respectively.
^{h}R_{free} was calculated as for R_{cryst}, but with 5% of data excluded before refinement or a maximum of 2000 reflections.
ⁱ Ramachadran plot and all atom clash scores were calculated with MolProbity (Zhang and Snyder J Biol Chem 264, 18472-18479 (1989)).

### 6.3.1.10 Disulfide Mapping Analysis: Sample Preparation, Instrument Parameters and Data Analysis

Non-reduced digestion of the bispecific molecule was performed using an enzyme combination of recombinant LysC (rLysC) and PROALANASE (Promega, Cat. #VA2161). The digestion protocol used was a modified version of the Promega ACCUMAP Low pH Protein Digestion protocol for nonreduced disulfide mapping protocol. The bispecific antibody (40 μg) was denatured using 8M Guanidine HCl and alkylated using 0.2 M N-ethylmaleimide (NEM, Promega, Cat #VB102A) at 37° C. for 30 min. 40 μg of denatured and alkylated protein was directly mixed with rLysC. The denatured and alkylated antibody (43 μl) was mixed with 25 μL of rLysC (Promega low pH resistant rLysC, Promega, Cat. #V167A) and incubated at 37° C. for 1 hr. The sample was desalted using Thermo Scientific Single-Use RED plate with inserts, in 0.1% TFA (aq) (pH 1.8). The sample was transferred to the sample holding well (˜67 μL) while in the dialysate well 325 μL of 0.1% TFA was added, and the dialysis plate was placed on a shaker at 37° C. for 40 min. This step was performed 3 times and the sample was recovered at the same concentration (˜40 μg). For ProAlanase digestion, the enzyme was added to an enzyme-to-protein ratio of 1:40 (w/w) and the digestion was incubated at 37° C. overnight. The pH of the samples was kept at ˜2 throughout the digestions to minimize potential disulfide scrambling induced by protein digestion at high pH^{44, 45}.

LC/MS peptide mapping data were acquired on a ThermoFisher Scientific (San Jose, CA) VANQUISH Dual UHPLC connected to an ORBITRAP Eclipse Mass Spectrometer. The HPLC column used was a Waters Acquity Premier CSH C18 2.1×150 mm heated at a temperature of 60° C. Peptides (˜15 μg of the sample) from the two-enzyme digestion were separated by a 120-minute gradient at a flow rate of 0.4 ml/min. The LC gradient consisted of an initial setting of 2% B (0.1% Formic acid/Acetonitrile) to 42% B in 114 minutes. The ORBITRAP Eclipse instrument was operated in ESI Positive mode using an Ion Max NG H-ESI source. Source settings were spray voltage of 3500 V, sheath gas (arb units) 35, aux gas (arb units) 7, sweep gas (arb units) 1. Ion transfer tube temperature and vaporizer temperature were 275° C. Instrument was operated in Data Dependent Acquisition (DDA) mode. MS1 parameters were orbitrap resolution of 120,000, scan range from 350-2000 m/z with RF lens of 30%. Dependent scan 1 was ddMS2 OT HCD (Higher-energy C-trap dissociation) with isolation window 1.6 m/z, collision energy fixed at 29%, orbitrap detection, resolution was 15,000 with a maximum injection time of 50 msec. Dependent scan 2 was ddMS2 OT ETD (Electron-transfer dissociation) with isolation window 1.6 m/z, ETD reaction time 110 ms, ETD reagent target 2.0e5, Max ETD reagent injection time was 200 ms. DDMS2 detection was set to orbitrap with resolution of 15,000 and a max injection time of 50 msec.

The LC-MS/MS files from the disulfide mapping experiments were analyzed using the disulfide workflow in Byonic (Protein Metrics, San Carlos, CA) software. The sequence of each chain and the expected disulfide links were manually entered. A semi-specific search with 3 miscleavages was used considering cleavage sites for both LysC and ProAlanase, including lysine, arginine, alanine, proline, serine, and glycine. For instrument parameters, 5 ppm was used as precursor mass tolerance and 20 ppm as the fragment mass tolerance, while Thermo scan headers was used for fragmentation type. Modifications such as oxidation on methionine and tryptophan, asparagine deamidation, free cysteine capping with NEM or cysteinylation were used as rare 1 fine controls. Glutamine to pyroglutamate on N-terminal glutamine was used as a common 1 fine control. Glycan modifications were not used, and the maximum precursor mass was set to 10 kDa. FDR (False discovery rate) of 1% was used and heavy multicore options were used for the data search. The search parameters were optimized to identify all expected disulfides. The properly cleaved disulfide connected peptides as well as semi-specific cleaved versions give rise to multiple peaks observed in the LC/MS Total Ion Chromatogram (TIC). Multiple criteria were used to verify true positives, such as high intensity peak in the extracted ion chromatogram (XIC), MS1 confirmation of the disulfide complex and MS2 sequence coverage.

### 6.3.1.11 Differential Scanning Fluorimetry (DSF) of Bispecific Variants

Conformational stability of bispecific proteins with Cris7b/CD3B219a99v scFv/spFv paired with two anti-BCMA Fab arms were measured using advanced differential scanning fluorimetry (nanoDSF) technology, by monitoring the intrinsic fluorescence of tryptophan upon thermal unfolding. The unfolding was measured by loading each sample into 24 well capillary (NanoTemper, Cat #PR-AC002) from a 384 well sample plate (ThermoNunc, Cat #264573), with a heating ramp of 1° C./minute between 20˜95° C. using the Prometheus NT.48 instrument (NanoTemper Technologies GmbH). Each sample was measured at 0.5 mg/ml in phosphate buffer saline (PBS) in duplicates. The intrinsic fluorescence of each sample at 330 and 350 nm was used to monitor unfolding during temperature ramp and recorded as changes in fluorescence intensity over time. Data were collected and saved as projects, processed using the PR.STABILITYANALYSIS v1.0.2 software. The processed data contained integrated thermal melting profiles, first derivatives for fluorescence at 330 nm, 350 nm, ratio 330/350, and light scattering data for all the samples. Thermal melting mid-point (Tm) values as well as onset of aggregation (Tagg) were identified and reported.

### 6.3.1.12 Bio-Layer Interferometry of Cris7 Bispecific Molecules

Binding of the Cris7b scFv and stapled spFv bispecific molecules to recombinant CD3 antigen (human CD3 epsilon and CD3 delta heterodimer protein, Acro Biosystems) and recombinant BCMA antigen were measured by biolayer interferometry (BLI) using an OCTET HTX instrument (Sartorius, formerly ForteBio). To evaluate BCMA binding, Streptavidin (SA) capture sensors (Sartorius) were loaded with biotinylated-BCMA protein to ˜1 nm signal in PBS. Bispecific samples were loaded to antigen coated SA sensors at 7 antibody concentrations starting at 100 nM in 2-fold dilution (100 nM˜1.5 nM), diluted in 1×DPBS with 0.05% tween-20 to prevent non-specific interactions. Association and dissociation times were 900 s, respectively. To evaluate CD3 binding, anti-human IgG Fc (AHC) capture biosensors (Sartorius) were loaded with 3 μg/mL bispecific sample of interest in PBS. After loading sensor tips were washed in 1×DPBS+0.02% Tween 20+1 mg/mL Bovine Serum Albumin (BSA) for blocking. Recombinant CD3 antigen were loaded to antibody coated sensors at 7 concentrations, starting at 100 nM in 2-fold dilutions (100 nM 1.5 nM), diluted in 1×DPBS with 0.02% tween 20 and 1 mg/mL BSA to prevent non-specific interactions. Association was monitored for 1800 s and dissociation for 900 s, respectively. All measurements were performed at 30° C. with agitation at 1,200 rpm. Sensorgrams were referenced for buffer effects and then analyzed using the ForteBio Data Analysis HT Software (V. 12.0.1.55). Kinetic responses were baseline subtracted, aligned and globally fit using a 1:1 fitting model or 2:1 heterogeneous ligand binding model to obtain values for association (Kon), dissociation (Koff) rate constants and the equilibrium dissociation constants (KD).

### 6.3.1.13 ELISA of Bispecific Molecules

Bispecific antibodies were analyzed for binding to either recombinant biotinylated BCMA or recombinant biotinylated CD3 antigen (human CD3 epsilon and CD3 delta heterodimer protein, Acro Biosystems). ELISAs were carried out according to standard protocols and plates were washed three time with TBS containing 0.05% Tween 20 (TBS-T) between each incubation step. Ninety-six-well MAXISORP plates (Nunc) were coated with 1 ug/mL of Streptavidin, diluted in 1×dPBS for 18 hours at 4° C. All plates were coated with 20 nM of antigen of interest for 1 hour at room temperature and then blocked with 3% BSA in PBS-T (1×dPBs with 0.05% tween 20) for 1 hour at room temperature. After blocking, plates were incubated with serial dilution of purified bispecific samples in PBS-T for 1 h at RT, and were then incubated with goat anti-human IgG F(ab′2)2 HRP (Jackson ImmunoResearch) for 1 hour at room temperature. After washing, BM Chemilluminesence ELISA Substrate (POD) was added (Millipore) and plates were immediately read on an ENVISION plate reader (Perkin Elmer) using ultrasensitive illuminesence. Raw data were exported to GRAPHPAD PRISM, where curves were generated and analyzed with a nonlinear regression curve fit.

### 6.3.1.14 High Concentration and Heat Stress Study

Concentratability studies were performed using Amicon Ultra 4 centrifugal filtration devices with 30 kDa MWCO membrane (Catalog #UFC803096. MilliporeSigma, Burlington MA). First, the spin columns were filled with water and spun at 4200 g for 6 min to equilibrate the membrane. TD01B46 (2 mg/ml) and TD01B49 proteins (0.5 mg/ml) were each loaded in pre-washed spin columns and centrifuged at 4200 g at 15-minute time intervals. At the end of each 15-minute centrifugation step, the concentrators were removed from the centrifuge and a visual estimate of the remaining sample volume was recorded. The concentration step was repeated until enough volume of the concentrated sample was available for profiling by SEC for purity analysis. At the end of the centrifugation process, the concentrated samples were recovered, and the protein concentration was determined using NANODROP ND1000 (ThermoFisher Scientific, Waltham MA). The final concentrations were 68 mg/ml and 54 mg/ml for TD01B46 (stapled) and TD01B49 (unstapled) bispecific proteins, respectively. The concentrated samples were split into two equal parts, and one stored at 4° C. and the other stored at 40° C. Aliquots from the incubated samples were taken at different time points (t0, t−1 wk, t−2 wk and t−6 wk) and diluted to 1 mg/mL with 1×dPBS for purity analysis by SEC. For SEC, 20 μg of the 1 mg/mL sample was loaded onto an analytical size exclusion HPLC (TSKgel BioAssist G3SW×1, 7.8 mm ID×30 cm H, 5 μm, TOSOH; guard column: TSKGEL BIOASSIST SW×1 guard column, 6 mm ID×4 cm H, 7 μm, TOSOH; Agilent HPLC system) and monitored for separation of the sample at UV 280 nm, at 1 mL/min for 20 min using 200 mM sodium phosphate (pH 6.8) as the running buffer.

### 6.3.1.15 Cytotoxicity Assay

To determine the effect of BCMA targeting test molecules upon T cell activation and killing potential of tumor cells, the INTELLICYT IQUE3 and FORECYT software (Sartorius) was utilized. H929-Fluc-GFP cells served as target cells for two human donor Pan T cells (Hemacare). The assay was set up in a 96-well plate at a T cell to target ratio of 3:1. Test molecules were added at a starting concentration of 10 nM and serially diluted at 1:4 in complete media. All molecules were tested in duplicate at minimum. Detection of killing and T cell activation status was assessed 72 hrs. later by flow cytometry. Endogenous GFP expressed in H929 was used to separate T cells from target cells. Cytotoxicity was measured using Near-IR Live/Dead stain (ThermoFisher), while activation in CD4 and CD8 T cells was assessed with anti-human CD25-BV650 (BD Biosciences), anti-human CD4-BV510 (Biolegend) and anti-human CD8-PE/Cy7 (Biolegend). Using PRISM software (GRAPHPAD), cytotoxicity and CD25 MFI data was exported, log-transformed, and 4 parameter logistically fit to generate regression curves for reporting of EC50.

### 6.3.1.16 Cell Binding

Briefly, H929 WT and KO cells were counted and stained with CFSE (BD Pharmingen, cat #C34554) and/or CELL TRACE violet proliferation dyes (BD Pharmingen, cat #C34557) as well as near IR live/dead stain (Thermofisher, cat #L10119). Each cell population was quenched with FBS (Gibco, cat #16000-036), Fc-blocked with Human TRUSTAIN FcX blocking reagent (Biolegend, cat #422301) and then plated together in 96 well plates with 50K total cells per well. The cells were then incubated with serial dilutions of test molecules for 1 hr at 37° C. (1/2 log serial dilutions starting at 2 μM). The cells were washed 2× in FACS buffer (Becton Dickinson, cat #554657) and then incubated with 1 μg/mL AF647-labelled goat anti human Fc (Jackson IR, cat #109-606-098) detection reagent for 30 minutes at 4° C. The cells were again washed 2× in FACS buffer and then analyzed on the INTELLICYT IQUE 3 (Sartorius) high throughput flow cytometer. The raw data was exported and analyzed in GRAPHPAD PRISM.

### 6.3.2 Results

6.3.2.1 “Stapling” scFv Concept and Design

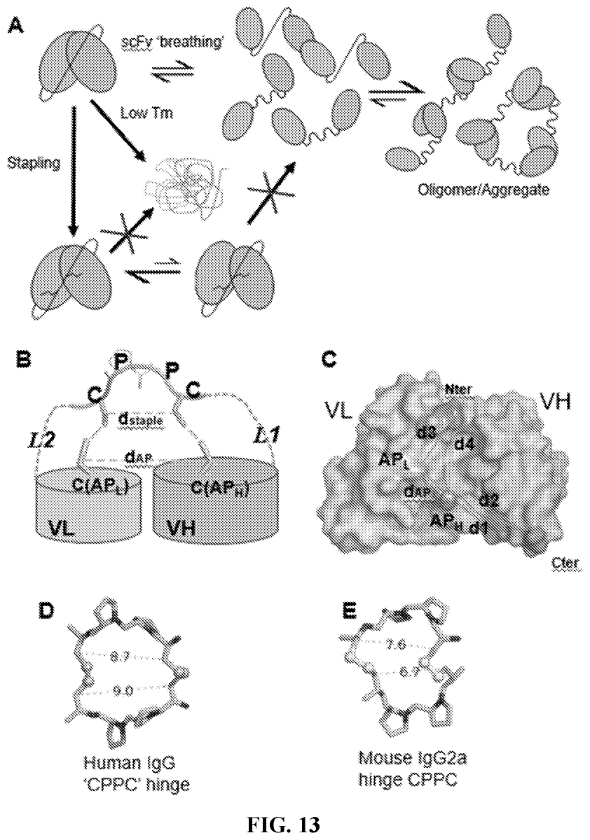

The scFv “stapling” strategy to minimize aggregation due to scFv instability and “breathing” is shown in FIG. 13A. The stapling design is schematically illustrated in FIG. 13B. In this strategy, disulfide bonds (SSs) were attempted to be engineered in between the two Cys residues placed in the flexible linker and the Cys residues (one each) introduced into the VL and VH domains. The scheme of linker-anchor points disulfide bonds mimics the effects of a staple and was thus termed “stapling” (FIG. 13A and FIG. 13B). Without wishing to be bound by theory, it was hypothesized that these SS bonds would restrict their transient and reversible “breathing” and inter-molecular swapping but not negatively impact the small movements between the two variable domains which may be important for antigen binding (Fransson et al. Journal of molecular biology 398, 214-231 (2010)). In addition, stapling would reduce the overall conformational entropy of the stapled scFv (or spFv) as well as that of the flexible linker. This would lead to improvement in spFv stability.

For stapling to correctly form, a number of structural conditions need to be satisfied: (1) the two domain Cys residues (anchor positions) be selected such that the distance (d_{AP}) between them had a narrow distribution but also have a low probability of forming inter-VL/VH disulfide bond directly; (2) the distance between the two linker Cys residues (dₛₜₐₚₗₑ) was compatible with (d_{AP}) and these Cys residues have a low probability of forming a disulfide loop; and (3) the linker Cys residues be positioned close to the respective anchor Cys simultaneously, a condition that was the result of the linker segments L1 (from C terminus of the leading domain to first linker Cys) and L2 (second linker Cys to trailing domain N terminus).

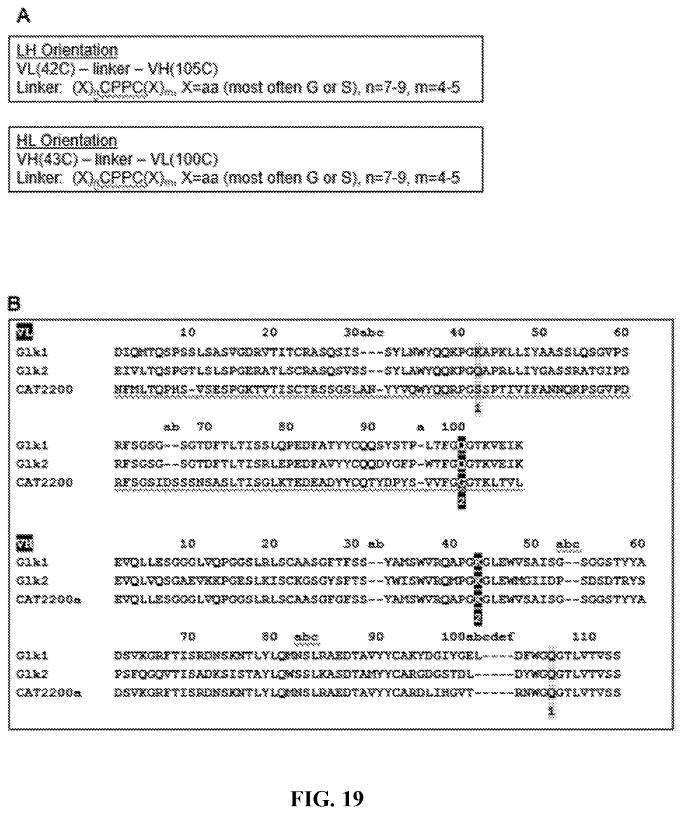

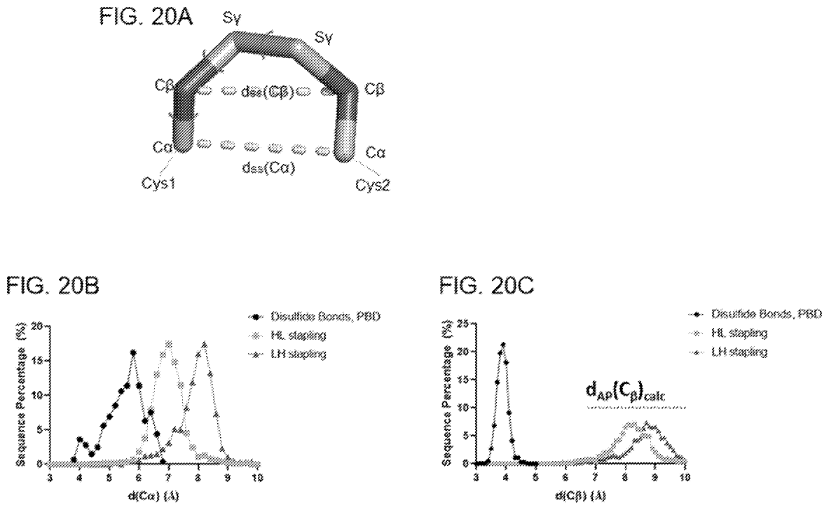

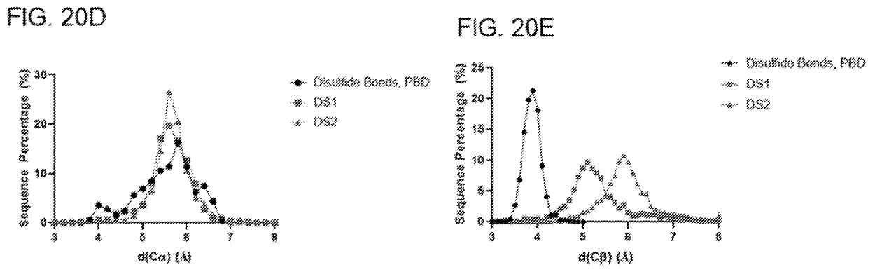

For the “stapling” scheme to be widely applicable, anchor points were selected that were structurally conserved, exposed on surface of both VL and VH and whose mutation to Cys residue would not impact folding of VL and VH or binding to antigens. FIG. 19A illustrated one choice of the positions for the two different orientations of the spFv. For LH orientation, the chosen anchor points were position 42 for VL and position 105 for VH (FIG. 19A) when the sequences were numbered according to the Chothia scheme (Chothia and Lesk, Journal of molecular biology 196, 901-917 (1987)); for HL orientation, the chosen anchor points were Chothia position 43 for VH and position 100 for VL (FIG. 19A). The anchor positions were also illustrated in several antibody sequences (FIG. 19B). The distances and geometry of the anchor positions and domain termini were illustrated in FIG. 13C. The distances between both Cα (d_{AP,Cα}) and Cβ (d_{AP,Cβ}) atoms of these positions were determined from a survey of 2501 Fvs currently in the PDB (data shown in FIGS. 20B-20E). Both distances had a rather narrow and similar distribution in antibody structure. The LH and HL Cα distances had an average of 8.2 Å (range of 7-9 Å) and 6.9 Å (range of 6-8 Å), respectively (FIG. 20B, orange and green curves). The corresponding Cp distances were 8.2 Å (range of 7-10.0 Å) and 8.7 Å (range of 7-10.0 Å), respectively (FIG. 20C, orange and green curves). These distances were much wider than the typical Cα and Cβ distances (4-6.8 Å in FIG. 20B, and 3.5-4.8 Å in FIG. 20C, respectively as shown by black curves) of two positions that form direct disulfide bonds. These distances indicated low probability of the two anchor positions effectively forming disulfide bonds with each other as their distances, on average, were too far from each other. Thus, these two anchor residues were likely available for stapling. Since the d_{AP} distances had a narrow range and were similar in antibodies containing either kappa or lambda light chains due to structural conservation, this set of anchor points likely would be compatible for most Fv molecules.

For the linker, a short sequence of “CPPC” (Cys-Pro-Pro-Cys) was selected as one possible stapling motif because this sequence natively occurs in human IgG1 hinge as well as some rodent IgGs. The structures of the human (Scapin et al. Nat Struct Mol Biol 22, 953-958 (2015)) and mouse IgG (Harris et al. Biochemistry 36, 1581-1597 (1997)) mAb molecules showed that the Cβ(Cys1)-Cβ(Cys2) distances in the human and mouse IgG hinges range from 7 to 9 Å (FIG. 13D and FIG. 13E). This range was comparable to the distances between the two anchor points in both LH and HL orientations (FIG. 13C and FIG. 20C). In addition, the CPPC motif was found to have the slowest rate of SS loop closure (Zhang and Snyder, J Biol Chem 264, 18472-18479 (1989)), due to the relative conformational rigidity of the Pro-Pro residues. Thus, the CPPC staple would likely be of the right geometry for stapling, i.e., forming proper disulfide bonds correctly to the anchor point Cys residues.

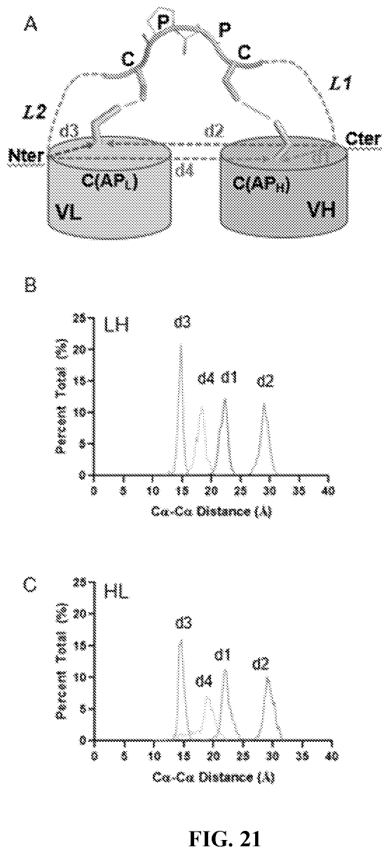

The placement of the stapling motif (CPPC) within the linker was not only important to allow proper stapling disulfide formation as designed, but also to prevent scrambling, i.e., formation of SS between unintended Cys residues. For proper stapling, the first Cys residue must disulfide bond to the anchor point on the leading domain (VL domain in LH or VH in HL) and the second Cys residue to the anchor point on the trailing domain (VH domain in LH or VL in HL). The proper juxtaposition of the CPPC Cys residues to the anchor positions was determined by the length or number of residues of the linker segments L1 and L2, preceding and following the stapling motif, respectively as well as the geometry and distances (d_{AP} and d1-d4) between the anchor points and the termini of the VL and VH domains (FIG. 13C and FIG. 21A). The distance distributions between these positions for the same set of Fvs in the PDB are shown in FIG. 21B (Cα-Cα) and S3C (Cβ-Cβ). Optimal L1 and L2 lengths would bring a linker Cys to within a short distance (Cα atom distance of ˜5-6 Å) of the intended anchor Cys, while keeping the distance between the linker Cys and the opposing anchor Cys residue as distant as possible. For the first stapling disulfide pair, these two distances were determined by L1, d_{AP}, d1 and d2, the distances between the C terminus of the leading domain and the two anchor Cys positions (FIG. 13C and FIG. 21A). As there was a clear difference between d1 and d2 distances, when the linker segment L1 length was optimal, then the first linker Cys and the trailing anchor position Cys on the second domain would be too far apart to form an unwanted SS bond (FIG. 21A). The same was true for the second stapling SS bond, except that linker segment L2, d_{AP} and distances d3 and d4 were the determinants. Molecular modeling suggests that these distances can be spanned by flexible sequences of lengths of 7 to 9 residues long for L1 before the stapling motif and 4 to 5 residues in length for L2 after. These linker lengths were denoted as n+4+m, where n=7-9 and m=4-5 (FIG. 19A). These lengths were predicted to be long enough to allow “stapling” but too short to allow SS scrambling based on distance considerations above. However, the exact L1 and L2 lengths were difficult to predict accurately due to Fv structural variability, the range was sampled experimentally to define the optimal lengths (see below). On the other hand, since the distances and geometry for the chosen set of anchor positions for both the LH and HL orientations were very similar (FIG. 21B and FIG. 21C), it was likely that a linker of equal composition, selected based on modeling and further experimentally validated, would work for both LH and HL constructs. FIG. 19B shows the proposed first set of linker designs with the CPPC staple.

6.3.2.2 Model and Therapeutic spFv Molecules were Significantly More Stable

In order to assess the “stapling” designs, several antibodies were selected to generate scFv and corresponding spFv: two antibodies with kappa light chains (GLk1 and GLk2) from the synthetic phage antibody libraries (Shi et al. Journal of molecular biology 397, 385-396 (2010)) and a lambda-containing antibody (CAT2200) obtained from a publication (Gerhardt et al. Journal of molecular biology 394, 905-921 (2009)) and two scFv variants, Cris7a and Cris7b, derived from anti-CD3 mAb Cris7 (Alberola-IIa et al. J Immunol 146, 1085-1092 (1991)), which have potential for use in CD3-based T-cell redirecting. The anchor points and amino acid sequences of VH and VL domains of several of these antibodies were shown in FIG. 19B. The scFv and spFv molecules in both LH and HL orientations were constructed and expressed. For the scFv constructs, a standard (G₄S)₄ linker was used. For the spFv, different linker lengths within the n and m ranges above were sparsely sampled. These scFv and spFv proteins were expressed and purified from Expi293 cells as described in the Methods. The SDS-PAGE showed that the reduced scFv and spFv migrate identically, however, the non-reduced SDS-PAGE showed spFv molecules migrate faster than the corresponding scFv (results for Cris7a/b scFv/spFv shown FIG. 2B), indicating that additional disulfide bonds in spFvs were formed as expected.

The thermal stability of the scFv and spFv molecules was investigated by differential thermal calorimetry (DSC). The data are shown in Table 12 and Table 14 with select DSC profiles for Cris7a/b shown in FIG. 2A. Comparison of corresponding scFv and spFv proteins indicated a roughly 10° C. increase in Tm upon stapling, regardless of the Tm of the starting scFv (Table 12 and Table 14). For example, the Tm of the Cris7a/b LH scFv's was 59.7 and 57.1° C., respectively (FIG. 2A). The corresponding spFv proteins had Tm of 71.6 and 68.6° C., respectively. This indicated a stabilization (ΔTm) of more than 11° C. (FIG. 2A, Table 12 and Table 14). Glk1 spFv had a Tm of ˜80° C., an increase of 9° C. from its scFv (Table 12 and Table 14). This was comparable to the Tm of its Fab counterpart (Teplyakov et al. mAbs 8, 1045-1063 (2016)). It seemed that for this scFv, stapling nearly restored its thermal stability due to the loss of CH1/CL domains. There was only one exception in the case of GLk2 scFv and HL spFv where the increase in thermal stability was only ˜7° C. This was likely due to the shorter leading segment of the 6+4+6 linker which may have caused slight strain in the stapling geometry. In addition to Tm changes, thermal melting of spFv proteins involved 20% or more enthalpy change (ΔH) (Table 12 and Table 14). On the other hand, the Tm and ΔH values of both LH and HL configurations of the same VL/VH pair were very similar (Table 12 and Table 14), indicating that scFv configuration did not significantly impact thermal stability. The large positive ΔTm and ΔH indicated that the VL and VH domains were apparently stabilized even though there were no mutations in either VH or VL except the anchoring positions which were not expected to impact domain stability. The results also showed that the lengths of the leading and trailing linker segments did not impact the thermal stability improvements (˜11° C. for CAT2200 with 9+4+4, ˜11° C. for Cris7 with 8+4+4, ˜11° C. for Glk2 HL spFv with 9+4+5, and 9° C. with 9+4+5 for Glk1 HL and LH spFv). Overall, these results indicated that leading and trailing linker segment lengths of 8-9 and 4-5 residues, respectively, were compatible with significant stabilization of the stapled scFv in comparison with their scFv counterparts.

To select a stapling linker that was most widely applicable, the impact of linker segment length on the thermal stability of spFv constructs was further explored. With two VH/VL pairs, L1 (7, 8 and 9 residues) and L2 (4, 5 residues) segment lengths were varied in all combinations in LH only. The thermal stability of these scFv/spFv molecules was assessed with fluorescence using UNCLE (Unchain Labs, CA). The Tm data are shown in Table 14. The results indicate that the Tm and ΔTm were essentially identical for constructs with 8 and 9 residue L1. For several spFv proteins with 7 residue L1, the Tm appeared to be slightly lower, suggesting the shorter L1 might be less favorable for thermal stabilization. The trailing segment L2 lengths of 4 and 5 residues did not lead to any significant differences in Tm. Thus, the longer version 9+4+5 was selected for better compatibility with a wide range of scFv molecules.

TABLE 14
Thermal stability of scFv/spFv with different
linkers for two VH/VL pairs.

| Mol Name | Orientation | Linker | Tm | ΔTm |
|---|---|---|---|---|
| MSCW250 | LH | 4x G4S | 55.0 | |
| MSCW258 | LH | 9 + 4 + 5 | 66.4 | 11.4 |
| MSCW259 | LH | 8 + 4 + 5 | 66.8 | 11.8 |
| MSCW260 | LH | 7 + 4 + 5 | 66.1 | 11.1 |
| MSCW261 | LH | 9 + 4 + 4 | 66.8 | 11.8 |
| MSCW262 | LH | 8 + 4 + 4 | 66.2 | 11.2 |
| MSCW263 | LH | 7 + 4 + 4 | 64.8 | 9.8 |
| CE158A | LH | 4x G4S | 55.3 | |
| MSCW270 | LH | 9 + 4 + 5 | 66.6 | 11.3 |
| MSCW271 | LH | 8 + 4 + 5 | 66.6 | 11.3 |
| MSCW272 | LH | 7 + 4 + 5 | 65.7 | 10.3 |
| MSCW273 | LH | 9 + 4 + 4 | 67.1 | 11.7 |
| MSCW274 | LH | 8 + 4 + 4 | 67.2 | 11.8 |
| MSCW275 | LH | 7 + 4 + 4 | 65.1 | 9.8 |

6.3.2.3 Structures of spFv's Reveal Proper “Stapling” and VL/VH Pairing

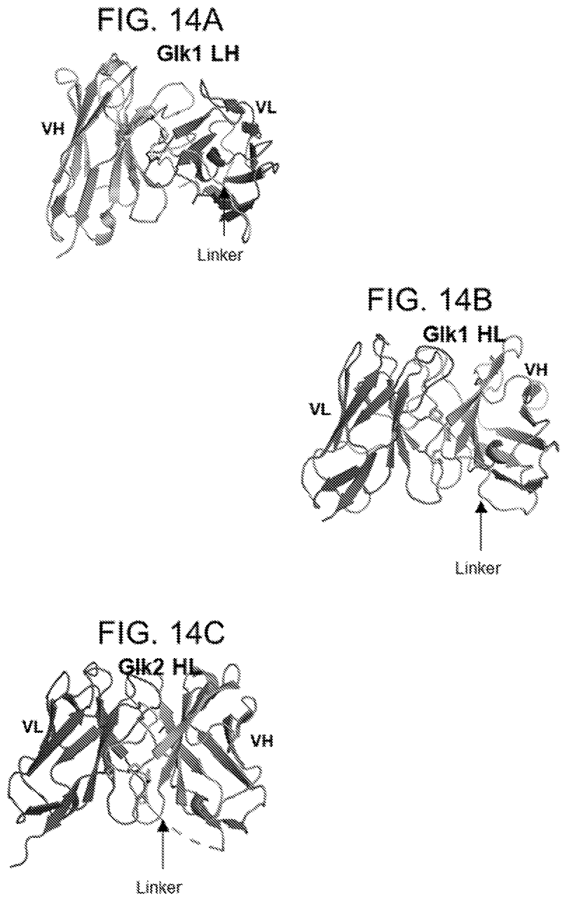

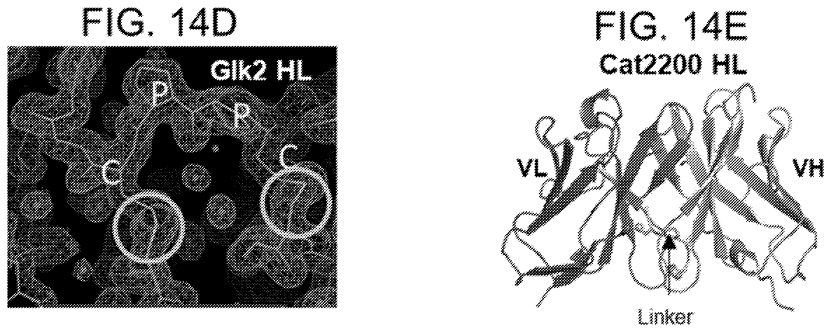

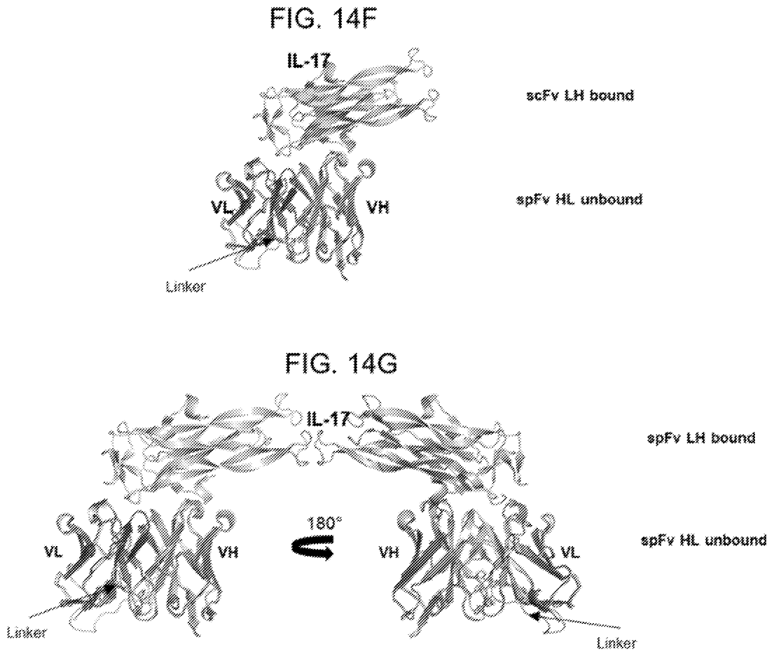

To confirm the proper formation of “stapling disulfide bonds” and to reveal any structural consequences, several scFv and spFv molecules were attempted to be crystallized and structures determined, some in complex with the target protein (Table 13). Interestingly, none of the scFv proteins alone produced any crystals. The overall structures of the unbound spFv and some scFv/spFv:antigen complexes are shown in FIG. 14. The structures were consistent with the typical Fv structures with both VL and VH domains packing against each other. In all spFv structures, some residues of the linker were ordered and resolved in the electron density maps. The disulfide bonds between the “staple” and the anchor points were generally well ordered in both LH and HL orientations (FIGS. 14A-14E). A representative electron density map of the stapling linker region for Glk2 is shown in FIG. 14D. The two stapling SS bonds and linker CPPC motif were well ordered, indicating that stapling indeed occurred as designed. In contrast, no linker residues were ordered in the only scFv containing complex of CAT2200 and IL-17 (FIG. 14F).

For Glk1, structures of LH and HL spFv (FIG. 14A and FIG. 14B) were obtained. There were four independent copies of spFv in the LH crystal and 2 independent copies in the HL crystal. Within LH, the pairwise Cα rmsds including the stapling linkers between spFv copies ranged from 0.22 Å to 0.40 Å. The pairwise Cα rmsd between the two HL spFv molecules was 0.20 Å. When the LH and HL spFv structures were compared, the pairwise Cα rmsds were slightly higher (from 0.53 to 0.77 Å, average 0.61 Å) excluding the linker for 195 to 219 Cα atoms (average 213). The LH and HL spFv was also compared with the Fv fragment in its corresponding Fab (PDB ID 5I19) ((Teplyakov et al. mAbs 8, 1045-1063 (2016))). The rmsds between LH spFv and Fab Fv were from 0.41 to 0.64 Å (average 0.57 Å) for 203 to 215 Cα atoms. The rmsds between HL spFv and Fab Fv were 0.56 to 0.71 Å (average 0.64 Å) for ˜210 Ca atoms. These values indicated that the structures of LH, HL spFv and the Fab Fv were very similar. The small differences were likely the result of crystal packing.

In addition to the unbound spFv structures, the structural impact on antigen binding was attempted to be identified. CAT2200 scFv and spFv molecules were crystallized in complex with its cognate target, IL-17. For the scFv and spFv of CAT2200 variants crystallized, the structures were nearly identical with and without a bound target (FIGS. 14E-14G). They were also identical regardless of orientation or presence or absence of the staple. The rmsd for all matching Cα atoms between pairs of structures were very small: 0.41 Å between unbound spFv-VH-VL and antigen-bound scFv-VL-VH (FIG. 14F), 0.46 Å between unbound spFv-VH-VL and bound spFv-VL-VH (FIG. 14G), and 0.37 Å between bound scFv and bound spFv LH/HL. These structural data showed that stapling did not impact the domain structures of VL and VH or relative VL/VH packing.

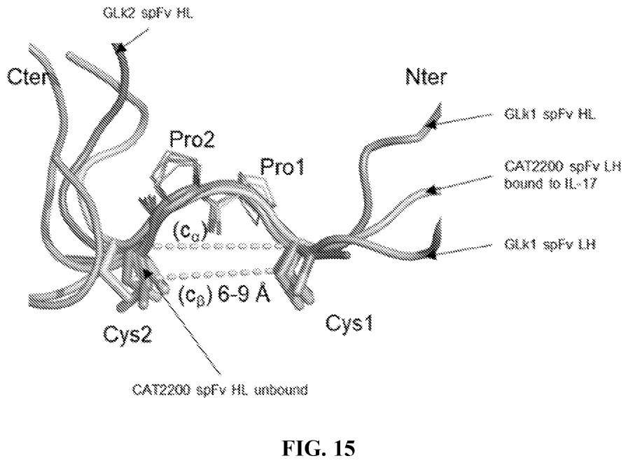

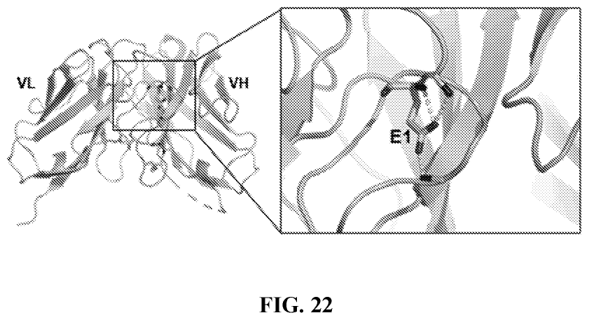

As noted above, the CPPC motif and the disulfides between the staple and anchor points were generally well ordered in the structures. FIG. 15 shows the ordered structures of the linkers superimposed on the CPPC motif. It was clear that the CPPC structures were very similar with main chain atom rmsd of 0.37 Å (range 0.15 to 0.5 Å). The Cβ (Cys1)-Cβ (Cys2) distances ranged from 6-9 Å and Cα (Cys1)-Cα (Cys2) distances were from ˜6.5 Å to 9 Å. These distances were comparable to those in the IgG hinge structures. In the structures the two Pro residues adopted the trans conformation and there was a high degree of similarity between the CPPC motif structures in these different crystals. This was consistent with the notion that Pro-Pro motif was relatively rigid. This rigidity likely applied a force to strengthen the VL and VH domain interactions. At the same time, the observed range of Cp (Cys1)-Cβ (Cys2) distances also indicated that some flexibility was still allowed for proper VL/VH orientation. The linker residues beyond the CPPC motif typically were either disordered or had different conformations (FIG. 15). The leading and trailing linker segments typically did not have specific interactions with the main VL and VH domains except for occasional H bonds between the main chains. For example, in the GLK2 spFv structure, the E1 residue of the trailing VL domain formed sidechain H bond interactions with the trailing linker segment (GLK2 HL spFv, FIG. 22). However, such sidechain interactions between the linker regions and VL/VH domains were likely Fv domain specific and appeared to have little effect on the Fv structures.

6.3.2.4 spFv Bispecifics (BCMA Fab×CD3 scFv/spFv) Show Improved Protein Quality

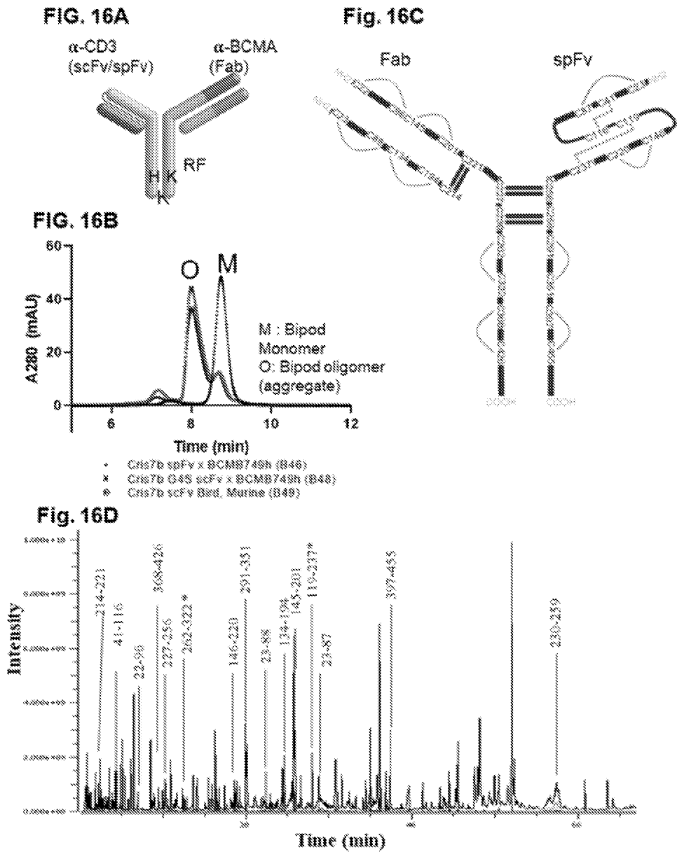

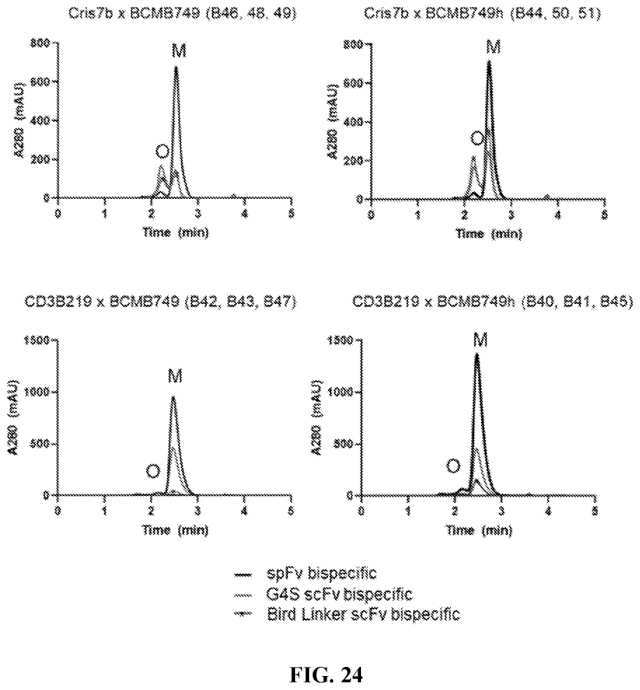

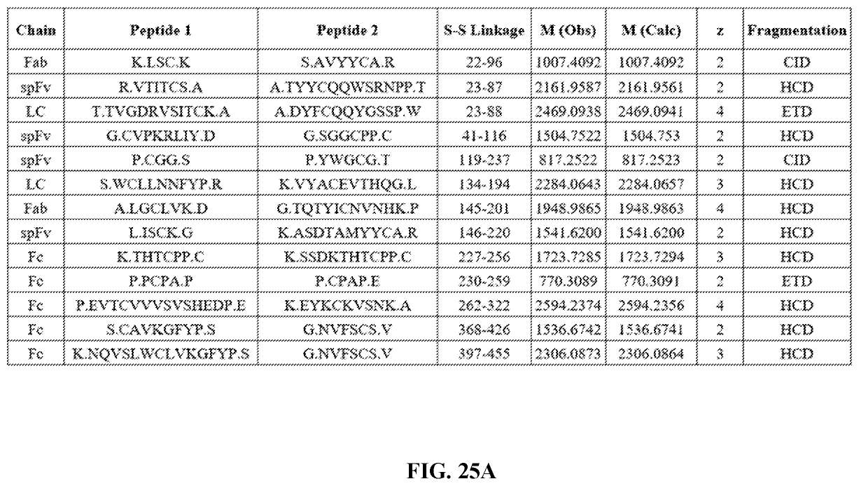

Several sc/spFv×Fab bispecific constructs (illustrated in FIG. 16A) were generated to further study the activity, biophysical properties and translatability of the stapled scFv molecules in a more relevant therapeutic molecular background. Expanding from the prior work with Cris7 domains, two unique anti-CD3 scFv/spFv binding arms were paired, Cris7b as described above and CD3B219, which was derived from SP34 (Pessano et al. EMBO J 4, 337-344 (1985)) and combined them with two related anti-BCMA Fab moieties using the knob-in-hole heterodimerization platform (Ridgway et al. Protein Eng 9, 617-621 (1996)). For the scFv constructs, the Bird linker (Bird et al. Science 242, 423-426 (1988)) was also included to assess impact of linker composition. One anti-BCMA arm, BCMB749, was derived from a mouse monoclonal antibody. The second anti-BCMA arm, BCMB749h, was a humanized variant in which the complimentary-determining regions (CDRs) of BCMB749 were grafted onto a human VL and VH acceptor germline, with a small number of back mutations to the mouse parent sequence (Details in FIG. 23). Pairing the two BCMA Fab arms with two different anti-CD3s each in 3 formats (scFv with Bird linker, scFv with (G₄S)₄ linker and spFv) generated twelve distinct BCMA-targeting bispecific molecules (FIG. 24). The constructs are listed in Table 15.

TABLE 15
Construct design for bispecific proof of concept anti-BCMA targeting antibodies.

| | | anti-CD3 scFv/ | BCMA arm | BCMA arm |
|---|---|---|---|---|
| Name | Molecule Description | spFv arm | HC | LC |
| TD01B49 | BCMB749 × Cris7b scFv Bird | Cris7b scFv Bird | BCMB749 HC | BCMB749 LC |
| TD01B48 | BCMB749 × Cris7b scFv G4S | Cris7b scFv G4S | BCMB749 HC | BCMB749 LC |
| TD01B46 | BCMB749 × Cris7b spFv | Cris7b spFv | BCMB749 HC | BCMB749 LC |
| TD01B43 | BCMB749 × CD3B219a99v scFv Bird | CD3B219a99v scFv Bird | BCMB749 HC | BCMB749 LC |
| TD01B42 | BCMB749 × CD3B219a99v scFv G4S | CD3B219a99v scFv G4S | BCMB749 HC | BCMB749 LC |
| TD01B47 | BCMB749 × CD3B219a99v spFv | CD3B219a99v spFv | BCMB749 HC | BCMB749 LC |
| TD01B51 | BCMB749h × Cris7b scFv Bird | Cris7b scFv Bird | BCMB749H HC | BCMB749H LC |
| TD01B50 | BCMB749h × Cris7b scFv G4S | Cris7b scFv G4S | BCMB749H HC | BCMB749H LC |
| TD01B44 | BCMB749h × Cris7b spFv | Cris7b spFv | BCMB749H HC | BCMB749H LC |
| TD01B41 | BCMB749h × CD3B219a99v scFv Bird | CD3B219a99v scFv Bird | BCMB749H HC | BCMB749H LC |
| TD01B40 | BCMB749h × CD3B219a99v scFv G4S | CD3B219a99v scFv G4S | BCMB749H HC | BCMB749H LC |
| TD01B45 | BCMB749h × CD3B219a99v spFv | CD3B219a99v spFv | BCMB749H HC | BCMB749H LC |

All bispecific samples were expressed from Expi-CHO mammalian cell culture and purified through a two-step process, as described in Methods. Analytical size-exclusion chromatography (aSEC) was performed to assess sample purity post-purification and revealed significant differences between the scFv and spFv-containing bispecific molecules (FIG. 24). In scFv-containing samples, the overall yield was lower for the desired monomer. Additionally, in samples containing Cris7b scFv arm, there was a notable production of higher molecular weight species in the purified samples (FIG. 24, upper plots). In contrast, the spFv containing constructs usually have higher yields and >98% bispecific monomer (FIG. 24). These results demonstrate that stapling in either CD3 scFv arm significantly improved bispecific product yields and quality. The identity of the flexible linker, either (G₄S)₄ or Bird, did not show any appreciable differences.

One set of constructs was chosen to move into larger scale expression and purification to generate large batches of highly purified recombinant antibodies that could be used in a panel of analyses to further test the stapling technology. Based on the data as described above, Cris7b containing molecules were chosen—TD01B46 (BCMB749×Cris7b spFv), TD01B48 (BCMB749×Cris7b scFv G4S) and TD01B49 (BCMB749×Cris7b scFv Bird). Large-scale expression in Expi-CHO and a more thorough purification of these antibody samples revealed more pronounced trends in product quality and yield, as was seen in the small-scale purified samples (FIG. 16B). Production of scFv-containing bispecific samples (B48, B49) showed a large, higher molecular weight oligomeric peak (“O”) as dominating the post-CH1 purified sample, even more pronounced that the corresponding small scale expression (FIG. 24). In comparison, the spFv-containing bispecific (B46) remained a dominant peak that corresponded to the desired monomeric peak. These data together emphasized the improvement shown in protein production and quality when the stapling was applied.

6.3.2.5 Mass Spec Disulfide Mapping Data Confirms the Expected Disulfides in Stapled scFv

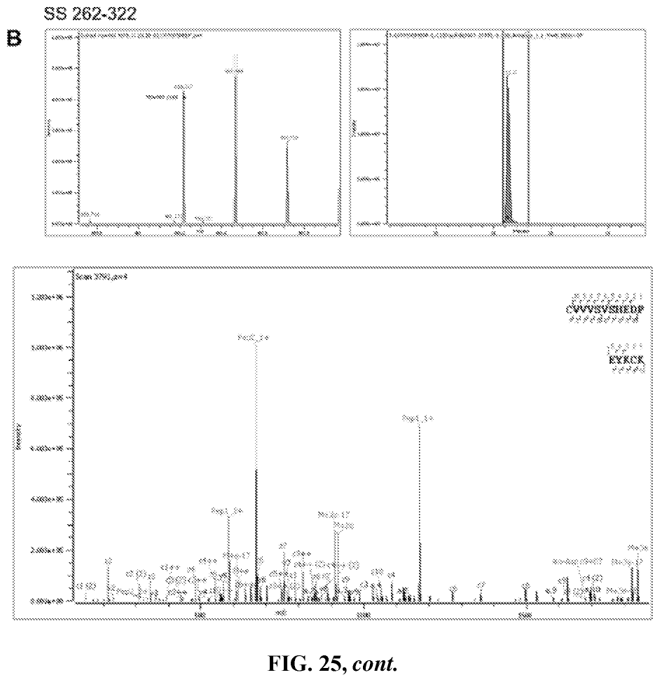

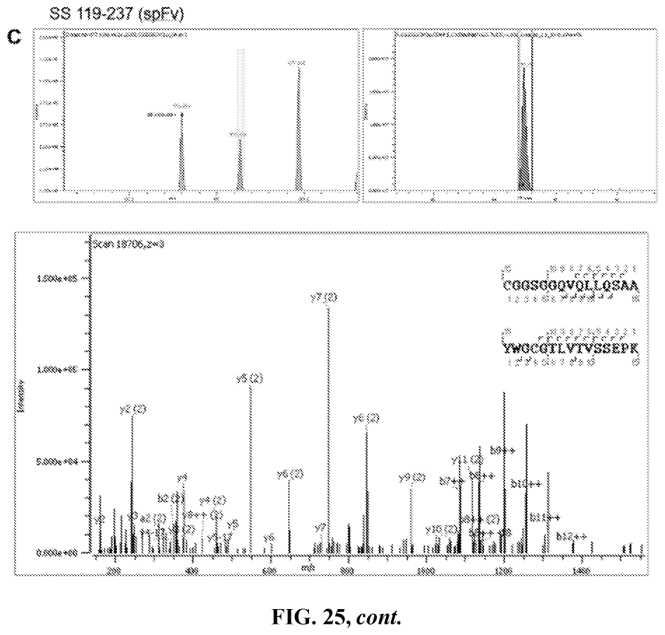

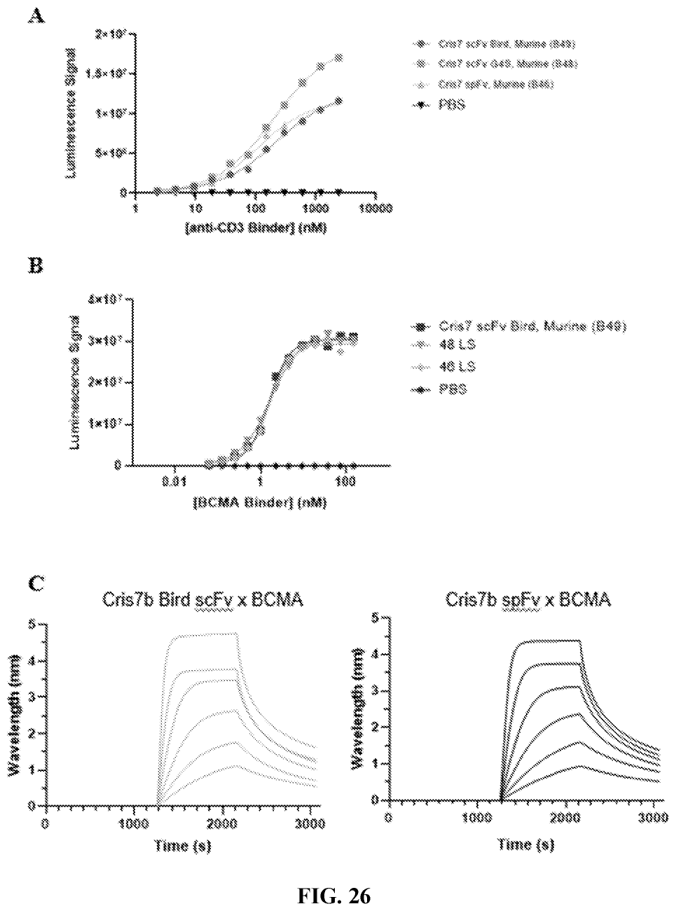

Disulfide mapping LC/MS experiments were used to confirm expected disulfide formation and identify presence of scrambling and free thiol. The stapled bispecific (TD01B46) was digested with a two-enzyme combination as described in the method section and expected disulfide links were determined. FIG. 16C shows all the expected disulfide links. A total ion chromatogram of the digested protein is shown in FIG. 16D. The peaks corresponding to the expected peptides connected by disulfides were identified based on peak intensity in the extracted ion chromatogram (XIC), mass of the disulfide complex (MS1) and peptide sequences (MS2). Representative XIC, MS1 and MS2 data for several of the disulfide peptide complexes are shown in FIG. 25 and FIG. 26. Complete coverage of every expected disulfide was confirmed by this LC/MS/MS approach and the expected disulfides represented >99% of all cysteine containing peptides detected. In this study, SS scrambling was also observed at <0.5%, which was lower than the level of a typical mAb. These data confirm that all expected SS bonds, including the stapling ones, in FIG. 16C and FIG. 16D were correctly formed.

### 6.3.2.6 Stapled Bispecifics Show Improved Thermal Stability

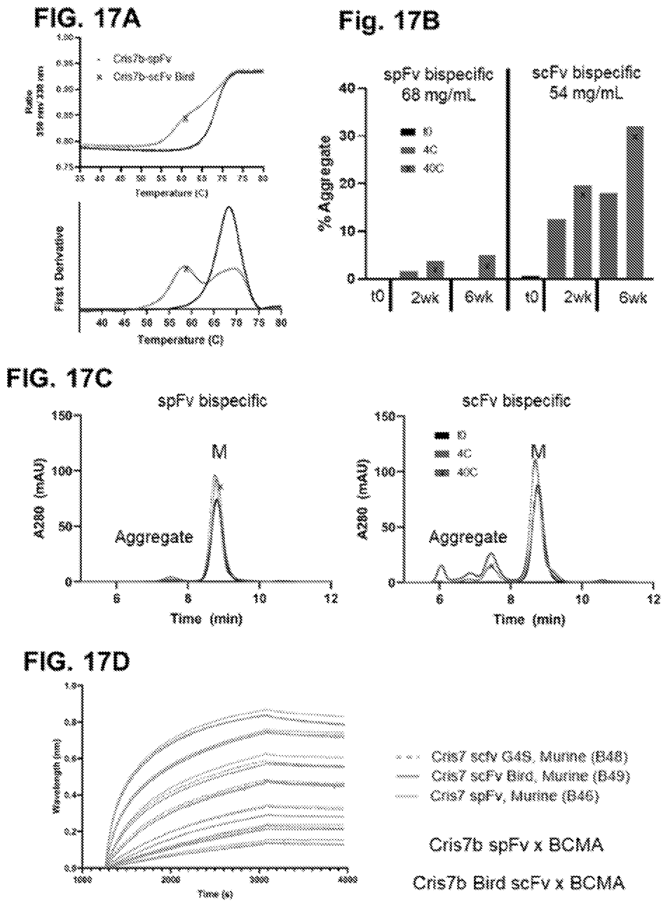

Next, the impact of stapling on the thermal stability on the bispecific constructs was evaluated using NanoDSF measurements (FIG. 17A, Table 16).

TABLE 16
Thermal and colloidal stability of stapled bispecifics (LS: large scale).

| | | | σ, | | σ, | | σ, | | σ, |
|---|---|---|---|---|---|---|---|---|---|
| Name | Description | Tonset | Tonset | Tm1 | Tm1 | Tm2 | Tm2 | Tagg | Tagg |
| TD01B49 | BCMB749 × Cris7b scFv | 51.95 | 0.18 | 58.6 | 0.02 | 68.66 | 0.03 | 70.32 | 0.04 |
| | Bird, LS Exp | | | | | | | | |
| TD01B48 | BCMB749 × Cris7b scFv | 51.29 | 0.08 | 59.4 | 0.02 | 68.65 | 0.03 | 70.23 | |
| | G4S | | | | | | | | |
| TD01B46 | BCMB749 × Cris7b spFv, | 61.04 | 0.22 | 68.28 | 0.03 | — | — | 70.51 | 0.18 |
| | LS Exp | | | | | | | | |
| TD01B51 | BCMB749h × Cris7b scFv | 50.98 | 0.23 | 59.27 | 0.07 | 69.76 | 0.09 | 70.8 | 0.16 |
| | Bird | | | | | | | | |
| TD01B50 | BCMB749h × Cris7b scFv | 50.42 | 0.39 | 59.17 | 0.03 | 69.73 | 0.07 | 70.68 | 0 |
| | G4S | | | | | | | | |
| TD01B44 | BCMB749h × Cris7b spFv | 58.38 | 0.08 | 68.54 | 0.04 | — | — | 72.33 | 0.55 |
| TD01B40 | BCMB749h × | 56.19 | 0.2 | 60.55 | 0.04 | 70.83 | 0.05 | 70.39 | 0.23 |
| | CD3B219a99v scFv G4S | | | | | | | | |
| TD01B45 | BCMB749h × | 58.98 | 0.16 | 68.36 | 0.01 | — | — | 72.45 | 0.22 |
| | CD3B219a99v spFv | | | | | | | | |

For Cris7b highly purified samples, the scFv containing construct showed a melting transition at a Tm of ˜59.0° C. (FIG. 17A, Table 16) with Tonset from 50-56° C. The scFv Tm was similar to that of the simple scFv (Table 12 and Table 14). For the corresponding spFv containing bispecifics (FIG. 17A, Table 16), this low Tm transition disappeared, and the first transition now had a Tm of ˜68.3° C., which was apparently convoluted with the transitions of the other domains of the bispecifics. Moreover, the Tonset of this spFv bispecific protein increased to ˜61° C. (FIG. 17A, Table 16). Together, these findings were interpreted to indicate an approximate 10° C. or higher stability improvement for the spFv-containing moiety. Similar improvements in stability were also true for the CD3B219 spFv constructs (Table 16). These data indicate that the thermal stabilization seen in scFv/spFv only constructs (Table 12 and Table 14) was also true when they were incorporated into therapeutic constructs.

6.3.2.7 Stapled Bispecifics were Resistant to Heat Stress Induced Aggregation

The impact of spFv on aggregation induced by heat stress was further evaluated, as a predictive indicator of protein shelf stability at 4° C., an assay widely used (Bailly et al. mAbs 12, 1743053 (2020)). The highly purified Cris7b scFv/spFv bispecifics (TD01B49 and TD01B46, respectively) (purity >98.5%) were concentrated in DPBS to ˜60 mg/ml. The concentrated samples were then incubated at 4 and 40° C. At two-week intervals, a small aliquot of each sample was diluted to 1 mg/ml and then run on aSEC. The results are shown in FIG. 17B and FIG. 17C. Over a six-week incubation period at 4° C., the spFv bispecific TD01B46 (FIG. 17B and FIG. 17C, left panels) remained a monomer, while at 40° C. it showed a modest increase to about 5% dimer species at 6 weeks. By contrast, over the same period at either 4° C. or 40° C., the scFv bispecific TD01B49 (FIG. 17C, right panels) showed a large increase to about 18% and 32% aggregate species (dimers and higher order oligomers), respectively. These data demonstrate that spFv containing bispecifics were much more resistant to heat induced aggregation at high protein concentration.

### 6.3.2.8 Stapled Bispecifics Retain Binding Affinities

To determine whether the stapling impacts binding to target antigens of interest on either the scFv or Fab containing arms, binding was performed using bio-layer interferometry (BLI) and ELISA with highly purified bispecific samples (TD01B46 (BCMB749×Cris7b spFv), TD01B48 (BCMB749×Cris7b scFv G4S) and TD01B49 (BCMB749×Cris7b scFv Bird)). Recombinant CD3E/D heterodimer protein was used to assess binding of the scFv/spFv anti-CD3 arms. Binding measurements showed that the scFv and spFv bispecifics bound CD3 similarly, indicating incorporation of ‘stapling’ to the scFv did not alter CD3 binding (FIG. 17D; FIG. 26A). The sensograms from BLI (FIG. 17D) showed similar binding responses, association and dissociation profiles indicating scFv and spFv bispecifics have similar binding kinetics for CD3. These results were consistent with the ELISA binding data (FIG. 26A). The scFv linker (Bird and G4S) also had no significant impact on affinity for the target protein. The anti-Fab BCMA arm, identical in either the scFv or spFv bispecific molecules, also showed similar binding affinity to recombinant BCMA (FIG. 26B; FIG. 26C).

TABLE 17
BCMA binding of scFv and spFv bispecific molecules by BLI, using 1:1 fitting model.
Both scFv and spFv bispecifics contain the same anti-BCMA murine Fab arm.

| Sample Name | KD (M) | KD Error | ka (1/Ms) | ka Error | kdis (1/s) | kdis Error |
|---|---|---|---|---|---|---|
| TD01B49 | 4.02E−09 | 4.13E−11 | 3.62E+05 | 3.47E+03 | 1.45E−03 | 5.39E−06 |
| TD01B46 | 5.43E−09 | 5.85E−11 | 2.61E+05 | 2.61E+03 | 1.42E−03 | 5.59E−06 |

Together, these data indicate that the spFv retains binding affinity of the corresponding scFv proteins. Incorporation of the stapling mutations also did not impact binding of partner domain in the bispecific molecules of interest.

### 6.3.2.9 Stapled Bispecifics Show Potent Killing in CD3 Target Assays

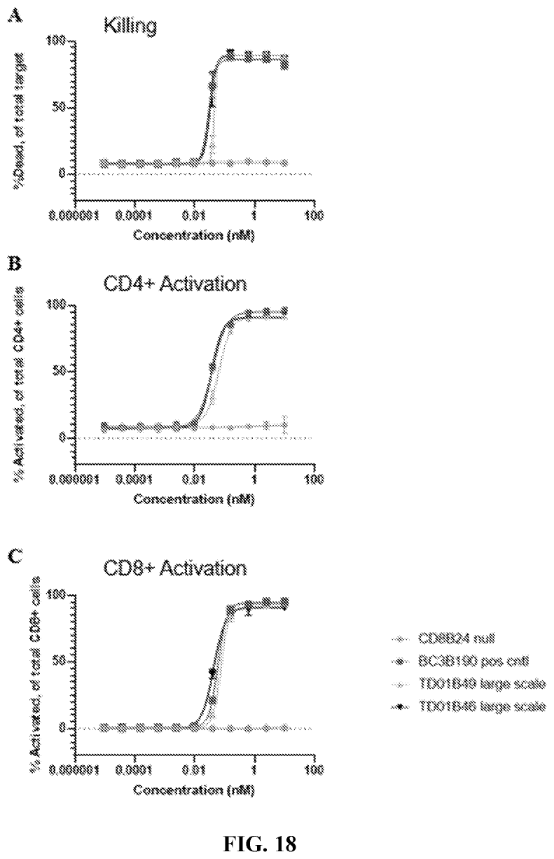

To determine the effect of BCMA targeting bispecific molecules upon T cell activation and killing potential of tumor cells, H929-Fluc-GFP cells served as target cells for two human donor pan-T cells. Detection of killing and T cell activation status was assessed 72 h later by flow cytometry. All bispecific proteins potently killed BCMA+H929−GFP+ cells in a cytotoxicity assay with very similar EC50 (FIG. 18A), whereas a negative control bispecific with a Cris7b scFv/non-targeting Fab (CD8B24) showed no killing activity. All bispecific constructs containing either an scFv or spFv domain also activated CD4+ and CD8+ T cells with similar EC50s whereas the negative control molecule did not activate T cells (FIG. 18B and FIG. 18C). This indicated that the H929 cell killing was the result of T cell activation by CD3 redirecting. Taken together, spFv fully retains the scFv function in therapeutic constructs.

### 6.3.2.10 Stapling Significantly Improved Biophysical Properties of Potential Therapeutic Multispecific Molecules

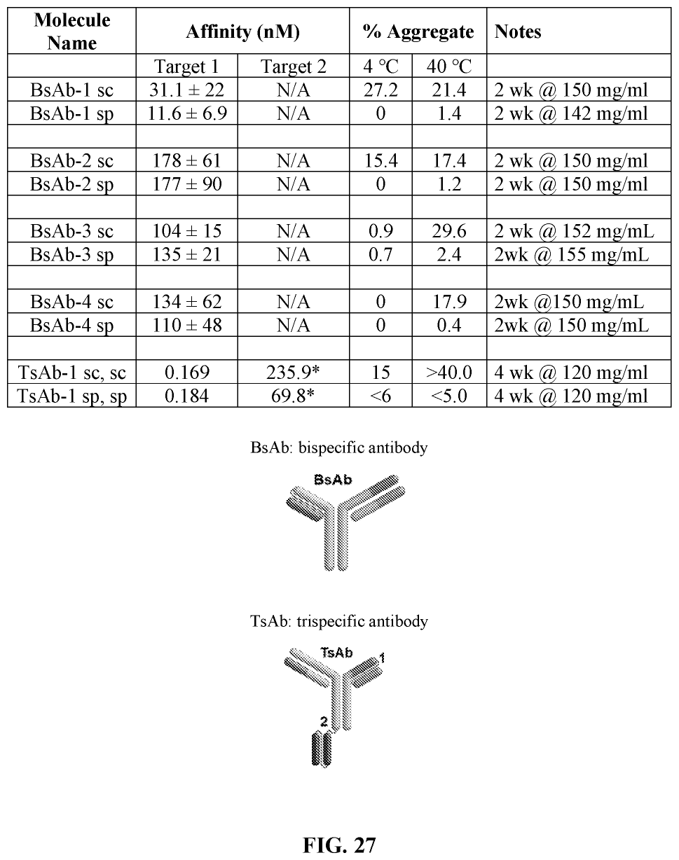

Stapling was applied to a number of proprietary scFv-containing bi- and tri-specific therapeutic antibodies that previously showed poor biophysical properties. While the scFv containing molecules displayed obvious aggregation upon heat stress at high concentrations, the spFv containing counterparts showed much less aggregation under similar conditions (FIG. 27). These molecules still maintained respective target binding. In some cases, antigen binding was slightly improved (FIG. 27). The improvement in biophysical property was very beneficial for downstream therapeutic development that may also lead to improved product quality and therapeutic outcomes.

### 6.3.3 Discussion

In this work, “stapling” was designed to significantly improve scFv stability and reduce its tendency to aggregate. It was achieved by forming specific disulfide bonds between the otherwise flexible linker and two conserved anchor positions on the VL and VH domains. Disulfide bonds in proteins contribute to their stability. In fact, each of VL and VH domains contains a conserved intra-domain disulfide bond that confers considerable stability to the domains. Disulfides increase protein stability by covalently linking distant structural elements together to increase the compactness of protein structure (Geckos et al. Biochemistry 42, 13746-13753 (2003); Betz et al., Protein Sci 2, 1551-1558 (1993)). Stapling as designed improved scFv stability and aggregation by several mechanisms. First, the linker between the VL and VH domains in spFv molecules was generally shown to be ordered, particularly the stapling elements. This significantly reduced the linker conformational entropy. Second, there was a short distance between the two stapling disulfide bonds (about 6-9 Å in observed spFv structures). As a result, the two folded VL and VH domains cannot separate from each other freely, thus reducing the overall conformational entropy of the spFv compared with scFv. Interestingly, the structures indicated that there was very little direct interaction between the ordered linkers and VL and VH domains except for the two stapling disulfide bonds. Stapling also did not impact the relative orientation of the two VL/VH domains. Thus, stapling did not provide new interactions between VH and VL, rather effectively increased existing interactions. Both entropy reduction and increased tethering contribute to the significant increase in Tm of spFv, which in turn reduced domain unfolding, a common factor in scFv protein aggregation. Third, the short distance between the stapling disulfide bonds also prevented VL/VH breathing and inter-molecular swapping, another important factor contributing to scFv aggregation. In contrast, the long linker (typically 15 aa or longer) between the VL and VH domains in an scFv did not provide such restraints. Thus, stapling was an effective scheme to improve scFv stability and reduce aggregation, which are very important factors for developability of scFv-containing biotherapeutics.

The anchor positions selected for stapling were structurally well conserved in all Fv domains of either kappa or lambda light chains. The geometry of the two sets of anchor positions for LH and HL spFv had a relatively narrow range of variation (FIG. 20B and FIG. 20C). Thus, stapling by a simple motif like CPPC was likely applicable to nearly all Fv fragments. This scheme was superior to a previous disulfide mediated scFv stabilization approach, i.e., forming direct disulfide bonds between VL and VH domains (L43-H105, DS1 and L100-H44, DS2). The stabilizing effects of DS1 and DS2 have been inconsistent (Weatherill et al. Protein Eng Des Sel 25, 321-329 (2012)) and as such they have not been widely applied in current therapeutics incorporating scFv moieties. Formation of a disulfide bond between two positions in proteins requires a relatively narrow range of geometry (Dani et al. Protein Eng 16, 187-193 (2003)). The Cα-Cα distance range was 4.8-6.8 Å (peak at 5.6 Å) and Cβ-Cβ distances were in the range of ˜3.4-4.8 Å (peak at ˜3.8 Å) from analyses of protein structures (Dani et al. Protein Eng 16, 187-193 (2003)) (FIG. 20D and FIG. 20E). These parameters, along with environments of the two positions, determine the allowed SS bond conformations which were critical for successful formation. Analysis of 2501 high resolution Fab/scFv structures showed that most Fv structures did not have the geometry compatible with SS bond formation. For example, the Cβ-Cβ distances were distributed around 5.2 Å and 5.8 Å for DS1 and DS2, respectively. These were much wider than those in typical SS bonds in proteins. While protein structural flexibility may allow a subset of structures to form SS bonds, it was also likely that a disulfide between these positions with unfavorable geometry may not form or place a structural strain when formed in a large proportion of Fv domains. On the other hand, the two legs of the stapling motif were external to the Fv domains and can easily position properly to satisfy the linker-anchor point disulfide geometry. Our analysis showed that the distances between the two stapling legs were comparable to the distances of the two sets of anchor points for LH and HL (FIG. 20B and FIG. 20C). Thus, as shown in the multiple examples reported in this work, stapling is much more widely applicable.

In summary, a simple but widely applicable strategy of “stapling” scFv was presented to enhance stability and reduce scFv mediated aggregation. With improved stability over and structural identity to scFv, spFv can be used anywhere a corresponding scFv was used, including bi-, multispecifics, CAR-Ts and other molecular architectures. Stapling improvs successful conversion rate of Fab/mAb to scFv so that more spFv moieties were available for construction of fit-for-purpose molecular entities. The improved biophysical properties resulting from the use of spFv instead of scFv allows construction of biotherapeutics of superior developability, thus enabling faster development and improving drug quality, efficacy and safety.

Lengthy table referenced here
US20250320304A1-20251016-T00001
Please refer to the end of the specification for access instructions.

Lengthy table referenced here
US20250320304A1-20251016-T00002
Please refer to the end of the specification for access instructions.

Lengthy table referenced here
US20250320304A1-20251016-T00003
Please refer to the end of the specification for access instructions.

Lengthy table referenced here
US20250320304A1-20251016-T00004
Please refer to the end of the specification for access instructions.

Lengthy table referenced here
US20250320304A1-20251016-T00005
Please refer to the end of the specification for access instructions.

Lengthy table referenced here
US20250320304A1-20251016-T00006
Please refer to the end of the specification for access instructions.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

LENGTHY TABLES
The patent application contains a lengthy table section. A copy of the table is available in electronic form from the USPTO web site (https://seqdata.uspto.gov/?pageRequest=docDetail&DocID=US20250320304A1). An electronic copy of the table will also be available from the USPTO upon request and payment of the fee set forth in 37 CFR 1.19(b)(3).

1. A molecule comprising an antigen-binding fragment (Fab), a single chain variable fragment (scFv), and a fragment crystallizable region (Fc region), wherein the scFv comprises a heavy chain variable region (VH), a linker (L), and a light chain variable region (VL), wherein the scFv comprises:
a) a disulfide bond between a structurally conserved surface exposed VH position which is mutated to cysteine (Cys) and a L Cys;
b) a disulfide bond between a structurally conserved surface exposed VL position which is mutated to Cys and a L Cys; or
c) a first disulfide bond between a structurally conserved surface exposed VH Cys and a first L Cys, and a second disulfide bond between a structurally conserved surface exposed VL Cys and a second L Cys; and wherein:
the molecule has improved stability, expression yields, and/or quality as compared to a comparable a molecule absent a disulfide bond; and
wherein:
a) the VH comprises a VH Cys at a structurally conserved surface exposed VH framework residue position and the L comprises a L Cys;
b) the VL comprises a VL Cys at a structurally conserved surface exposed VL framework residue position and the L comprises a L Cys; or
c) the VH comprises a VH Cys at a structurally conserved surface exposed VH framework residue position, the VL comprises a VL Cys at a structurally conserved surface exposed VL framework residue position, and the L comprises a first L Cys and a second L Cys, wherein the VH Cys and the first L Cys are capable of forming a disulfide bond, and the VL Cys and the second L Cys are capable of forming a disulfide bond.

2. (canceled)

3. A molecule comprising an Fab that binds to a first antigen, and a scFv that binds to a second antigen, and a Fc region, wherein the scFv comprises a means for stabilizing the scFv; and
wherein:
the scFv comprises a VH, a L, and a VL and wherein the means for stabilizing the scFv comprises:
a) a disulfide bond between a structurally conserved surface exposed VH Cys and a L Cys;
b) a disulfide bond between a structurally conserved surface exposed VL Cys and a L Cys; or
c) a first disulfide bond between a structurally conserved surface exposed VH Cys and a first L Cys, and a second disulfide bond between a structurally conserved surface exposed VL Cys and a second L Cys.

4. (canceled)

5. (canceled)

6. The molecule of claim 1, wherein the distance between the VH Cys and the VL Cys is from about 5 Å to about 10 Å or from about 7 Å to about 9 Å.

7. The molecule of claim 1, wherein
a) the VH Cys is at H3, H5, H40, H43, H46 or H105; and/or
b) the VL Cys is at L3, L5, L39, L42, L43, L45, L100 or L102,
wherein the residue numbering is according to Chothia, and
wherein:
the VH Cys is at H105 and the VL Cys is at L42;
b) the VH Cys is at H43 and the VL Cys is at L100;
c) the VH Cys is at H3 and the VL Cys is at L3;
d) the VH Cys is at H3 and the VL Cys is at L5;
e) the VH Cys is at H3 and the VL Cys is at L39;
f) the VH Cys is at H3 and the VL Cys is at L42;
g) the VH Cys is at H3 and the VL Cys is at L45;
h) the VH Cys is at H3 and the VL Cys is at L100;
i) the VH Cys is at H3 and the VL Cys is at L102;
j) the VH Cys is at H5 and the VL Cys is at L3;
k) the VH Cys is at H5 and the VL Cys is at L5;
l) the VH Cys is at H5 and the VL Cys is at L39;
m) the VH Cys is at H5 and the VL Cys is at L42;
n) the VH Cys is at H5 and the VL Cys is at L45;
o) the VH Cys is at H5 and the VL Cys is at L100;
p) the VH Cys is at H5 and the VL Cys is at L102;
q) the VH Cys is at H40 and the VL Cys is at L3;
r) the VH Cys is at H40 and the VL Cys is at L5;
s) the VH Cys is at H40 and the VL Cys is at L39;
t) the VH Cys is at H40 and the VL Cys is at L42;
u) the VH Cys is at H40 and the VL Cys is at L45;
v) the VH Cys is at H40 and the VL Cys is at L100;
w) the VH Cys is at H40 and the VL Cys is at L102;
x) the VH Cys is at H43 and the VL Cys is at L3;
y) the VH Cys is at H43 and the VL Cys is at L5;
z) the VH Cys is at H43 and the VL Cys is at L39;
aa) the VH Cys is at H43 and the VL Cys is at L42;
bb) the VH Cys is at H43 and the VL Cys is at L45;
cc) the VH Cys is at H43 and the VL Cys is at L102;
dd) the VH Cys is at H46 and the VL Cys is at L3;
ee) the VH Cys is at H46 and the VL Cys is at L5;
ff) the VH Cys is at H46 and the VL Cys is at L39;
gg) the VH Cys is at H46 and the VL Cys is at L42;
hh) the VH Cys is at H46 and the VL Cys is at L45;
ii) the VH Cys is at H46 and the VL Cys is at L100;
ji) the VH Cys is at H46 and the VL Cys is at L102;
kk) the VH Cys is at H105 and the VL Cys is at L3;
ll) the VH Cys is at H105 and the VL Cys is at L5;
mm) the VH Cys is at H105 and the VL Cys is at L39;
nn) the VH Cys is at H105 and the VL Cys is at L45;
oo) the VH Cys is at H105 and the VL Cys is at L100;
pp) the VH Cys is at H105 and the VL Cys is at L102, or
qq) the VH Cys is at H105 and the VL Cys is at L43,
wherein the residue numbering is according to Chothia.

8. (canceled)

9. The molecule of claim 1, wherein the L comprises a contiguous amino acid sequence derived from an immunoglobulin (Ig) hinge region;
optionally wherein the Ig hinge region is derived from a human Ig hinge region or a non-human Ig hinge region, optionally wherein the Ig hinge region is derived from a human Ig hinge region;
optionally wherein the human Ig hinge region is an IgG1, IgG2, IgG3, or IgG4 isotype; and
wherein the L comprises:
a) an amino acid sequence C(X)yC (SEQ ID NO: 23), wherein X is glycine (Gly), serine (Ser), proline (Pro), alanine (Ala), arginine (Arg), asparagine (Asn), aspartic acid (Asp), glutamic acid (Glu), glutamine (Gln), histidine (His), isoleucine (Ile), leucine (Leu), lysine (Lys), phenylalanine (Phe), threonine (Thr), tryptophan (Trp) or tyrosine (Tyr), and y is an integer from 1 to 3,
b) an amino acid sequence C(X)yC (SEQ ID NO: 24), wherein X is Gly, Ser or Pro, and y is an integer from 1 to 3,
optionally wherein the L comprises the amino acid sequence CPC, CGC, CSC, CPPC (SEQ ID NO: 1), CGPC (SEQ ID NO: 28), CPGC (SEQ ID NO: 29), CGGC (SEQ ID NO: 30), CSPG (SEQ ID NO: 31), CPSC (SEQ ID NO: 32), CSSC (SEQ ID NO: 33), CGSC (SEQ ID NO: 34), CSGC (SEQ ID NO: 35), CPPPC (SEQ ID NO: 36), CGPPC (SEQ ID NO: 37), CPGPC (SEQ ID NO: 38), CPPGC (SEQ ID NO: 39), CGGPC (SEQ ID NO: 40), CPGGC (SEQ ID NO: 41), CGGGC (SEQ ID NO: 42), CSPPC (SEQ ID NO: 43), CPSPC (SEQ ID NO: 44), CPPSC (SEQ ID NO: 45), CSSPC (SEQ ID NO: 46), CPSSC (SEQ ID NO: 47), CSSSC (SEQ ID NO: 48), CGSPC (SEQ ID NO: 49), CPGSC (SEQ ID NO: 50), CSGPC (SEQ ID NO: 51) or CPSGC (SEQ ID NO: 52);
c) an amino acid sequence (X)mC(X)yC(X)n (SEQ ID NO: 25); wherein X is Gly, Ser, Pro, Ala, Arg, Asn, Asp, Glu, Gln, His, Ile, leu, Lys, Phe, Thr, Trp or Tyr, m is an integer from 6 to 9, y is an integer from 1 to 3 and n is an integer from 4 to 6;
d) an amino acid sequence (X)mC(X)yC(X)n (SEQ ID NO: 26); wherein X is Gly, Ser, Pro, Ala, Arg, Asn, Asp, Glu, Gln, His, Ile, Leu, Lys, Thr or Tyr, m is an integer from 6 to 9, y is an integer from 1 to 3 and n is an integer from 4 to 6; or
e) an amino acid sequence (X)mC(X)yC(X)n (SEQ ID NO: 27); wherein X is Gly or Pro, m is an integer from 6 to 9, y is an integer from 1 to 3 and n is an integer from 4 to 6.

10. (canceled)

11. The molecule of claim 1, wherein the L has a length of from about 14 to about 19 amino acids, optionally wherein the L has a length of about 14, about 15, about 16, about 17, about 18, or about 19 amino acids.

12. The molecule of claim 1, wherein the L comprises the amino acid sequence of SEQ ID NO: 3, SEQ ID NO: 4, SEQ ID NO: 5, SEQ ID NO: 6, or SEQ ID NO: 7.

13. The molecule of claim 1, wherein the scFv is in the VL-L-VH orientation or wherein the scFv is in the VH-L-VL orientation.

14. (canceled)

15. The molecule of claim 1, wherein
(i) (a) the VH comprises a Cys at H105; (b) the VL comprises a Cys at L42; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(ii) (a) the VH comprises a Cys at H105; (b) the VL comprises a Cys at L45; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(iii) (a) the VH comprises a Cys at H105; (b) the VL comprises a Cys at L39; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(iv) (a) the VH comprises a Cys at H5; (b) the VL comprises a Cys at L42; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(v) (a) the VH comprises a Cys at H5; (b) the VL comprises a Cys at L45; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(vi) (a) the VH comprises a Cys at H5; (b) the VL comprises a Cys at L39; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(vii) (a) the VH comprises a Cys at H3; (b) the VL comprises a Cys at L42; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation;
(viii) (a) the VH comprises a Cys at H3; (b) the VL comprises a Cys at L45; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation; or
(ix) (a) the VH comprises a Cys at H3; (b) the VL comprises a Cys at L39; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VL-L-VH orientation, or
wherein:
(i) (a) the VH comprises a Cys at H43; (b) the VL comprises a Cys at L100; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(ii) (a) the VH comprises a Cys at H43; (b) the VL comprises a Cys at L102; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(iii) (a) the VH comprises a Cys at H43; (b) the VL comprises a Cys at L5; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(iv) (a) the VH comprises a Cys at H43; (b) nthe VL comprises a Cys at L3; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(v) (a) the VH comprises a Cys at H40; (b) the VL comprises a Cys at L100; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(vi) (a) the VH comprises a Cys at H40; (b) the VL comprises a Cys at L102; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(vii) (a) the VH comprises a Cys at H40; (b) the VL comprises a Cys at L5; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(viii) (a) the VH comprises a Cys at H40; (b) the VL comprises a Cys at L3; 9c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(ix) (a) the VH comprises a Cys at H46; (b) the VL comprises a Cys at L100; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(x) (a) the VH comprises a Cys at H46; (b) the VL comprises a Cys at L102; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation;
(xi) (a) the VH comprises a Cys at H46; (b) the VL comprises a Cys at L5; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation; or
(xii) (a) the VH comprises a Cys at H46; (b) the VL comprises a Cys at L3; (c) the L comprises the amino acid sequence of SEQ ID NO: 2, SEQ ID NO: 3, SEQ ID NO: 4, SEQ ID NO: 5, SEQ ID NO: 6, or SEQ ID NO: 7; and (d) the scFv is in the VH-L-VL orientation.

16. (canceled)

17. The molecule of claim 15, wherein the L comprises the amino acid sequence of SEQ ID NO: 3, SEQ ID NO: 6, or SEQ ID NO: 7.

18. The molecule of claim 1, wherein the binding molecules comprises a heavy chain, a light chain, and a polypeptide, wherein the N-terminus of the heavy chain and the light chain form the Fab; wherein the polypeptide comprises the scFv at the N-terminus; and wherein the C-terminus of the polypeptide and the C-terminus of the heavy chain form the Fc region.

19. The molecule of claim 1, wherein the Fab binds to a tumor antigen and the scFv binds to a T cell antigen; optionally wherein the tumor antigen is BCMA and the T cell antigen is CD3.

20. The molecule of claim 19, wherein the scFv comprises the amino acid sequence of SEQ ID NO: 126 or SEQ ID NO: 128, and wherein the Fab comprises (i) a VH comprising the amino acid sequence of SEQ ID NO: 132, and a VL comprising the amino acid sequence of SEQ ID NO: 129; or (ii) a VH comprising the amino acid sequence of SEQ ID NO: 137, and a VL comprising the amino acid sequence of SEQ ID NO: 135.

21. (canceled)

22. The molecule of claim 20, wherein (a) the VH comprises a Cys at H105; (b) the VL comprises a Cys at L43; (c) the L comprises the amino acid sequence of, SEQ ID NO: 3, SEQ ID NO: 4, SEQ ID NO: 5, SEQ ID NO: 6, or SEQ ID NO: 7; and (d) the scFv is in the VL-L-VH orientation.

23. A polynucleotide encoding the molecule of claim 1 or a fragment thereof.

24. A vector comprising the polynucleotide of claim 23.

25. A host cell comprising the vector of claim 24, optionally wherein the host cell is a prokaryotic cell or an eukaryotic cell.

26. A method of producing a molecule, comprising
a) introducing the polynucleotide of claim 23 into a host cell;
b) culturing the host cell in conditions so that the molecule is produced, and
c) purifying the produced molecule.

27. A method of producing a molecule, comprising:
a) culturing the host cell of claim 25 in conditions so that the molecule is produced, and
b) purifying the produced molecule.

28. A composition comprising the molecule of claim 1, optionally wherein the composition is a pharmaceutical composition further comprising a pharmaceutically acceptable excipient.

29. A method for directing or engaging a cell to a target cell, comprising contacting the target cell with the molecule of claim 1, optionally wherein the Fab binds to a first antigen on the target cell and the scFv binds to a second antigen on the cell; and
wherein:
the cell is an immune cell, optionally wherein the immune cell is a T cell; and
wherein:
the target cell is a tumor cell, and/or the method is for treating a disease or disorder in a subject, optionally wherein a) the disease or disorder is a tumor, optionally wherein the tumor is cancer; and/or b) the subject is a human subject.

30. (canceled)

31. (canceled)

32. A means for producing the molecule of claim 1.

33. A method for eliminating or inhibiting a target cell comprising contacting the target cell with the molecule of claim 1.

34. A method for treating a disease or disorder in a subject comprising administering to the subject the molecule of claim 1.

35. (canceled)

36. (canceled)


SEQUENCE LISTING (150 sequences)

SEQ ID NO: 1 - protein, 4 aa, synthetic construct
  source 1..4: note = Linker
CPPC

SEQ ID NO: 2 - protein, 20 aa, synthetic construct
  source 1..20: note = Linker
GGGGSGGGGSGGGGSGGGGS

SEQ ID NO: 3 - protein, 18 aa, synthetic construct
  source 1..18: note = Linker
GGGSGGSGGCPPCGGSGG

SEQ ID NO: 4 - protein, 16 aa, synthetic construct
  source 1..16: note = Linker
GGGSGGCPPCGGGSGG

SEQ ID NO: 5 - protein, 16 aa, synthetic construct
  source 1..16: note = Linker
GGSGGSGGCPPCGSGG

SEQ ID NO: 6 - protein, 17 aa, synthetic construct
  source 1..17: note = Linker
GGGSGGSGGCPPCGSGG

SEQ ID NO: 7 - protein, 17 aa, synthetic construct
  source 1..17: note = Linker
GGGSGGGSGCPPCGGGG

SEQ ID NO: 8-27 - skipped (20 entries)

SEQ ID NO: 28 - protein, 4 aa, synthetic construct
  source 1..4: note = Linker
CGPC

SEQ ID NO: 29 - protein, 4 aa, synthetic construct
  source 1..4: note = Linker
CPGC

SEQ ID NO: 30 - protein, 4 aa, synthetic construct
  source 1..4: note = Linker
CGGC

SEQ ID NO: 31 - protein, 4 aa, synthetic construct
  source 1..4: note = Linker
CSPG

SEQ ID NO: 32 - protein, 4 aa, synthetic construct
  source 1..4: note = Linker
CPSC

SEQ ID NO: 33 - protein, 4 aa, synthetic construct
  source 1..4: note = Linker
CSSC

SEQ ID NO: 34 - protein, 4 aa, synthetic construct
  source 1..4: note = Linker
CGSC

SEQ ID NO: 35 - protein, 4 aa, synthetic construct
  source 1..4: note = Linker
CSGC

SEQ ID NO: 36 - protein, 5 aa, synthetic construct
  source 1..5: note = Linker
CPPPC

SEQ ID NO: 37 - protein, 5 aa, synthetic construct
  source 1..5: note = Linker
CGPPC

SEQ ID NO: 38 - protein, 5 aa, synthetic construct
  source 1..5: note = Linker
CPGPC

SEQ ID NO: 39 - protein, 5 aa, synthetic construct
  source 1..5: note = Linker
CPPGC

SEQ ID NO: 40 - protein, 5 aa, synthetic construct
  source 1..5: note = Linker
CGGPC

SEQ ID NO: 41 - protein, 5 aa, synthetic construct
  source 1..5: note = Linker
CPGGC

SEQ ID NO: 42 - protein, 5 aa, synthetic construct
  source 1..5: note = Linker
CGGGC

SEQ ID NO: 43 - protein, 5 aa, synthetic construct
  source 1..5: note = Linker
CSPPC

SEQ ID NO: 44 - protein, 5 aa, synthetic construct
  source 1..5: note = Linker
CPSPC

SEQ ID NO: 45 - protein, 5 aa, synthetic construct
  source 1..5: note = Linker
CPPSC

SEQ ID NO: 46 - protein, 5 aa, synthetic construct
  source 1..5: note = Linker
CSSPC

SEQ ID NO: 47 - protein, 5 aa, synthetic construct
  source 1..5: note = Linker
CPSSC

SEQ ID NO: 48 - protein, 5 aa, synthetic construct
  source 1..5: note = Linker
CSSSC

SEQ ID NO: 49 - protein, 5 aa, synthetic construct
  source 1..5: note = Linker
CGSPC

SEQ ID NO: 50 - protein, 5 aa, synthetic construct
  source 1..5: note = Linker
CPGSC

SEQ ID NO: 51 - protein, 5 aa, synthetic construct
  source 1..5: note = Linker
CSGPC

SEQ ID NO: 52 - protein, 5 aa, synthetic construct
  source 1..5: note = Linker
CPSGC

SEQ ID NO: 53-55 - skipped (3 entries)

SEQ ID NO: 56 - protein, 107 aa, synthetic construct [sequence omitted]

SEQ ID NO: 57-59 - skipped (3 entries)

SEQ ID NO: 60 - protein, 119 aa, synthetic construct
  source 1..119: note = Glk1 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSS

SEQ ID NO: 61-62 - skipped (2 entries)

SEQ ID NO: 63 - protein, 15 aa, synthetic construct
  source 1..15: note = Exemplary IgG1 hinge region
EPKSCDKTHTCPPCP

SEQ ID NO: 64 - protein, 22 aa, synthetic construct
  source 1..22: note = Exemplary IgG1 hinge region with lower hinge
EPKSCDKTHTCPPCPAPELLGG

SEQ ID NO: 65 - protein, 12 aa, synthetic construct
  source 1..12: note = Exemplary IgG2 hinge region
ERKCCVECPPCP

SEQ ID NO: 66 - protein, 330 aa, synthetic construct [sequence omitted]

SEQ ID NO: 67 - protein, 326 aa, synthetic construct
  source 1..326: note = wild-type IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 68 - protein, 327 aa, synthetic construct
  source 1..327: note = wild-type IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69-124 - skipped (56 entries)

SEQ ID NO: 125 - protein, 254 aa, synthetic construct
  source 1..254: note = Cris7a VL-VH scFv
EIVLTQSPSAMSASVGDRVTITCSASSSVSYMNWYQQKPGKVPKRLIYDSSKLASGVPSRFSGSGSGTEYTLTISSLQPEDFATYYCQQWSRNPPTFGQGTMLEIKGGGGSGGGGSGGGGSGGGGSEVQLVQSGAEVKKPGESLKISCKGSGYSFTRSTMHWVRQMPGKGLEWMGYINPSSAYTNYNQKFKDQVTISADKSISTAYLQWSSLKASDTAMYYCASPQVHYDYNGFPYWGQGTMVTVSSGHHHHHH

SEQ ID NO: 126 - protein, 250 aa, synthetic construct
  source 1..250: note = Cris7a VL-VH spFv
EIVLTQSPSAMSASVGDRVTITCSASSSVSYMNWYQQKPGCVPKRLIYDSSKLASGVPSRFSGSGSGTEYTLTISSLQPEDFATYYCQQWSRNPPTFGQGTMLEIKGGSGGSGGCPPCGSGGEVQLVQSGAEVKKPGESLKISCKGSGYSFTRSTMHWVRQMPGKGLEWMGYINPSSAYTNYNQKFKDQVTISADKSISTAYLQWSSLKASDTAMYYCASPQVHYDYNGFPYWGCGTMVTVSSGHHHHHH

SEQ ID NO: 127 - protein, 254 aa, synthetic construct
  source 1..254: note = Cris7b VL-VH scFv
EIVLTQSPSAMSASVGDRVTITCSASSSVSYMNWYQQKPGKVPKRLIYDSSKLASGVPSRFSGSGSGTEYTLTISSLQPEDFATYYCQQWSRNPPTFGQGTMLEIKGGGGSGGGGSGGGGSGGGGSQVQLLQSAAEVKKPGESLKISCKGSGYTFTRSTMHWVRQTPGKGLEWMGYINPSSAYTNYNQKFKDQVTISADKSISTAYLQWSSLKASDTAMYYCARPQVHYDYNGFPYWGQGTLVTVSSGHHHHHH

SEQ ID NO: 128 - protein, 250 aa, synthetic construct
  source 1..250: note = Cris7b VL-VH spFv
EIVLTQSPSAMSASVGDRVTITCSASSSVSYMNWYQQKPGCVPKRLIYDSSKLASGVPSRFSGSGSGTEYTLTISSLQPEDFATYYCQQWSRNPPTFGQGTMLEIKGGSGGSGGCPPCGSGGQVQLLQSAAEVKKPGESLKISCKGSGYTFTRSTMHWVRQTPGKGLEWMGYINPSSAYTNYNQKFKDQVTISADKSISTAYLQWSSLKASDTAMYYCARPQVHYDYNGFPYWGCGTLVTVSSGHHHHHH

SEQ ID NO: 129 - protein, 107 aa, synthetic construct
  source 1..107: note = BCMB749_VL
DIVMTQSQKFMSTTVGDRVSITCKASQNVGTAVAWYQQKPGQSPKLLIYSASNRYTGVPDRFTGTGSGTDFTLTIINVQSEDLADYFCQQYGSSPWTFGGGTKLEIK

SEQ ID NO: 130 - protein, 107 aa, synthetic construct [sequence omitted]

SEQ ID NO: 131 - protein, 214 aa, synthetic construct
  source 1..214: note = BCMB749 LC
DIVMTQSQKFMSTTVGDRVSITCKASQNVGTAVAWYQQKPGQSPKLLIYSASNRYTGVPDRFTGTGSGTDFTLTIINVQSEDLADYFCQQYGSSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 118 aa, synthetic construct
  source 1..118: note = BCMB749_VH
QVQLQQSGAELVKPGASVKLSCKASGYTFTNNVMHWVRQKPGQGLEWIGYILPYNDGTKYNEKFKGKATLTSDKSSSTAYMELSSLTSEDSAVYYCARYDYDGYFDYWGQGTTLTVSS

SEQ ID NO: 133 - protein, 330 aa, synthetic construct
  source 1..330: note = Human_HC_ConstantDomains 1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 134 - protein, 448 aa, synthetic construct
  source 1..448: note = BCMB749 HC1
QVQLQQSGAELVKPGASVKLSCKASGYTFTNNVMHWVRQKPGQGLEWIGYILPYNDGTKYNEKFKGKATLTSDKSSSTAYMELSSLTSEDSAVYYCARYDYDGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 135 - protein, 107 aa, synthetic construct
  source 1..107: note = BCMB749h_VL
DIQMTQSPSSVSASVGDRVTITCKASQNVGTAVAWYQQKPGQSPKLLIYSASNRYTGVPSRFSGSGSGTDFTLTISSLQPEDFATYFCQQYGSSPWTFGGGTKVEIK

SEQ ID NO: 136 - protein, 214 aa, synthetic construct
  source 1..214: note = BCMB749h LC
DIQMTQSPSSVSASVGDRVTITCKASQNVGTAVAWYQQKPGQSPKLLIYSASNRYTGVPSRFSGSGSGTDFTLTISSLQPEDFATYFCQQYGSSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 118 aa, synthetic construct
  source 1..118: note = BCMB749h_VH
QVQLVQSGAEVKKPGASVKVSCKASGYTFTNNVMHWVRQAPGQGLEWMGYILPYNDGTKYNQKFQGRVTLTSDKSASTAYMELSSLRSEDTAVYYCARYDYDGYFDYWGQGTTVTVSS

SEQ ID NO: 138 - protein, 448 aa, synthetic construct
  source 1..448: note = BCMB749h HC1
QVQLVQSGAEVKKPGASVKVSCKASGYTFTNNVMHWVRQAPGQGLEWMGYILPYNDGTKYNQKFQGRVTLTSDKSASTAYMELSSLRSEDTAVYYCARYDYDGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 139 - protein, 232 aa, synthetic construct
  source 1..232: note = Human_HC_ConstantDomains 2
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 479 aa, synthetic construct
  source 1..479: note = Cris7b VL-VH scFv HC2
EIVLTQSPSAMSASVGDRVTITCSASSSVSYMNWYQQKPGKVPKRLIYDSSKLASGVPSRFSGSGSGTEYTLTISSLQPEDFATYYCQQWSRNPPTFGQGTMLEIKGGSEGKSSGSGSESKSTGGSQVQLLQSAAEVKKPGESLKISCKGSGYTFTRSTMHWVRQTPGKGLEWMGYINPSSAYTNYNQKFKDQVTISADKSISTAYLQWSSLKASDTAMYYCARPQVHYDYNGFPYWGCGTLVTVSSEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 477 aa, synthetic construct
  source 1..477: note = Cris7b VL-VH spFv HC2
EIVLTQSPSAMSASVGDRVTITCSASSSVSYMNWYQQKPGCVPKRLIYDSSKLASGVPSRFSGSGSGTEYTLTISSLQPEDFATYYCQQWSRNPPTFGQGTMLEIKGGGSGGSGGCPPCGGSGGQVQLLQSAAEVKKPGESLKISCKGSGYTFTRSTMHWVRQTPGKGLEWMGYINPSSAYTNYNQKFKDQVTISADKSISTAYLQWSSLKASDTAMYYCARPQVHYDYNGFPYWGCGTLVTVSSEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 486 aa, synthetic construct
  source 1..486: note = CD3B219a99v scFv HC2
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGSEGKSSGSGSESKSTGGSEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCVRHGNFGNSYVSWFAYWGQGTLVTVSSEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 484 aa, synthetic construct
  source 1..484: note = CD3B219a99v spFv HC2
QTVVTQEPSLTVSPGGTVTLTCRSSTGAVTTSNYANWVQQKPGCAPRGLIGGTNKRAPGTPARFSGSLLGGKAALTLSGVQPEDEAEYYCALWYSNLWVFGGGTKLTVLGGGSGGSGGCPPCGGSGGEVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCVRHGNFGNSYVSWFAYWGCGTLVTVSSEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 6 aa, synthetic construct
  source 1..6: note = Linker
  VARIANT 6: note = X is any amino acid (most often G or S)
  VARIANT 1: note = can be repeated n times, where n is an integer from 7 to 9
  VARIANT 6: note = can be repeated m times, where m is an integer from 4 to 5
  VARIANT 1: note = X is any amino acid (most often G or S)
XCPPCX

SEQ ID NO: 145 - protein, 108 aa, synthetic construct
  source 1..108: note = Glk2 VL
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIK

SEQ ID NO: 146 - protein, 118 aa, synthetic construct
  source 1..118: note = Glk2 VH
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSS

SEQ ID NO: 147 - protein, 110 aa, synthetic construct
  source 1..110: note = CAT2200 VL
NFMLTQPHSVSESPGKTVTISCTRSSGSLANYYVQWYQQRPGSSPTIVIFANNQRPSGVPDRFSGSIDSSSNSASLTISGLKTEDEADYYCQTYDPYSVVFGGGTKLTVL

SEQ ID NO: 148 - protein, 118 aa, synthetic construct
  source 1..118: note = CAT2200a VH
EVQLLESGGGLVQPGGSLRLSCAASGFGFSSYAMSWVRQAPGKGLEWVSAISGSGGSTYYADSVKGRFTISRDNSKNTLYLQMNSLRAEDTAVYYCARDLIHGVTRNWGQGTLVTVSS

SEQ ID NO: 149 - protein, 105 aa, synthetic construct
  source 1..105: note = huKV1-12*01
DIQMTQSPSSVSASVGDRVTITCRASQGISSWLAWYQQKPGKAPKLLIYAASSLQSGVPSRFSGSGSGTDFTLTISSLQPEDFATYYCQQANSFPFGGGTKVEIK

SEQ ID NO: 150 - protein, 109 aa, synthetic construct
  source 1..109: note = huHV1-3*01
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARWGQGTTVTVSS